(19) United States
(12) Patent Application Publication
(10) Pub. No.: US 2013/0287700 A1
(43) Pub. Date: Oct. 31, 2013

(54) COMPOUNDS FOR THE DIAGNOSIS OF NEURODEGENERATIVE DISORDERS ON THE OLFACTORY EPITHELIUM

(71) Applicants: Boris Schmidt, Darmstadt (DE); Daniel Kieser, Wixhausen (DE); Alexander Boländer, Griesheim (DE); Jochen Herms, Neuried (DE); Roland Heyny-Von Haussen, Darmstadt (DE); Jiamin Gu, Darmstadt (DE)

(72) Inventors: Boris Schmidt, Darmstadt (DE); Daniel Kieser, Wixhausen (DE); Alexander Boländer, Griesheim (DE); Jochen Herms, Neuried (DE); Roland Heyny-Von Haussen, Darmstadt (DE); Jiamin Gu, Darmstadt (DE)

(73) Assignees: KLINIKUM DARMSTADT GMBH, Darmstadt (DE); TECHNISCHE UNIVERSITAT DARMSTADT, Darmstadt (DE); LUDWIG-MAXIMILIANS-UNIVERSITAT MUNCHEN, Munchen (DE)

(21) Appl. No.: 13/825,186

(22) PCT Filed: Sep. 20, 2011
(86) PCT No.: PCT/DE2011/001780

§ 371 Date: Jul. 18, 2013

(30) Foreign Application Priority Data
Sep. 20, 2010 (DE) 102010045797.3

(51) Int. Cl. A61K 49/00; G01N 21/64

(52) U.S. Cl. CPC A61K 49/0021; G01N 21/6428
USPC 424/9.6; 436/501; 544/242; 544/316; 544/333; 544/336; 544/405; 548/159; 546/270.1

(57) ABSTRACT

Subject of the present invention are compounds with high affinity for the Aβ protein, α-synuclein or for Tau-PHF aggregates, which are suitable as preferably fluorescent probes for the in vivo diagnosis of neurodegenerative disorders like e.g. Alzheimer's disease and Parkinson's disease. The compounds are characterized by suitable physicochemical properties (excitation wavelength, emission wavelength, Stokes shift, extinction) as well as a high affinity and selectivity for the target proteins.

The present invention describes compounds with affinity for the Aβ protein, α-synuclein or for Tau-PHF aggregates which are suitable as preferably fluorescent probes for the in vivo diagnosis of neurodegenerative disorders like e.g. Alzheimer's disease and Parkinson's disease. The compounds are distinguished by suitable physicochemical properties (excitation wavelength, emission wavelength, Stokes shift, extinction) as well as a high affinity and selectivity for the target proteins.

Patients in therapy substantially benefit from an early diagnosis of neurodegenerative disorders like Alzheimer's disease or Parkinson's disease. A reliable diagnosis of Alzheimer's disease is however, in particular in early stages of the disease, at present only possible with post-mortem microscopic investigations.

Current methods applied in the living Alzheimer's patient are heterogeneous and based on third party anamneses, imaging techniques, cognitive tests and the exclusion of other neurodegenerative disorders. The diagnosis in the living patient, in particular in early stages of the disease, can often not clearly be distinguished from other dementias. At a time point when the first symptoms become apparent in the short-term memory, already substantial pathological changes have occurred in the brain. These changes can partly be detected with so-called non-invasive investigation methods like e.g. computed tomography (CT scan), magnetic resonance tomography (MRT) and positron emission tomography (PET).

WO2009155017 discloses radio-pharmaceutical compositions having high affinity for amyloid plaques which can be detected by positron emission tomography.

WO2007136996 discloses cyanine dyes which are suitable for the labeling of biomolecules, e.g. for the in vivo diagnosis of tumor diseases.

US20020133019 discloses thioflavin derivatives for the ante mortem in vivo diagnosis of among others also Alzheimer's disease. Labeled thioflavins bind to amyloid plaques and are detected via gamma-imaging, MRT or NMR spectroscopy.

None of these methods and dyes is able to detect early disease stages, and in the case of positron emission tomography, a highly expensive and not widespread infrastructure is required.

The removal of cerebrospinal fluid (liquor cerebrospinalis) for the detection of increased Aβ-values requires spinal cord puncture. It thus represents an invasive investigation method which is associated with a considerably high rate of complications and thus unsuitable to be used for predictive diagnostics.

The objective of the present invention is therefore to provide suitable diagnostic probes for the detection of neurodegenerative disorders which can be performed at the olfactory epithelium and/or bulbus olfactorius using an optical detection method.

The present invention solves this objective by use of specific compounds for the diagnosis of neurodegenerative disorders. In specific embodiments of the invention these compounds have at least three of the following properties a)-f):
a) a >10fold extinction increase upon binding to the Aβ protein, α-synuclein or to Tau-PHF aggregates as compared to the free compound,
b) a Stokes shift of >20 nm,
c) an extinction coefficient of ε>10 000 L·mol⁻¹·cm⁻¹,
d) EC50<300 nM
e) a log P value between 1 and 2.8,
f) a topological polar surface area (TPSA)<70 Å².

Preferred are compounds, also called probes, whose conformation in an excited state is stabilized by binding at the target protein. This stabilization can occur by hydrogen bonds or van der Waals interactions of the aryl units with the target protein. This leads to a stabilization of the non-planar, excited state for preferred probes. Such a twist can be induced by 1,3 allyl tension or by 1,4 butadien tension of alkylated vinyl aromatics. Hence, compounds or probes are preferred with a styryl unit, i.e. a vinyl group at a benzene ring or generally a C═C double bond or C═O double bond or C═N double bond or N═N double bond at an aryl compound or heteroaryl compound. Aryl compound or heteroaryl compound being bond to a C═C, C═O, C═N or N═N group, preferably are benzene, naphthalene, toluene, xylene, pyridine, pyrazine, pyrimidine, pyridazine or 1,2,5 triazine. In particular preferred compounds or probes are aromatic molecules which have a π electron system expanded over at least two aromatic rings or aromatic ring systems. Thus, preferred compounds or probes have a system of (4n+2) delocalized electrons, wherein n=1, 2, 3, 4 or 5 is.

In particular compounds of the following classes of substances have three or more of the aforementioned properties, so that the present invention relates to the provision of arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones or diaryl ureas which have affinity for the Aβ protein, α-synuclein or Tau-PHF aggregates and are thus suitable for the diagnosis and treatment of neurodegenerative disorders.

Compounds of the substance class of arylaminothiazoles preferably have the following general structure:

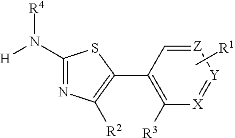

wherein

X, Y, Z are, independently of one another, carbon or nitrogen and

R¹, R², R³, R⁴ are, independently of one another, C₁-C₆-alkyl, C₂-C₆-alkenyl, C₂-C₆-alkynyl, C₂-C₆-alkenynyl, C₁-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₂-C₆-haloalkenynyl, C₁-C₁₀-halocycloalkyl, —H, —OH, —OCH₃, —OC₂H₅,

—OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH— as well as salts, enantiomers, enantiomeric mixtures, diastereomers, diastereomeric mixtures, tautomers, hydrates, solvates and racemates of the aforementioned compounds.

Preferred are divinylaryls, more preferred are divinylnitrogenheteroaryls and particularly preferred are divinylpyrimidines, divinylpyridines, divinylpyrazines, divinylpyridazines and divinyltriazoines.

Compounds of the substance class of 4,6-divinylpyrimidines comprise compounds, which preferably have the following general structure:

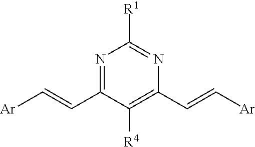

wherein

Ar represents one of the following cyclic, heterocyclic, aromatic or heteroaromatic groups:

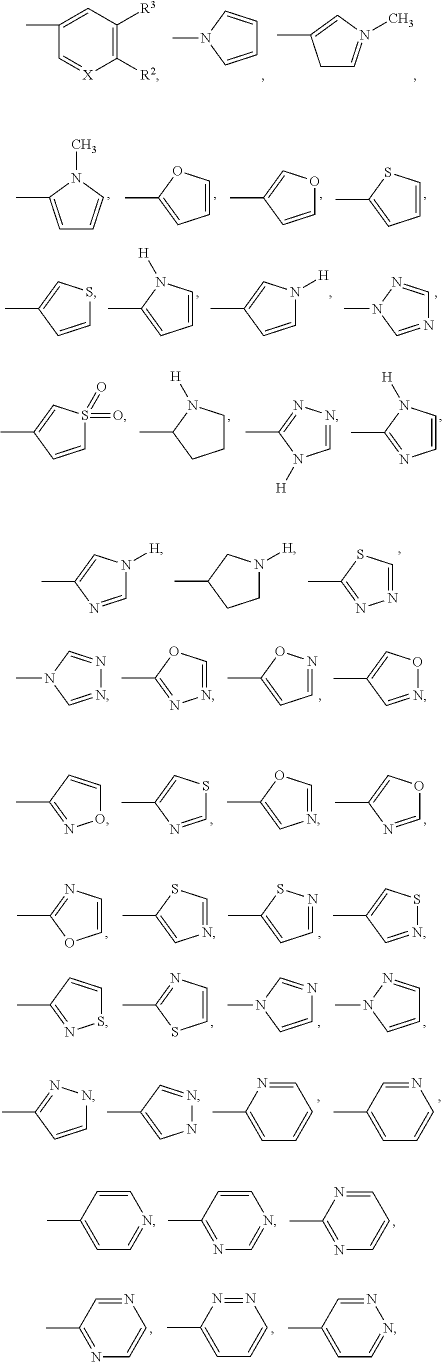

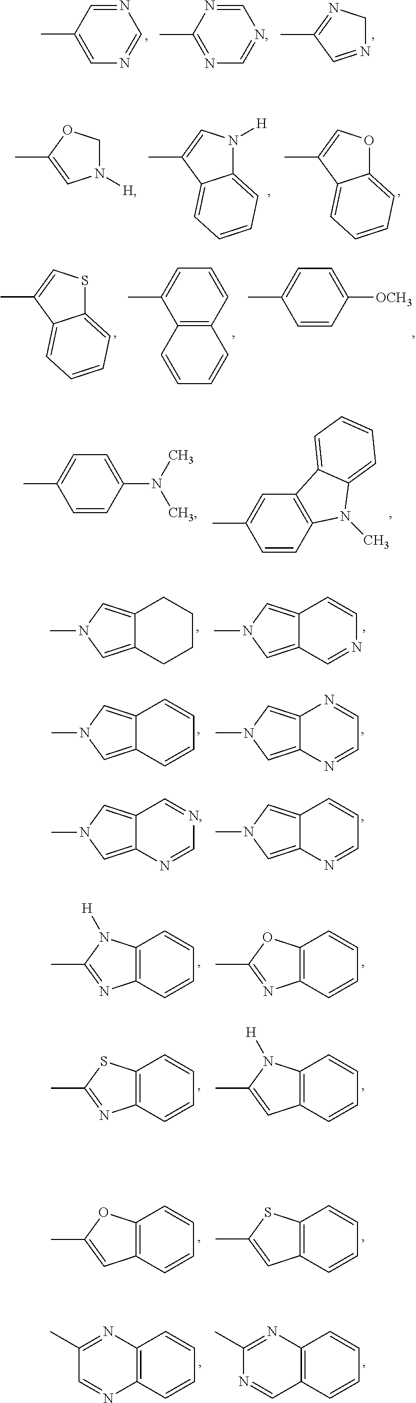

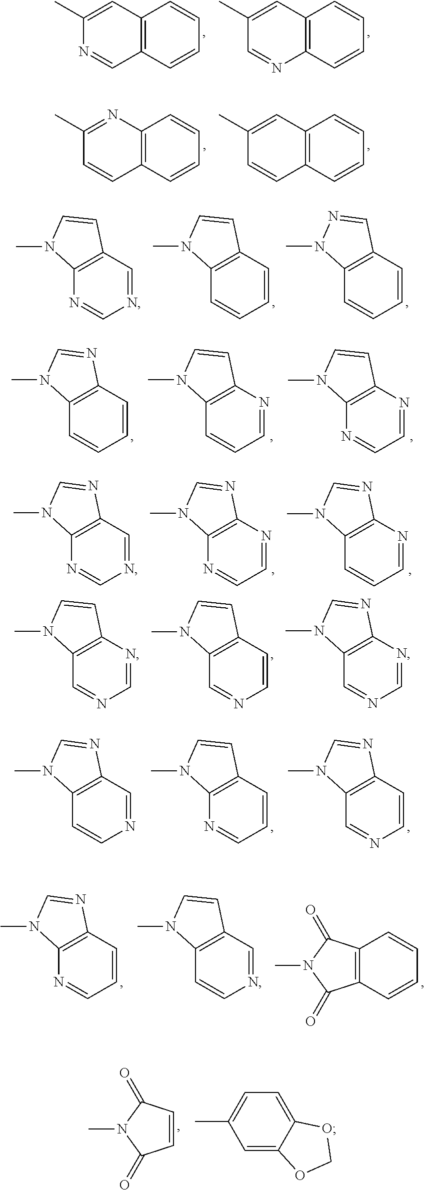

wherein

X is carbon or nitrogen and

R¹, R², R³ and R⁴ are, independently of one another, C₁-C₆-alkyl, C₂-C₆-alkenyl, C₂-C₆-alkynyl, C₄-C₆-alkeninyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-Haloalkenynyl, C₃-C₁₀-halocycloalkyl, —H, —OH, —OCH₃, —OC₂H₅,

—OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph oder —CH═CH-Ph as well as salts, enantiomers, enantiomeric mixtures, diastereomers, diastereomeric mixtures, tautomers, hydrates, solvates and racemates of the aforementioned compounds.

Compounds of the substance class of 2,5-divinylpyrazines comprise compounds which preferably have the following general structure:

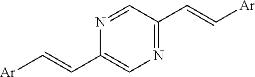

Wherein

Ar represents one of the following cyclic, heterocyclic, aromatic or heteroaromatic groups:

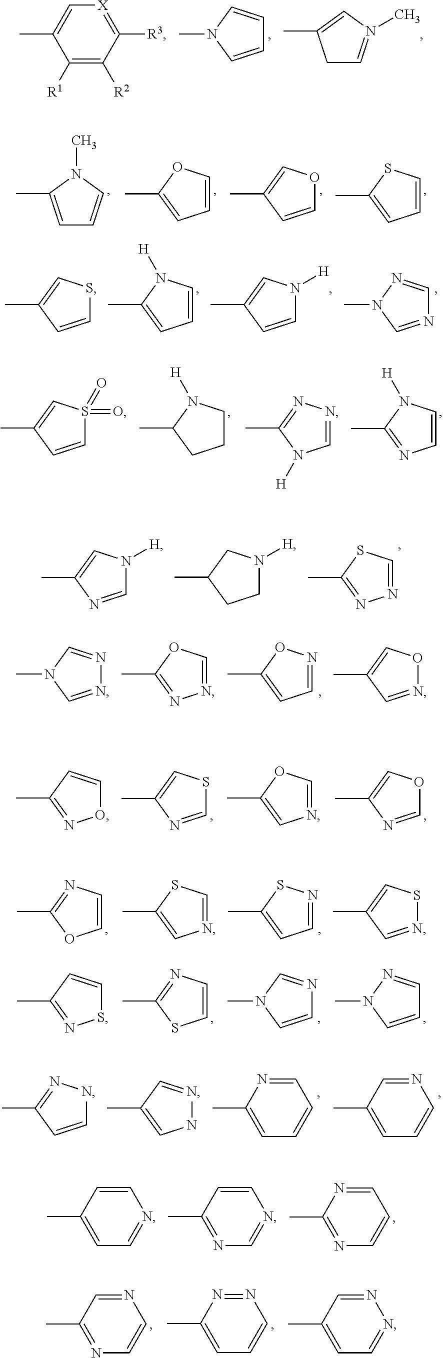

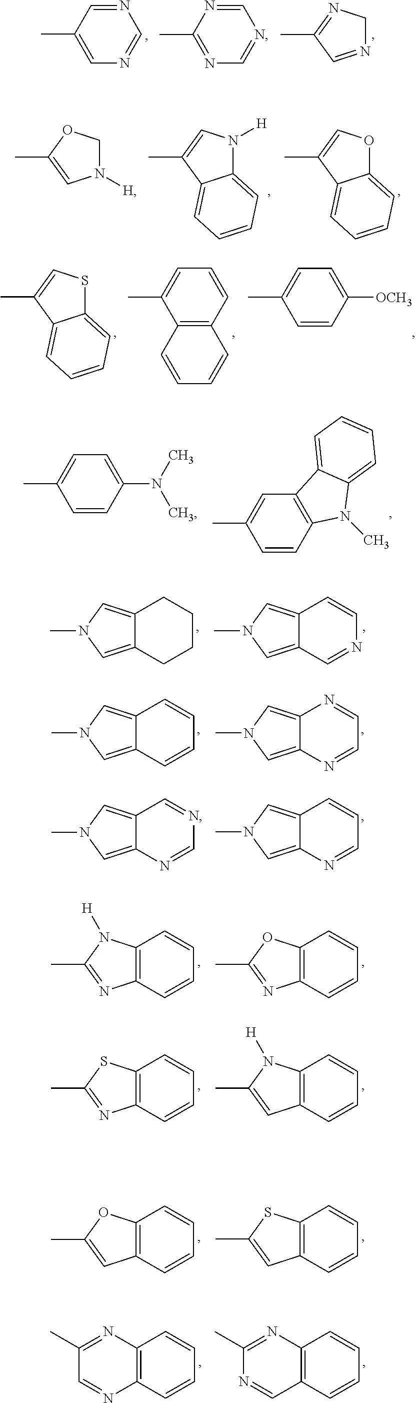

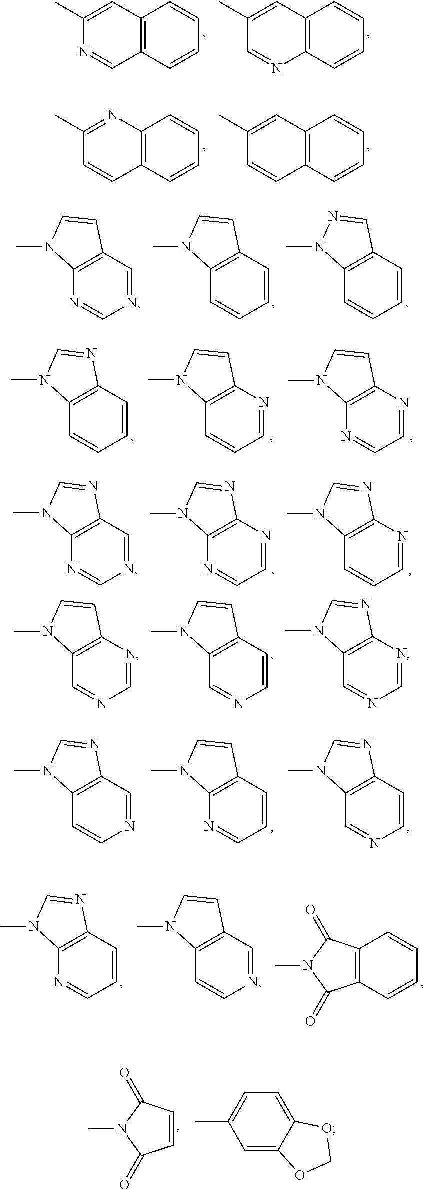

wherein

X is carbon or nitrogen and

R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅,

—OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph as well as salts, enantiomers, enantiomeric mixtures, diastereomers, diastereomeric mixtures, tautomers, hydrates, solvates and racemates of the abovementioned compounds.

Compounds of the substance class of [4-(1,3-benzothiazole-2-yl)phenyl]hydrazones comprise compounds which preferably have the following structure:

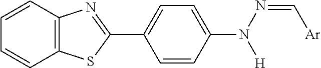

wherein

Ar represents one of the following cyclic, heterocyclic, aromatic or heteroaromatic groups:

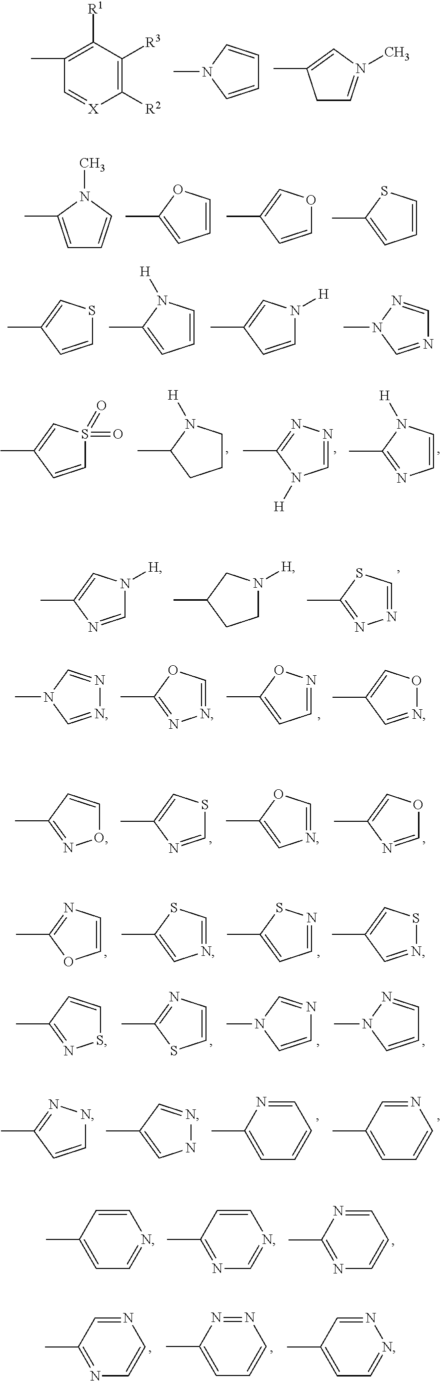

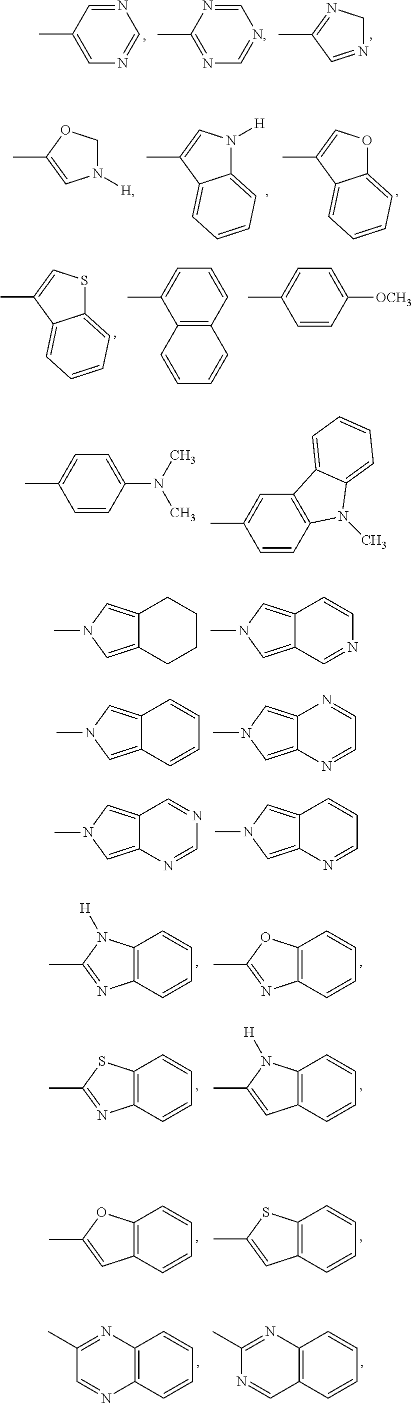

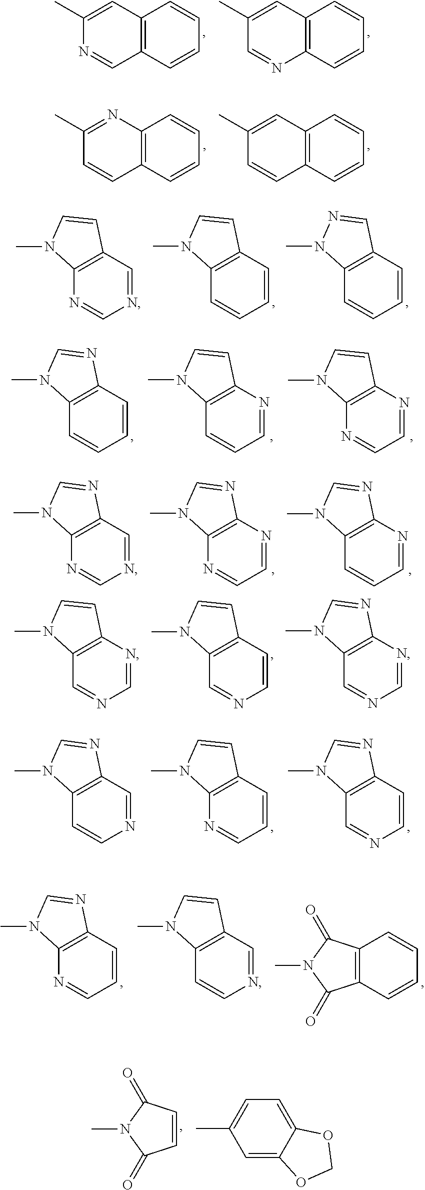

wherein

X is carbon or nitrogen, and

R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅,

—OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph as well as salts, enantiomers, enantiomeric mixtures, diastereomers, diastereomeric mixtures, tautomers, hydrates, solvates and racemates of the aforementioned compounds.

Compounds of the substance class of 3,6-divinylpyridazines comprise compounds which preferably have the following general structure:

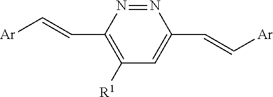

wherein

Ar represents one of the following cyclic, heterocyclic, aromatic or heteroaromatic groups:

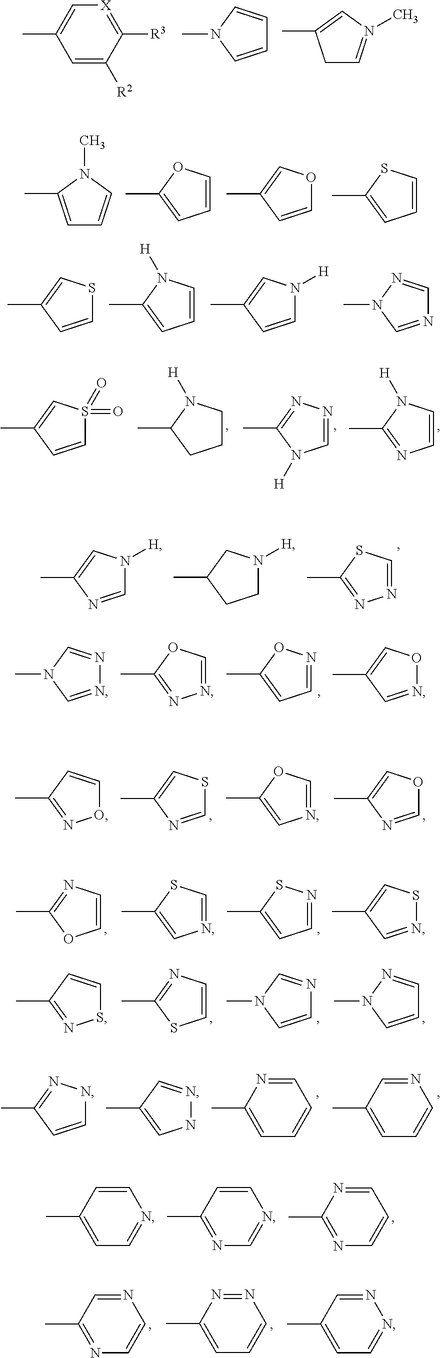

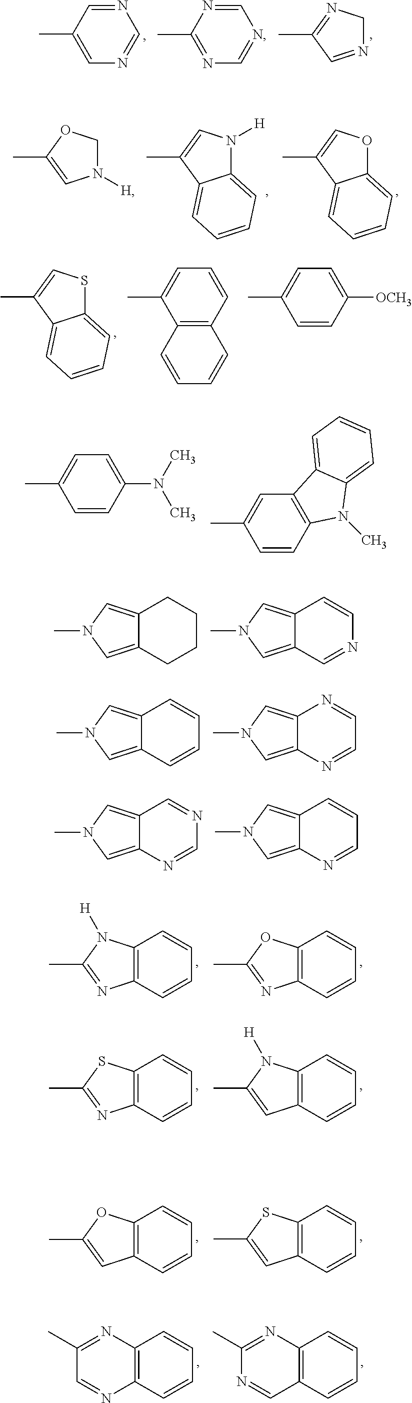

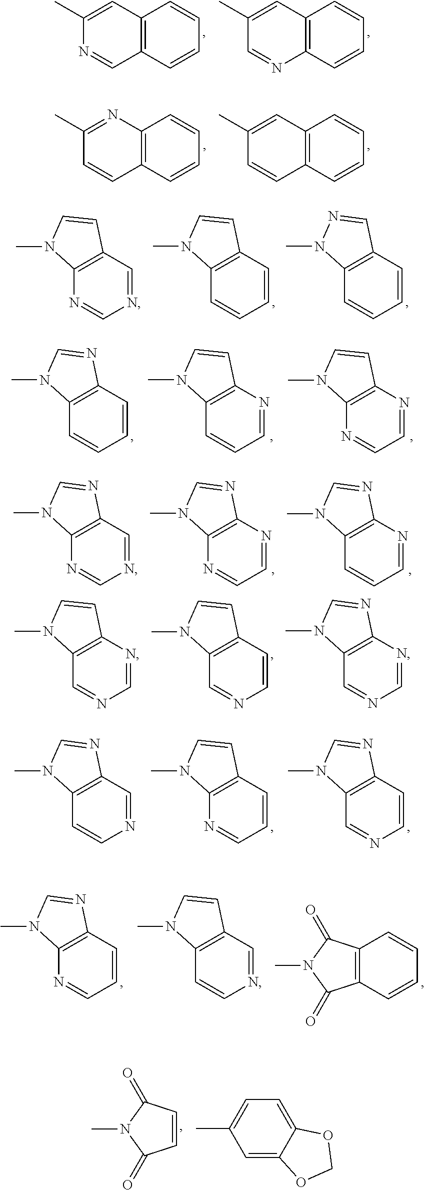

wherein X is carbon or nitrogen and

R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₃-C₆-haloalkenynyl, C₃-C₁₀-halocycloalkyl, —H, —OH, —OCH₃, —OC₂H₅,

—OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph as well as salts, enantiomers, enantiomeric mixtures, diastereomers, diastereomeric mixtures, tautomers, hydrates, solvates and racemates of the aforementioned compounds.

Compounds of the substance class of diaryl ureas comprise compounds which preferably have the following general structure:

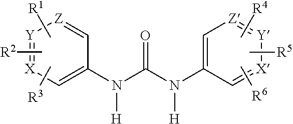

wherein

X, X′, Y, Y′, Z, Z′ are, independently of one another, carbon or nitrogen, and

R¹, R², R³, R⁴, R⁵, R⁶ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph as well as salts, enantiomers, enantiomeric mixtures, diastereomers, diastereomeric mixtures, tautomers, hydrates, solvates and racemates of the aforementioned compounds.

Compounds of the substance class of 2H-indol-2-yliden-1-propen-1-yl-indolium cations, benzothiazolyliden-1-propenyl-benzothiazolium cations and benzoxazolyliden-1-propenyl-benzoxazolium cationen comprise compounds which preferably have the following general structure:

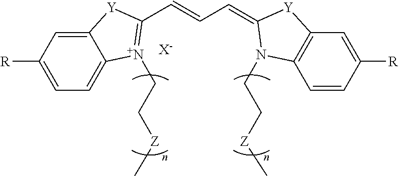

wherein

R represents hydrogen, —F, —Cl, —Br, —I, —NO₂, alkoxy;

X represents —Cl, —Br, —I, —OTs, —OMs;

Y represents O, S, CR¹R²;

wherein R¹ and R² independently of one another represent —CH₃ or —C₂H₅;

Z represents O or CH₂; and

n represents 0, 1, 2 or 3.

As used herein, “C₁-C₆-alkyl” means —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, and —CH(CH₃)—C(CH₃)₃.

Preferred are —CH₃, —C₂H₅, —C₃H₇, —CH(CH₃)₂, —C₄H₉, —CH₂—CH(CH₃)₂, —CH(CH₃)—C₂H₅, —C(CH₃)₃ and —C₅H₁₁. Particularly preferred are —CH₃, —C₂H₅, —C₃H₇ and —CH(CH₃)₂.

As used herein, “C₂-C₆-alkenyl” means —CH═CH₂, —CH₂—CH═CH₂, —C(CH₃)═CH₂, —CH═CH—CH₃, —C₂H₄—CH═CH₂, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃)—CH═CH—CH₃, —CH═CH—CH(CH₃)₂, —CH═C(CH₃)—C₂H₅, —C(CH₃)═CH—C₂H₅, —C(CH₃)═C(CH₃)₂, —C(CH₃)₂—CH═CH₂, —CH(CH₃)—C(CH₃)═CH₂, —C(CH₃)═CH—CH═CH₂, —CH═C(CH₃)—CH═CH₂, —CH═CH—C(CH₃)═CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)—CH═CH₂, —CH₂—CH(CH₃)—CH₂—CH═CH₂, —CH(CH₃)—C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)═CH—C₃H₇, —CH₂—CH(CH₃)—C(CH₃)═CH₂, —CH(CH₃)—CH₂—C(CH₃)═CH₂, —CH(CH₃)—CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —CH═CH—C(CH₃)₃, —C(CH₃)₂—C(CH₃)═CH₂, —CH(C₂H₅)—C(CH₃)═CH₂, —C(CH₃)(C₂H₅)—CH═CH₂, —CH(CH₃)—C(C₂H₅)═CH₂, —CH₂—C(C₃H₇)═CH₂, —CH₂—C(C₂H₅)═CH—CH₃, —CH(C₂H₅)—CH═CH—CH₃, —C(C₄H₉)═CH₂, —C(C₃H₇)═CH—CH₃, —C(C₂H₅)═CH—C₂H₅, —C(C₂H₅)═C(CH₃)₂, —C[C(CH₃)₃]═CH₂, —C[CH(CH₃)(C₂H₅)]═CH₂, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH₂—CH═CH—CH═CH—CH₃, —CH═CH—CH₂—CH═CH—CH₃, —CH═CH—CH═CH—C₂H₅, —CH₂—CH═CH—C(CH₃)═CH₂, —CH₂—CH═C(CH₃)—CH═CH₂, —CH₂—C(CH₃)═CH—CH═CH₂, —CH(CH₃)—CH═CH—CH═CH₂, —CH═CH—CH₂—C(CH₃)═CH₂, —CH═CH—CH(CH₃)—CH═CH₂, —CH═C(CH₃)—CH₂—CH═CH₂, —C(CH₃)═CH—CH₂—CH═CH₂, —CH═CH—CH═C(CH₃)₂, —CH═CH—C(CH₃)═CH—CH₃, —CH═C(CH₃)—CH═CH—CH₃, —C(CH₃)═CH—CH═CH—CH₃, —CH═C(CH₃)—C(CH₃)═CH₂, —C(CH₃)═CH—C(CH₃)═CH₂, —C(CH₃)═C(CH₃)—CH═CH₂ or —CH═CH—CH═CH—CH═CH₂.

Preferred are —CH═CH₂, —CH₂—CH═CH₂, —C(CH₃)═CH₂, —CH═CH—CH₃, —C₂H₄—CH═CH₂, —CH₂—CH═CH—CH₃. Particularly preferred are —CH═CH₂, —CH₂—CH═CH₂ and —CH═CH—CH₃.

As used herein, “C₂-C₆-alkynyl” means —C≡CH, —C≡C—CH₃, —CH₂—C≡CH, —C₂H₄—C≡CH, —CH₂—C≡C—CH₃, —C≡C—C₂H₅, —C₃H₆—C≡CH, —CH₂H₄—C≡C—CH₃, —CH₂—C≡C—C₂H₅, —C≡C—C₃H₇, —CH(CH₃)—C≡CH, —CH₂—CH(CH₃)—C≡CH, —CH(CH₃)—CH₂—C≡CH, —CH(CH₃)—C≡C—CH₃, —C₄H₈—C≡CH, —C₃H₆—C≡C—CH₃, —C₂H₄—C≡C—C₂H₅, —CH₂—C≡C—C₃H₇, —C≡C—C₄H₉, —C₂H₄—CH(CH₃)—C≡CH, —CH₂—CH(CH₃)—CH₂—C≡CH, —CH(CH₃)—C₂H₄—C≡CH, —CH₂—CH(CH₃)—C≡C—CH₃, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —C═C—CH₂—CH(CH₃)₂, —C≡C—C(CH₃)₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —C(CH₃)(C₂H₅)—C≡CH, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —CH₂—C≡C—C≡C—CH₃, —C≡C—CH₂—C≡C—CH₃, —C≡C—C≡C—C₂H₅, —C≡C—CH(CH₃)—C≡CH, —CH(CH₃)—C≡C—C≡CH, —CH(C≡CH)—CH₂—C≡CH, —C(C≡CH)₂—CH₃, —CH₂—CH(C≡CH)₂ or —CH(C≡CH)—C≡C—CH₃.

Preferred are —C≡CH and —C≡C—CH₃.

As used herein, “C₄-C₆-alkenynyl” means —C≡C—CH═CH₂, —CH═CH—C≡CH, —CH₂—C≡C—CH═CH₂, —CH₂—CH═CH—C≡CH, —C≡C—CH═CH—CH₃, —CH═CH—C≡C—CH₃, —C≡C—CH₂—CH═CH₂, —CH═CH—CH₂—C≡CH, —C≡C—CH₂—C≡CH, —C(CH₃)═CH—C≡CH, —CH═C(CH₃)—C≡CH, —C≡C—C(CH₃)═CH₂, or —C≡C—C≡C—C≡CH.

As used herein, “C₃-C₁₀-cycloalkyl” means

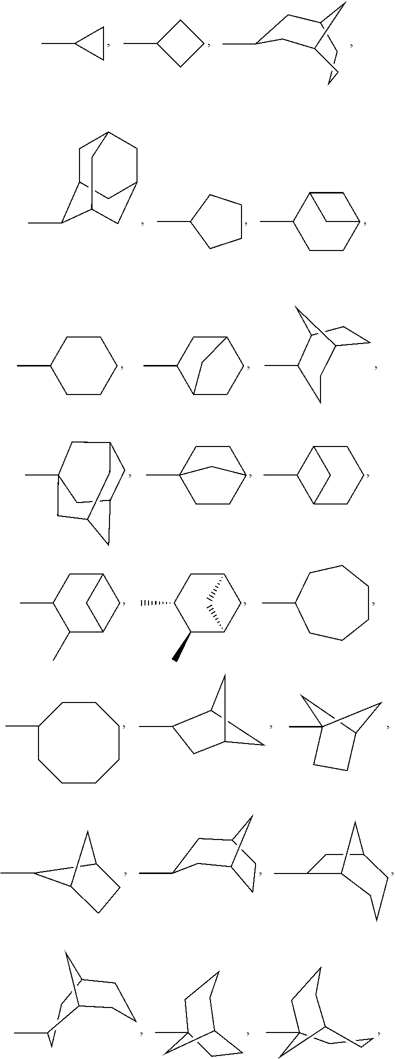

As used herein, “thioalkyl” means —S—C₁-C₆-alkyl, wherein C₁-C₆-alkyl has the above mentioned meaning. Preferred are the following groups —S—CH₃, —S—C₂H₅, —S—C₃H₇, —S—CH(CH₃)₂, —S—C₄H₉, —S—CH₂—CH(CH₃)₂, —S—CH(CH₃)—C₂H₅, —S—C(CH₃)₃ and —S—C₅H₁₁. Particularly preferred are —S—CH₃, —S—C₂H₅, —S—C₃H₇, —S—CH(CH₃)₂ and —S—C(CH₃)₃.

As used herein, C₁-C₆-haloalkyl means a C₁-C₆-alkyl group containing at least one halogen atom chosen from fluorine, chlorine, bromine, iodine. Preferred are the groups —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br and —CH₂—CH₂I.

Correspondingly, C₂-C₆-haloalkenyl means a C₂-C₆-alkenyl group containing at least one halogen atom chosen from fluorine, chlorine, bromine, iodine.

C₂-C₆-haloalkynyl means a C₂-C₆-alkynyl group containing at least one halogen atom chosen from fluorine, chlorine, bromine, iodine. C₄-C₆-haloalkenynyl means a C₄-C₆-alkenynyl containing at least one halogen atom chosen from fluorine, chlorine, bromine, iodine, and C₃-C₁₀-halocycloalkyl means a C₁-C₁₀-cycloalkyl group containing at least one halogen atom chosen from fluorine, chlorine, bromine, iodine.

As used herein, “alkyloxy” or “alkoxy” means —O—C₁-C₆-alkyl, wherein C₁-C₆-alkyl has the abovementioned meaning. Preferred are the following C₁-C₆ alkoxy groups —O—CH₃, —O—C₂H₅, —O—C₃H₇, —O—CH(CH₃)₂, —O—C₄H₉, —O—CH₂—CH(CH₃)₂, —O—CH(CH₃)—C₂H₅, —O—C(CH₃)₃ and —O—C₅H₁₁. Particularly preferred are —O—CH₃, —O—C₂H₅, —O—C₃H₇, —O—CH(CH₃)₂ and —O—C(CH₃)₃.

As used herein, “C₁-C₆-alkanoyl” or “acyl” means a C₆-C₁₆-aryl- or C₁-C₆-alkyl group which is bound via a carbonyl function (—C(═O)—) as specified in the following: —CO—C₁-C₆-alkyl, wherein C₁-C₆-alkyl has the abovementioned meaning, or —CO—C₆-C₁₆-aryl, and “aryl” represents phenyl, substituted phenyl, naphthyl, substituted naphthyl, heteroaryl or substituted heteroaryl. Preferred are —CO—CH₃, —CO—C₂H₅, —CO—C₃H₇, —CO—CH(CH₃)₂, —CO—C₄H₉, —CO—CH₂—CH(CH₃)₂, —CO—CH(CH₃)—C₂H₅, —CO—C(CH₃)₃ and —CO—C₅H₁₁. Particularly preferred are —CO—CH₃, —CO—C₂H₅, —CO—C₃H₇, —CO—CH(CH₃)₂ and —CO—C(CH₃)₃.

Preferred substituents are the following:

—H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —P(O)(OH)₂, —P(O)(OCH₃)₂, —P(O)(OC₂H₅)₂, —P(O)(OCH(CH₃)₂)₂, —C(OH)[P(O)(OH)₂]₂, —Si(CH₃)₂(C(CH₃)₃), —Si(C₂H₅)₃, —Si(CH₃)₃, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₂NH₂, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —NH—C(═NH)—NH₂, —NH—C(═NH)—NHCH₃, —NH—C(═NH)—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —NH—C(═NH)—N[CH(CH₃)₂]₂, —NH—C(═NH)—N[C(CH₃)₃]₂, —O—CO—NH₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₇H₁₅, —C₈H₁₇, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃)—CH═CH—CH₃, —CH═CH—CH(CH₃)₂, —CH═C(CH₃)—C₂H₅, —C(CH₃)═CH—C₂H₅, —C(CH₃)═C(CH₃)₂, —C(CH₃)₂—CH═CH₂, —CH(CH₃)—C(CH₃)═CH₂, —C(CH₃)═CH—CH═CH₂, —CH═C(CH₃)—CH═CH₂, —CH═CH—C(CH₃)═CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)—CH═CH₂, —CH₂—CH(CH₃)—CH₂—CH═CH₂, —CH(CH₃)—C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)═CH—C₃H₇, —CH₂—CH(CH₃)—C(CH₃)═CH₂, —CH(CH₃)—CH₂—C(CH₃)═CH₂, —CH(CH₃)—CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —CH═CH—C(CH₃)₃, —C(CH₃)₂—C(CH₃)═CH₂, —CH(C₂H₅)—C(CH₃)═CH₂, —C(CH₃)(C₂H₅)—CH═CH₂, —CH(CH₃)—C(C₂H₅)═CH₂, —CH₂—C(C₃H₇)═CH₂, —CH₂—C(C₂H₅)═CH—CH₃, —CH(C₂H₅)—CH═CH—CH₃, —C(C₄H₉)═CH₂, —C(C₃H₇)═CH—CH₃, —C(C₂H₅)═CH—C₂H₅, —C(C₂H₅)═C(CH₃)₂, —C[C(CH₃)₃]═CH₂, —C[CH(CH₃)(C₂H₅)]═CH₂, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH₂—CH═CH—CH═CH—CH₃, —CH═CH—CH₂—CH═CH—CH₃, —CH═CH—CH═CH—C₂H₅, —CH₂—CH═CH—C(CH₃)═CH₂, —CH₂—CH═C(CH₃)—CH═CH₂, —CH₂—C(CH₃)═CH—CH═CH₂, —CH(CH₃)—CH═CH—CH═CH₂, —CH═CH—CH₂—C(CH₃)═CH₂, —CH═CH—CH(CH₃)—CH═CH₂, —CH═C(CH₃)—CH₂—CH═CH₂, —C(CH₃)═CH—CH₂—CH═CH₂, —CH═CH—CH═C(CH₃)₂, —CH═CH—C(CH₃)═CH—CH₃, —CH═C(CH₃)—CH═CH—CH₃, —C(CH₃)═CH—CH═CH—CH₃, —CH═C(CH₃)—C(CH₃)═CH₂, —C(CH₃)═CH—C(CH₃)═CH₂, —C(CH₃)═C(CH₃)—CH═CH₂, —CH═CH—CH═CH—CH═CH₂, —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —CH₂—CH(CH₃)—C≡CH, —CH(CH₃)—CH₂—C≡CH, —CH(CH₃)—C≡C—CH₃, —C₄H₈—C≡CH, —C₃H₆—C≡C—CH₃, —C₂H₄—C≡C—C₂H₅, —CH₂—C≡C—C₃H₇, —C≡C—C₄H₉, —C₂H₄—CH(CH₃)—C≡CH, —CH₂—CH(CH₃)—CH₂—C≡CH, —CH(CH₃)—C₂H₄—C≡CH, —CH₂—CH(CH₃)—C≡C—CH₃, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —C≡C—CH₂—CH(CH₃)₂, —C≡C—C(CH₃)₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —C(CH₃)(C₂H₅)—C≡CH, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —CH₂—C≡C—C≡C—CH₃, —C≡C—CH₂—C≡C—CH₃, —C≡C—C≡C—C₂H₅, —C≡C—CH(CH₃)—C≡CH, —CH(CH₃)—C≡C—C≡CH, —CH(C≡CH)—CH₂—C≡CH, —C(C≡CH)₂—CH₃, —CH₂—CH(C≡CH)₂, —CH(C≡CH)—C≡C—CH₃.

Particularly preferred are substituents chosen from the group of phenols, methylaniline, dimethylaniline, methyl-2-aminopyridine, dimethyl-2-aminopyridine.

All compounds used and utilizable according to the present invention of the group of arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-denzothiazol-2-yl)phenyl]hydrazones and/or diaryl ureas possess at least 2 and advantageously three aromatic rings which are bound together either directly or via a vinyl bridge or a urea bridge. Particularly preferred are compounds with three aromatic rings which are bound together via a vinyl bridge, which results in the formation of extended π-electron systems. Particularly preferred are compounds of the aforementioned classes with delocalized electrons extending over at least 15 involved atoms. Furthermore preferred are compounds with π-electron systems extending over at least 20 atoms and particularly preferred are delocalized electron systems extending over 22 and more atoms.

In 4,6-divinylpyrimidines, 3,6-divinylpyridazines and in 2,5-divinylpyrazines, 22 atoms are involved to form the π-electron system if Ar represents a 6-member ring structure. The π-electron system extends over 20 atoms if Ar is a 5-member ring structure.

The abovementioned compounds can be used as fluorescent probes for the diagnosis of neurodegenerative disorders. Included herein are all kinds of disorders which lead to a progressing loss of neurons. The classification of neurodegenerative disorders is both based on the clinical manifestation with typical topographic distribution and involved cell type of the degenerative process and on the deposition of structurally modified proteins like prion protein. Tau, beta-amyloid, alpha-synuclein, TDP-43 or Huntington. The fluorescence of compounds of the present invention is either enhanced or significantly shifted upon binding to the target proteins, thus providing the required signal-to-noise ratio.

The term “diagnostics or diagnosis” thereby includes the ranges of in vivo, in vitro, ex vivo diagnostics. Diagnostics or diagnosis generally exclusively or mainly serves the purpose to provide information. This information gives insight into:
physiological or pathological states or
congenital anomalies or
serve to asses safety and compatibility in potential recipients and/or
to monitor therapeutic measures.

In in vitro or ex vivo diagnostics, samples deriving from the human body are used like e.g. blood, serum, plasma, seminal fluid, spinal fluid, peritoneal fluid, saliva, sputum, tear fluid, urine, biopsy material and tissue donations. Gaining such a sample can, but not necessarily has to be part of a diagnostic method. In certain embodiments of the present invention gaining of the designated sample for diagnosis is not a step of the inventive diagnostic method.

In in vivo diagnostics, compounds of this invention are detected after binding to Aβ protein, α-synuclein or Tau-PHF aggregates in the olfactory epithelium and/or bulbus olfactorius.

The utilization of arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones and diaryl ureas is particularly advantageous for the diagnoses of neurodegenerative disorders.

In a particularly preferred embodiment, said arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones and diaryl ureas are detected in the olfactory epithelium and/or bulbus olfactorius.

The invention in particular comprises a procedure for the diagnosis of neurodegenerative disorders, including:
a) Administration of a compound chosen from the group of arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones or diaryl ureas and
b) diagnosis of the neurodegenerative disease in the olfactory epithelium and/or bulbus olfactorius.

The invention furthermore includes a procedure for the in vivo detection of Aβ protein, α-synuclein or Tau-PHF aggregates, comprising:
a) Administration of a compound chosen from the group of arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones or diaryl ureas and
b) detection of Aβ proteins, α-synucleins or Tau-PHF aggregates in the olfactory epithelium and/or bulbus olfactorius.

The herein mentioned compounds act as preferably fluorescent probes, having affinity and preferably high affinity for Aβ protein, α-synuclein or for Tau-PHF aggregates which they bind to in an advantageously specific manner. In this context, specific binding means that a detectable optical response is generated upon binding of the herein disclosed compounds to one or more of the abovementioned target proteins. The increase in extinction upon binding to the target protein is advantageously characterized by a >10× increased improvement of the signal-to-noise ratio as compared to the free compound and can be determined experimentally e.g. on the basis of the reduction of the background noise. Furthermore preferred are compounds having an extinction coefficient of ε>10.000 L·mol⁻¹·cm⁻¹. The determination of the extinction coefficient is carried out at 25° C., pH 7, the respective absorption maximum of the compound and with DMSO as solvent.

The difference between excitation maximum and emission maximum is referred to as Stokes shift. This value essentially determines how well a compound is suited for a use in fluorescence investigations. The higher the Stokes shift, the easier is the experimental discrimination between emission and excitation. Compounds of this invention are advantageously characterized by a Stokes shift >20 nm.

Particularly preferred are furthermore compounds which have an in vivo half life of >60 min. For this purpose, retention time and excretion rate of accordingly labeled probes (e.g. ³H, ¹¹C, ¹⁸F is determined.

The affinity of fluorescent probes is generally determined indirectly by measuring their ability to displace a fluorescent of radioactive reference ligand. In one embodiment, the affinity of ligands of the present invention is characterized by a displacement of thioflavin S, thioflavin T or 11C-PIB with an EC50<300 nM. This can e.g. be measured as described in Lockhart et al., Mar. 4, 2005, The Journal of Biological Chemistry, 280, 7677-7684 under Material & Methods, in particular in the two sections “Radioligand Competition Assay” and “Fluorescence Competition Assay”.

Preferred are compounds which are characterized by increased potential brain permeability and reduced, binding to white matter as well as a reduced plasma protein binding capacity.

The ability of a compound to diffuse though the endothelia of the blood-brain barrier is mainly determined by its fat solubility (lipophilic properties) and its size. In a preferred embodiment, compounds of this invention have a molecular mass of <500 g/mol.

The log P value and log D value are model values for the ratio between lipophilic properties (fat solubility) and hydrophilic properties (water solubility) of a substance. The expectation is that, using the octanol-water partition coefficient, also the partition coefficient of this substance in other systems with aqueous and lipophilic phase can be estimated. A log P value is greater than one if a substance is more soluble in fat-like solvents like n-octanol, and smaller than one if the substance is more soluble in water. The log P value is accordingly positive for lipophilic and negative for hydrophilic substances. Preferred are thus compounds which have a log P value between 1 and 2.8. In further embodiments, compounds with a log D value <5 are preferred. Measurements of log P value and log D value, respectively, are carried out using an octanol/water 2-phase system and UV/VIS spectroscopy at 25° C. and pH 7. Measurements of the log P value or log D value are however not possible for all chemicals. In this case, other models for a prediction can be applied, e.g. by quantitative structure activity relationships (QSAR) or by linear free energy relationships (LFER).

The potential brain permeability of compounds can also be defined using the topological polar surface area (TPSA). This is defined as the sum of surface contributions of polar atoms (in general oxygen atoms, nitrogen and hydrogen atoms) in a molecule. The calculation was among others described by Ertl, P. et al., Fast calculation of molecular polar surface area as a sum of fragment based contributions and its application to the prediction of drug transport properties, J. Med. Chem., 2000, 43, 3714-3717. Preferred are thus in particular compounds having a TPSA<70 Å².

Lack of charges and a low basicity of compounds support a good penetration of the blood-brain-barrier. The inventive compounds are furthermore characterized by a good photostability (low photobleaching) and by a short-lived singlet excitation in contrast to a long-lived triplet excitation.

In further embodiments, compounds of the present invention possess one or more of the following physico-chemical properties. Particularly preferred are compounds which have at least three of the following properties a)-f):
a) a >10fold extinction increase upon binding to the Aβ protein, α-synuclein or to Tau-PHF aggregates as compared to the free compound,
b) a Stokes shift of >20 nm,
c) an extinction coefficient of ε>10 000 L·mol⁻·cm⁻¹,
d) EC50<300 nM
e) a log P value between 1 and 2.8,
f) a topological polar surface area (TPSA)<70 Å².

Preferred are furthermore embodiments in which the inventive compounds possess at least three of the following properties a)-f), whereby at least one is chosen from properties e)-f):
a) a >10fold extinction increase upon binding to the Aβ, protein, α-synuclein or to Tau-PHF aggregates as compared to the free compound,
b) a Stokes shift of >20 nm,
c) an extinction coefficient of ε>10 000 L·mol⁻¹·cm⁻¹,
d) EC50<300 nM
e) a log P value between 1 and 2.8,
f) a topological polar surface area (TPSA)<70 Å².

Furthermore preferred are embodiments in which the inventive compounds possess at least three of the following properties a)-g), whereby at least one is selected from properties e)-g):
a) a >10fold extinction increase upon binding to the Aβ protein, α-synuclein or to Tau-PHF aggregates as compared to the free compound,
b) a Stokes shift of >20 nm,
c) an extinction coefficient of ε>10 000 L·mol⁻¹·cm⁻¹,
d) EC50<300 nM
e) a log P value between 1 and 2.8,
f) a topological polar surface area (TPSA)<70 Å².
g) a log D value <3.

Equally preferred are embodiments in which the inventive compounds possess at least three of the following properties a)-f), whereby at least one is selected from properties a) and d):
a) a >10fold extinction increase upon binding to the Aβ, protein, α-synuclein or to Tau-PHF aggregates as compared to the free compound,
b) a Stokes shift of >20 nm,
c) an extinction coefficient of β>10 000 L·mol⁻¹·cm⁻¹,
d) EC50<300 nM
e) a log P value between 1 and 2.8,
f) a topological polar surface area (TPSA)<70 Å².

In a particularly preferred embodiment, compounds of the present invention possess at least three of the following properties a)-i):
a) a >10fold extinction increase upon binding to the Aβ protein, α-synuclein or to Tau-PHF aggregates as compared to the free compound,
b) a Stokes shift of >20 nm,
c) an extinction coefficient of ε>10 000 L·mol⁻¹·cm⁻¹,
d) EC50<300 nM
e) a log P value between 1 and 2.8,
f) a topological polar surface area (TPSA)<70 Å².
g) a molecular mass <500 g/mol,

h) an in vivo half-life of >60 min,

i) a log D value <3.

The herein disclosed compounds are particularly advantageous for the early diagnosis of neurodegenerative disorders of the group of tauopathies. The group of tauopathies comprises neurodegenerative disease patterns which share the common feature of Tau-protein accumulation in the brain. The Tau-protein is a low-molecular weight protein which binds to cellular cytoskeleton proteins (microtubuli) and regulate the assembly thereof.

In a preferred embodiment, the compounds referred to herein are utilized for an early diagnosis of tauopathies like e.g. Alzheimer's disease, corticobasal degeneration, argyrophilic grain disease, Pick's disease, FTDP-17 (frontotemporal dementia and parkinsonism of chromosome 17) or progressive supranuclear palsy.

According to the present invention, compounds are furthermore advantageous for the early diagnosis of neurodegenerative disorders of the group of synucleinopathies. The group of synucleinopathies comprises neurodegenerative disease patterns which all share the common feature of α-synuclein protein accumulation in the brain like e.g. Parkinson's disease. The α-synuclein protein is a protein of 140 amino acids which is normally only present in the presynaptic regions of axons. Alpha-synuclein is a main component of neuronal intracellular protein aggregates (=Levy-bodies), the characteristic neuropathological feature of synucleinopathies.

Diagnostic compositions containing at least one compound of the group of arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones or diaryl ureas are administered in an effective dose to elicit an optical response which is detectable in imaging diagnostics. A detectable optical response is characterized in that an optical signal occurs or changes, which can be observed or measured with suitable devices. In certain embodiments this optical response is in most cases related to a change in fluorescence like e.g. a change in intensity, excitation or emission wave length, changes in fluorescence lifetime or fluorescence polarization.

Compounds of the group of arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones and diaryl ureas can be administered systemically or locally. In one embodiment, compounds are administered intravenously. In another embodiment, the fluorescent probes are administered parenterally. In a further embodiment, compounds are administered enterally. In a preferred embodiment, compounds are administered orally. In a further embodiment, compounds are administered topic nasally. Compositions with compounds utilized according to this invention typically include an effective concentration of said compounds in aqueous solution or suspension which in addition may also contain buffers, surfactants, thixotroping agents, cosolvents, flavoring agents and the like.

Compounds as referred to herein are furthermore preferably able to pass the blood-brain barrier. In further embodiments, compounds utilized according to this invention are able to pass the blood-tissue barrier, the blood-liver barrier, the blood-liquor barrier, the liquor-brain barrier, the blood-nerve barrier and/or the placental barrier.

In addition to the brain, the disease-causing protein deposits can also be found in the olfactory epithelium and/or bulbus olfactoris.

In a preferred embodiment, compounds as disclosed in the present application are detected after binding to Aβ protein, α-synuclein or to Tau-PHF aggregates in the olfactory epithelium and/or bulbus olfactoris of the patient. The detection is performed using an adaption of fiber optics or fluorescence microscopy.

Detection is carried out using suitable filter systems or detectors which are known to the state of the art. Preferred is an excitation in the wavelength-range 380-900 nm and an emission between 400-1000 nm. Particularly preferred is an excitation between 450-500 nm and an emission at 600-650 nm and 600-700 nm.

In a further embodiment, Alzheimer's disease is diagnosed via the confirmed absence of Tau-aggregates in the intestinal epithelium. The presence of Tau-aggregates in the intestinal epithelium is inversely correlated with the diagnosis of Alzheimer's disease. If no Tau-aggregates are detected with herein disclosed compounds, is this a reliable indication for Alzheimer's disease.

Preferred is the utilization of said compounds of arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones or diaryl ureas for the manufacture of a diagnostic agent for the diagnosis of neurodegenerative disorders.

Confirming the presence of the aforementioned aggregates in the olfactory epithelium and/or bulbus olfactoris it is for the first time possible to detect neurodegenerative diseases in a non-invasive manner and ante mortem using a fast and simple detection procedure. The use of said compounds as fluorescent probes furthermore has the advantage that the equipment required and consequently also the costs are reduced substantially.

In particularly preferred embodiments, compounds of the present invention are the following compounds:

### 4-((1E)-2-(6-(4-(dimethylamino)styryl)pyrimidine-4-yl)vinyl)-N,N-dimethylbenzenamine

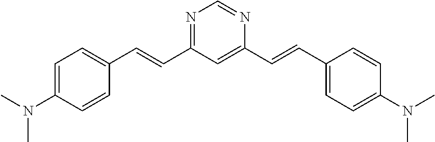

### 4,4′-(1E,1′E)-2,2′-(2-(methylthio)pyrimidine-4,6-diyl)bis(ethene-2,1-diyl)bis(N,N-dimethylaniline)

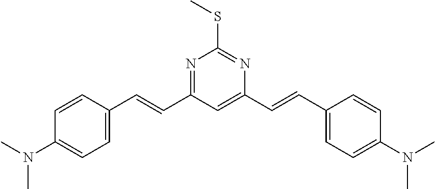

### 4,6-Bis((E)-2-(1-methyl-1H-pyrrol-2-yl)vinyl)pyrimidine

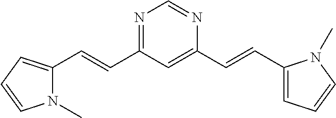 [SMILES: Cn1cccc1/C=C/c1cc(/C=C/c2cccn2C)ncn1]

### 4,6-Bis((E)-2-(naphthalene-1-yl)vinyl)pyrimidine

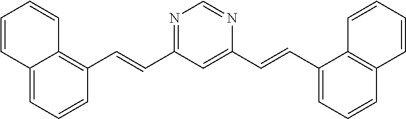 [SMILES: C(=C\c1cccc2ccccc12)/c1cc(/C=C/c2cccc3ccccc23)ncn1]

### 4,4′-(1E,1′E)-2,2′-(pyrazine-2,5-diyl)bis(ethene-2,1-diyl)bis(N,N-dimethylaniline)

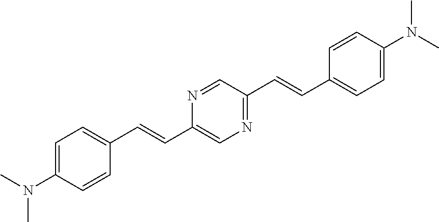

### 2,6-Bis((E)-2-(1-methyl-1H-pyrrol-2-yl)vinyl)pyrazine

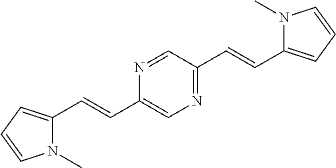 [SMILES: Cn1cccc1/C=C/c1cnc(/C=C/c2cccn2C)cn1]

### 2,5-Bis(4-methoxystyryl)pyrazine

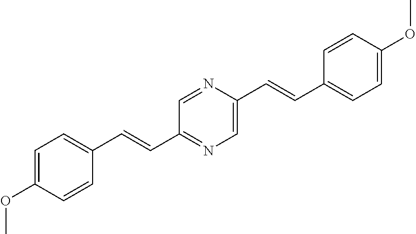 [SMILES: COc1ccc(/C=C/c2cnc(/C=C/c3ccc(OC)cc3)cn2)cc1]

### (Z)-1-(4-(benzo[d]thiazol-2-yl)phenyl)-2-((9-methyl-9H-carbazol-3-yl)methylene)hydrazine

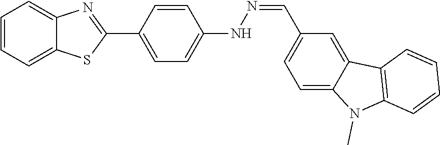 [SMILES: Cn1c2ccccc2c2cc(/C=N\Nc3ccc(-c4nc5ccccc5s4)cc3)ccc21]

### (Z)-1-(4-(benzo[d]thiazol-2-yl)phenyl)-2-((pyridine-3-yl)methylene)hydrazine

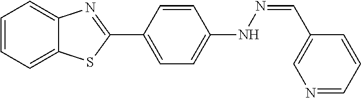 [SMILES: C(=N/Nc1ccc(-c2nc3ccccc3s2)cc1)/c1cccnc1]

### N′-(4-(7-(diethylamino)-2-oxo-2H-chromen-3-yl)thiazol-2-yl)nicotinohydrazide

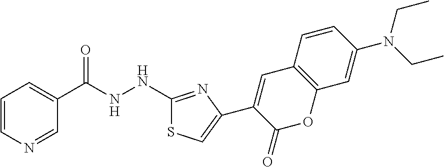 [SMILES: CCN(CC)c1ccc2cc(-c3csc(NNC(=O)c4cccnc4)n3)c(=O)oc2c1]

### 1-(Benzo[c][1,2,5]oxadiazol-5-yl)-3-(3-fluorobenzyl)urea

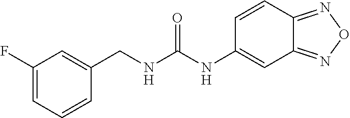 [SMILES: O=C(NCc1cccc(F)c1)Nc1ccc2nonc2c1]

### 4,4′-(1E,1′E)-2,2′-(Pyrimidine-4,6-diyl)bis(1-methylethen-2,1-diyl)bis(N,N-dimethylaniline)

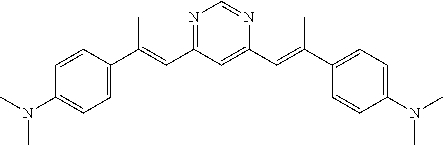

### 4,4′-(1E,1′E)-2,2′-(5-Methylpyrimidine-4,6-diyl)bis(ethen-2,1-diyl)bis(N,N-dimethylaniline)

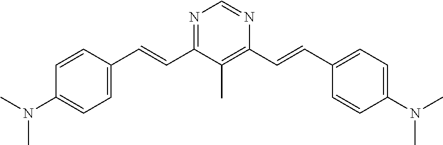

### 4,4′-(1E,1′E)-2,2′-(Pyrimidine-4,6-diyl)bis(ethen-2,1-diyl)bis(N,N,3,5-tetramethyl-aniline)

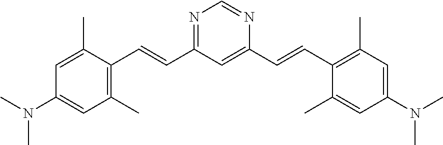

### 4,4′-(1E,1′E)-2,2′-(Pyrimidine-4,6-diyl)bis(2-methylethen-2,1-diyl)bis(N,N-dimethylaniline)

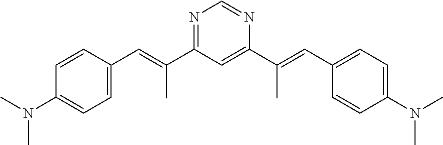

### 2-[3-(1,3-Dihydro-1,3,3-trimethyl-2H-indol-2-yliden)-1-propen-1-yl]-1,3,3-trimethyl-3H-indolium Iodide

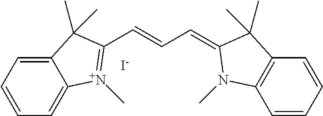 [SMILES: CN1/C(=C\C=C\C2=[N+](C)c3ccccc3C2(C)C)C(C)(C)c2ccccc21 ; [I-]]

### 2-[3-(1,3-Dihydro-1-{2-[2-(2-methoxyethoxy)ethoxy]ethyl}-3,3-dimethyl-2H-indol-2-yliden)-1-propen-1-yl]-1-{2-[2-(2-methoxyethoxy)ethoxy]ethyl}-3, -3H-indolium Chloride

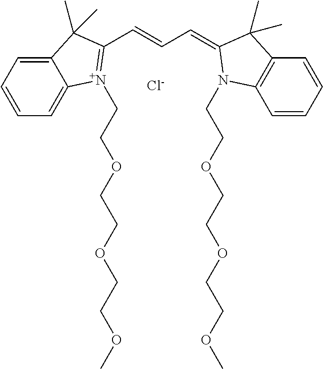 [SMILES: COCCOCCOCCN1/C(=C\C=C\C2=[N+](CCOCCOCCOC)c3ccccc3C2(C)C)C(C)(C)c2ccccc21 ; [Cl-]]

### -3H-indolium Chloride

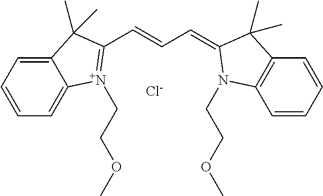 [SMILES: COCCN1/C(=C\C=C\C2=[N+](CCOC)c3ccccc3C2(C)C)C(C)(C)c2ccccc21 ; [Cl-]]

### 2-[3-(1,3-Dihydro-3,3-dimethyl-1-butyl-2H-indol-2-yliden)-1-propen-1-yl]-3,3-dimethyl-1-butyl-indolium Iodide

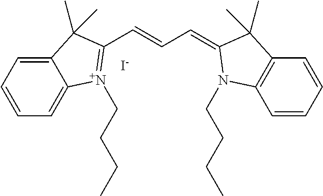 [SMILES: CCCCN1/C(=C\C=C\C2=[N+](CCCC)c3ccccc3C2(C)C)C(C)(C)c2ccccc21 ; [I-]]

### 5-Nitro-2-[3-(5-nitro-1,3-dihydro-1,3,3-trimethyl-2H-indol-2-yliden)-1-propen-1-yl]-1,3,3-trimethyl-3H-indolium Iodide

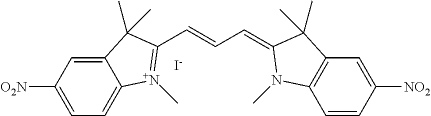 [SMILES: CN1/C(=C\C=C\C2=[N+](C)c3ccc([N+](=O)[O-])cc3C2(C)C)C(C)(C)c2cc([N+](=O)[O-])ccc21 ; [I-]]

### 2-[3-(1,3-Dihydro-3,3-dimethyl-1-octyl-2H-indol-2-yliden)-1-propen-1-yl]-3,3-dimethyl-1-octyl-indolium Bromide

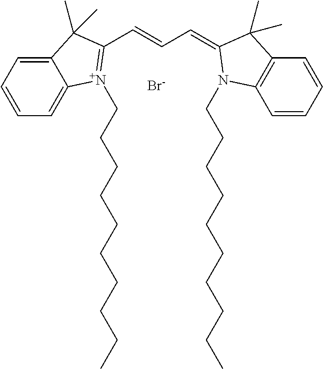 [SMILES: CCCCCCCCCCN1/C(=C\C=C\C2=[N+](CCCCCCCCCC)c3ccccc3C2(C)C)C(C)(C)c2ccccc21 ; [Br-]]

### 5-Bromo-2-[3-(5-bromo-1,3-dihydro-1,3,3-trimethyl-2H-indol-2-yliden)-1-propen-1-yl]-1,3,3-trimethyl-3H-indolium Iodide

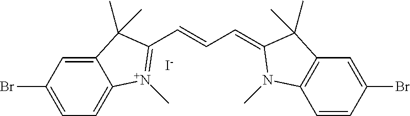 [SMILES: CN1/C(=C\C=C\C2=[N+](C)c3ccc(Br)cc3C2(C)C)C(C)(C)c2cc(Br)ccc21 ; [I-]]

### 3-Methyl-2-[3-(3-methyl-2(3H)-benzothiazolyliden)-1-propenyl]-benzothiazolium Iodide

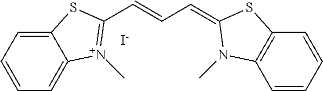 [SMILES: CN1/C(=C\C=C\c2sc3ccccc3[n+]2C)Sc2ccccc21 ; [I-]]

### EXAMPLES

### Example 1

### BSc4090: 4-((1E)-2-(6-(4-(dimethylamino)styryl)pyrimidine-4-yl)vinyl)-N,N-dimethylbenzenamine

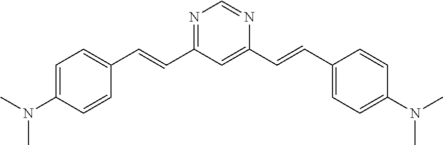

Synthesis: 4,6-Dimethylpyrimidine (100 mg, 0.92 mmol), 4-(dimethylamino)benzaldehyde (275 mg, 1.85 mmol) and aliquat 336 (13 mg, 0.03 mmol) are dissolved in 5M NaOH-solution (10 ml). The solution is heated to boiling point for 1 h at 110° C., subsequently stirred for 3 h at room temperature. The solution is filtered and the resulting solid is re-crystallized from methanol (15 ml). Obtained are 65 mg (20%) of BSc4090 as yellow solid.

¹H-NMR (CDCL₃, 500 MHz): δ=8.90 (s, 1H), 7.73 (d, J=15.8 Hz, 2H), 7.42 (d, J=8.6 Hz, 4H), 7.06 (s, 1H), 6.76 (d, J=15.8 Hz, 2H), 6.62 (d, J=8.6 Hz, 4H), 2.93 (s, 12H) ppm.

¹³C-NMR (CDCl₃, 125 MHz): δ=163.4, 158.5, 151.5, 137.6, 129.5, 124.2, 121.2, 115.4, 112.4, 40.6 ppm.

MS (EI) m/z=370 (M⁺), 326, 283

### Example 2

### BSc4097: 4,4′-(1E,1′E)-2,2′-(2-(methylthio)pyrimidine-4,6-diyl)bis(ethene-2,1-diyl)bis(N,N-dimethylaniline)

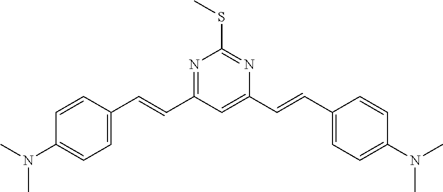

Synthesis: 4,6-Dimethylpyrimidine-2-thiol (100 mg, 0.64 mmol), 4-(dimethylamino)benzaldehyde (193.5 mg, 1.29 mmol) and aliquat 336 (25 mg, 0.06 mmol) are dissolved in 5M NaOH-solution (10 ml). The solution is heated to boiling point for 1 h at 110° C., subsequently stirred for 3 h at room temperature. The solution is filtered and the resulting solid is re-crystallized from methanol. Obtained are 175 mg BSc4097 (65%) as yellow solid.

¹H-NMR (CDCL₃, 300 MHz): δ=7.80 (d, J=15.8 Hz, 1H), 7.49 (d, J=8.8 Hz, 4H), 6.83 (d, J=6.9 Hz, 2H), 6.77 (s, 1H), 6.70 (d, J=8.8 Hz, 4H), 3.01 (s, 12H), 2.67 (s, 3H) ppm.

¹³C-NMR (CDCl₃, 125 MHz): δ=171.2, 163.1, 151.0, 137.1, 129.1, 124.0, 121.1, 112.1, 110.7, 40.2, 14.2 ppm.

MS (EI) m/z=416 (M⁺), 401, 369, 326

### Example 3

### BSc4327: 4,6-Bis((E)-2-(1-methyl-1H-pyrrol-2-yl)vinyl)pyrimidine

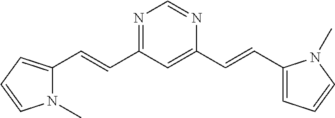 [SMILES: Cn1cccc1/C=C/c1cc(/C=C/c2cccn2C)ncn1]

Synthesis: 4,6-Dimethylpyrimidine (150 mg, 1.38 mmol), 1-Methyl-1H-pyrrole-2-carbaldehyde (302.3 mg, 2.77 mmol) and aliquat 336 (55 mg, 0.13 mmol) are dissolved in 5M NaOH-solution (15 ml). The solution is heated to boiling point for 1 h at 110° C., subsequently stirred for 3 h at room temperature. The solution is filtered and purified by column chromatography (Axel Semrau FlashMaster Cy/EE gradient). Obtained are 162 mg BSc4327 (40%) as yellow solid.

¹H-NMR (CDCL_{3,} 300 MHz): δ=8.98 (s, 1H), 7.86 (d, J=15.5 Hz, 2H), 7.00 (s, 1H), 6.73 (q, m, 6H), 6.21 (m, 2H), 3.77 (s, 6H) ppm.

¹³C-NMR (CDCl₃, 125 MHz): δ=162.7, 158.5, 130.8, 125.9, 124.4, 121.4, 116.1, 110.0, 109.1, 108.3, 34.3 ppm.

MS (ESI) m/z=289.2 (M⁺⁽⁻¹⁾), 275.2,

### Example 4

### BSc4328: 4,6-Bis((E)-2-(naphthalene-1-yl)vinyl)pyrimidine

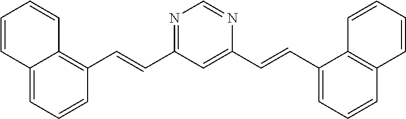 [SMILES: C(=C\c1cccc2ccccc12)/c1cc(/C=C/c2cccc3ccccc23)ncn1]

Synthesis: 4,6-Dimethylpyrimidine (100 mg, 0.92 mmol), 1-naphthaldehyde (287.4 mg, 1.84 mmol) and aliquat 336 (37 mg, 0.09 mmol) are dissolved in 5M NaOH-solution (15 ml). The solution is heated to boiling point for 1 h at 110° C., The solution is filtered and purified by column chromatography (Axel Semrau FlashMaster Cy/EE gradient). Obtained are 110 mg BSc4328 (40%) as green-yellow solid.

¹H-NMR (CDCL₃, 300 MHz): δ=9.12 (s, 1H), 8.69 (d, J=15.6 Hz, 2H), 8.24 (d, J=8.2 Hz, 2H), 7.78 (m, 6H), 7.45 (m, 6H), 7.24 (s, 1H), 7.06 (d, J=15.6 Hz, 2H) ppm.

¹³C-NMR (CDCl₃, 125 MHz): δ=162.8, 158.8, 134.1, 133.7, 133.2, 131.5, 129.7, 128.5,000, 126.6, 126.1, 125.5, 124.5 13.7, 116.9 ppm.

MS (EI) m/z=383 (M⁺⁽⁻¹⁾), 275.2,

### Example 5

### BSc4352: 4,4′-(1E,1′E)-2,2′-(pyrazine-2,5-diyl)bis(ethene-2,1-diyl)bis(N,N-dimethylaniline)

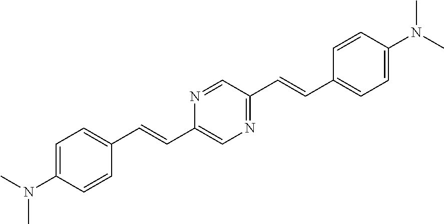

Synthesis: 2,5-Dimethylpyrazine (0.125 g, 1.16 mmol) is dissolved in 10 ml dimethylformamide, followed by addition of 4-(dimethylamino)benzaldehyde (0.345 g, 2.32 mmol) and potassium-t-butoxide (0.26 g, 2.32 mmol). The solution is heated for 4 h to 80° C. and allowed to cool to room temperature. In this process, a solid crystallizes out. The solution containing the crystallized solid is filtered and washed with ethyl acetate. After drying under high-vacuum, 0.308 g (yield: 72%) of the product BSc4352 is obtained as red solid.

¹H-NMR (CDCl₃, 500 MHz): δ=8.49 (s, 2H), 7.62 (d, J=16 Hz, 2H), 7.49 (d, J=8.9 Hz, 4H), 6.96 (d, J=16 Hz, 2H), 6.72 (d, J=8.9 Hz, 4H), 3.02 (s, 12H) ppm.

¹³C-NMR (CDCl₃, 125 MHz): δ=149.7, 148.0, 141.7, 132.8, 127.5, 123.7, 118.7, 111.2, 39.3 ppm.

### Example 6

### BSc4353: 2,5-Bis((E)-2-(1-methyl-1H-pyrrol-2-yl)vinyl)pyrazine

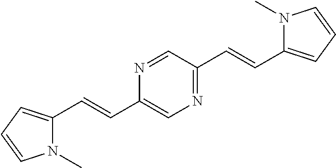 [SMILES: Cn1cccc1/C=C/c1cnc(/C=C/c2cccn2C)cn1]

Synthesis: 2,5-Dimethylpyrazine (0.125 g, 1.16 mmol) is dissolved in 10 ml dimethylformamide, followed by addition of 1-methyl-1H-pyrrol-2-carbaldehyde (0.252 g, 2.32 mmol) and potassium-t-butoxide (0.26 g, 2.32 mmol), and heated for 4 h to 80° C. and allowed to cool to room temperature. In this process, a solid crystallizes out. The solution containing the crystallized solid is filtered and washed with ethyl acetate. After drying under high-vacuum, 0.215 g (yield: 64%) of the product BSc4353 is obtained as orange-red solid.

¹H-NMR (CDCl_{3,} 500 MHz): δ=8.40 (s, 2H), 7.62 (d, J=15.6 Hz, 2H), 6.88 (d, J=15.6 Hz, 2H), 6.70 (m, 2H), 6.65 (m, 2H), 6.19 (m, 2H), 3.76 (s, 6H) ppm.

¹³C-NMR (CDCl₃, 125 MHz): δ=147.8, 142.1, 130.5, 124.1, 120.6, 119.3, 107.9, 107.8, 33.3 ppm.

### Example 7

### BSc4354: 2,5-Bis(4-methoxystyryl)pyrazine

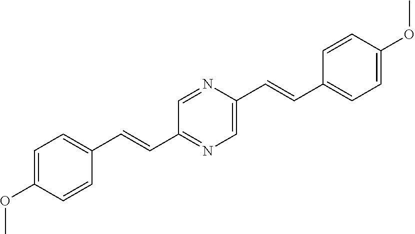 [SMILES: COc1ccc(/C=C/c2cnc(/C=C/c3ccc(OC)cc3)cn2)cc1]

Synthesis: 2,5-Dimethylpyrazine (0.125 g, 1.16 mmol) is dissolved in 10 ml dimethylformamide, followed by addition of 4-methoxybenzaldehyde (0.315 g, 2.32 mmol) and potassium-t-butoxide (0.26 g, 2.32 mmol) The solution is heated for 4 h to 80° C. and allowed to cool to room temperature. In this process, a solid crystallizes out. The solution containing the crystallized solid is filtered and washed with ethyl acetate. After drying under high-vacuum, 0.370 g (yield: 93%) of the product BSc4354 is obtained as yellow solid.

¹H-NMR (CDCl₃, 500 MHz): δ=8.54 (s, 2H), 7.67 (d, J=16 Hz, 2H), 7.55 (d, J=9 Hz, 4H), 7.04 (d, J=16 Hz, 2H), 6.93 (d, J=9 Hz, 4H, 3.85 (s, 6H) ppm.

¹³C-NMR (CDCl₃, 125 MHz): δ=158.3, 146.9, 141.0, 131.7, 126.7, 120.0, 112.3, 53.4 ppm.

### Example 8

### BSc4342: (Z)-1-(4-(benzo[d]thiazol-2-yl)phenyl)-2-((9-methyl-9H-carbazol-3-yl)methylene)hydrazin

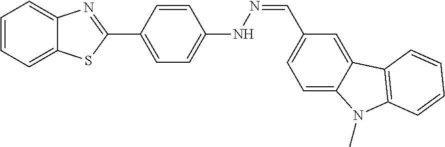 [SMILES: Cn1c2ccccc2c2cc(/C=N\Nc3ccc(-c4nc5ccccc5s4)cc3)ccc21]

Synthesis: To a solution of 4-(benzo[d]thiazol-2-yl)aniline (226 mg, 1 mmol) in 6 N HCl(aq) (1 ml), NaNO₂ (76 mg, 1.1 mmol) in water (1 ml) is added at 0° C. and stirred for 5 min. The resulting solution is added at −5° C. slowly to a solution of SnCl₂ (500 mg, 2.6 mmol) in conc. HCl (aq) (0.5 ml) and subsequently stirred for 1 h at room temperature. 9-Methyl-9H-carbazol-3-carbaldehyde (226 mg, 1 mmol) in tetrahydrofuran (30 ml) and NaOH (3 g, 75 mmol) is added to the reaction mixture and heated for 3 h to reflux. The cooled reaction solution is washed twice with water, dried over Na₂SO₄ and the solvent is removed under vacuum. After column-chromatographic purification (1:1 EtOAc/hexane, silica gel), 291 mg (67.4%) BSc4342 are obtained.

¹H-NMR (500 MHz, DMSO): δ=10.87-10.84 (s, 1H), 8.44-8.42 (s, 1H), 8.26-8.23 (d, J=8.0 Hz, 1H), 8.19-8.16 (s, 1H), 8.08-8.05 (d, J=8.0 Hz, 1H), 7.99-7.95 (d, J=9.0 Hz, 2H), 7.98-7.94 (d, J=8.0 Hz, 1H), 7.94-7.90 (dd, J=8.5 Hz, J=1.5 Hz, 1H), 7.67-7.63 (d, J=8.5 Hz, 1H), 7.63-7.60 (d, J=8.5 Hz, 1H), 7.52-7.47 (m, 2H), 7.40-7.36 (td, J=8.0 Hz, J=1.0 Hz, 1H), 7.28-7.23 (m, 3H), 3.92-3.90 (s, 3H) ppm.

¹³C-NMR (500 MHz, DMSO): δ=167.60, 153.77, 148.09, 140.95, 140.82, 140.39, 133.76, 128.66, 126.36, 126.23, 125.91, 124.47, 123.82, 123.67, 122.51, 122.10, 121.89, 121.55, 120.36, 119.36, 118.69, 111.84, 109.46, 109.30, 29.04 ppm.

MS (EI): m/z=432 (M)+.

### Example 9

### BSc4337: (Z)-1-(4-(benzo[d]thiazol-2-yl)phenyl)-2-((pyridine-3-yl)methylene)hydrazine

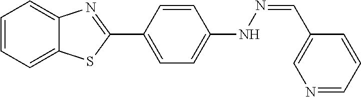 [SMILES: C(=N/Nc1ccc(-c2nc3ccccc3s2)cc1)/c1cccnc1]

Synthesis: To a solution of 4-(benzo[d]thiazol-2-yl)aniline (226 mg, 1 mmol) in 6 N HCl(aq) (1 ml), NaNO₂ (76 mg, 1.1 mmol) in water (1 ml) is added at 0° C. and stirred for 5 min. The resulting solution is added at −5° C. slowly to a solution of SnCl₂ (500 mg, 2.6 mmol) in conc. HCl(aq) (0.5 ml) and subsequently stirred for 1 h at room temperature. 3-Pyridinecarboxaldehyde (107 mg, 1 mmol) in tetrahydrofuran (30 ml) and NaOH (3 g, 75 mmol) is added to the reaction mixture and heated for 3 h to reflux. The cooled reaction solution is washed twice with water, dried over Na₂SO₄ and the solvent is removed under vacuum. After column-chromatographic purification (1:1 EtOAc/hexane, silica gel), 257 mg (77.9%) BSc4337 are obtained.

¹H-NMR (500 MHz, DMSO): δ=11.07-11.05 (s, 1H), 8.86-8.84 (d, J=2.0 Hz, 1H), 8.53-8.50 (dd, J=5.0 Hz, J=2.0 Hz, 1H), 8.13-8.10 (dt, J=8.0 Hz, J=2.0 Hz, 1H), 8.09-8.05 (d, J=8.0 Hz, 1H), 8.00-7.95 (m, 4H), 7.52-7.47 (td, J=8.0 Hz, J=1.0 Hz, 1H), 7.46-7.42 (m, 1H), 7.41-7.37 (td, J=8.0 Hz, J=1.0 Hz, 1H), 7.28-7.23 (d, J=8.5 Hz, 2H) ppm.

¹³C-NMR (500 MHz, DMSO): δ=167.40, 153.70, 149.00, 147.58, 147.28, 135.60, 133.84, 132.24, 131.13, 128.66, 126.28, 124.62, 123.74, 123.58, 122.00, 121.94, 112.30 ppm.

MS (EI): m/z=330 (M)+.

### Example 10

### BSc4007: N′-(4-(7-(diethylamino)-2-oxo-2H-chromen-3-yl)thiazol-2-yl)nicotinohydrazide

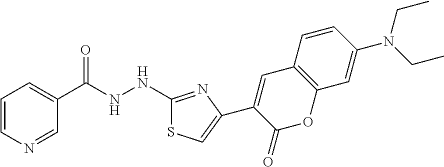 [SMILES: CCN(CC)c1ccc2cc(-c3csc(NNC(=O)c4cccnc4)n3)c(=O)oc2c1]

Synthesis: 3-(2-Carbamothioylhydrazinecarbonyl)pyridinium chloride (0.116 g, 0.5 mmol) is dissolved in approx. 3 ml EtOH, followed by addition of 3-(2-bromoacetyl)-7-(diethylamino)-2H-chromen-2-on (0.169 g, 0.5 mmol) and heating to reflux (30 min). The assay is cooled to room temperature. In this process, a solid (hydrobromide of phenylthiazole benzhydrazide) precipitates. The solution is filtered and the resulting solid is washed with cold EtOH. To convert the raw product into the salt-free form, the solid is dissolved/suspended in EE and washed with sat. NaHCO₃ solution (3×25 ml) and sat. NaCl solution (1×25 ml). The organic phase is dried over sodium sulfate, filtered and the solvent is removed under vacuum. Obtained is 0.150 g (yield: 69%) of the product BSc4007 as yellow solid.

1H-NMR (DMSO, 500 MHz): δ=11.13 (s, NH), 9.75 (s, NH), 9.12 (s, 1H), 8.84 (m, 1H), 8.44 (s, 1H), 8.30 (m, 1H), 7.63 (m, 1H), 7.60 (d, J=8.9 Hz, 1H), 7.53 (s, 1H), 6.77 (m, 1H), 6.61 (s, 1H), 3.50 (q, J=7.1 Hz, 4H), 1.18 (t, J=7.1 Hz, 6H) ppm. 13C-NMR (CDCl3, 125 MHz): δ=171.8, 165.5, 159.9, 155.6, 153.1, 150.9, 148.7, 145.4, 139.8, 135.6, 130.2, 128.5, 124.2, 113.7, 109.8, 108.3, 106.8, 96.5, 45.0, 12.7 ppm.

### Example 11

### BSc4138: 1-(Benzo[c][1,2,5]oxadiazol-5-yl)-3-(3-fluorobenzyl)urea

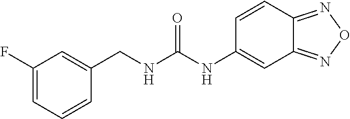 [SMILES: O=C(NCc1cccc(F)c1)Nc1ccc2nonc2c1]

Synthesis: 100 mg (0.529 mmol, 1 eq.) of 2,1,3-benzoxadiazol-5-carboxylic acid azide is dissolved in 3 ml dry DMF. To this solution, 80 mg (0.635 mmol, 1.2 eq.) 4-fluorobenzylamine is added, followed by stirring at 55° C. for 24 h. Water is added to the reaction product and the assay is cooled. Subsequent filtration and washing of the residue with water yield 118 mg (yield 42%) of the product BSc4138 as white solid.

1H-NMR (DMSO, 500 MHz): δ=4.54 (d, J=5.91 Hz, 2H), 7.11 (m, 1H), 7.23 (m, 2H), 7.42 (m, 1H), 7.98 (m, 1H), 8.14 (m, 1H), 8.58 (m, 1H), 9.45 (t, J=5.9 Hz, NH) ppm.

13C-NMR (DMSO, 125 MHz): δ=42.38, 113.60, 113.94, 115.73, 116.32, 123.23, 130.20, 131.31, 137.64, 141.85, 148.74, 149.00, 162.15, 164.59 ppm.

### Example 12

### General Synthesis

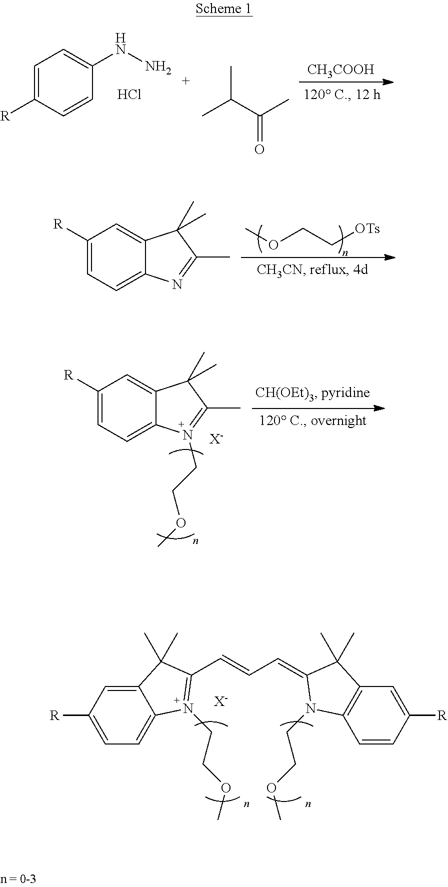

Reaction of p-Toluenesulfonyl Chloride with Polyethylene Glycol

A solution of polyethylene glycol (10 mmol, 1.0 eq.) in THF (3 mL) is added dropwise to a solution of NaOH (13 mmol, 1.3 eq.) in 1 mL water at 0° C. Subsequently p-toluenesulfonyl chloride (11 mmol, 1.1 eq.) is added to the reaction mixture. After stirring for 12 hours at r.t. the reaction is quenched by adding 2.5 ml of water and extracted with DCM (20 mL). The joined organic phases are washed with 2N HCl (15 mL) and sat. aqueous brine (15 mL) and dried over MgSO₄. After filtration the solvent is removed in vacuo.

### 5-Substituted-2,3,3-Trimethylindolenine

A mixture of phenylhydrazine hydrochlorid (1 mmol) and 3-methyl-2-butanone (1.2 mmol) is dissolved in glacial acetic acid (5 mL) and heated under reflux for 12 hours in an argon atmosphere.

After cooling to room temperature the solvent is removed in vacuo and the residue is dissolved in DCM (30 mL), washed with 10% aqueous Na₂CO₃ solution (2×30 mL) and dried over sodium sulfate. After removal of the solvent in vacuo the crude product is purified by column chromatography (SiO₂; ethyl acetate:cyclohexane=1:2) and the product is yielded as yellow liquid.

### 1-PEG-2,3,3-trimethyl-5-substituted-3H-indolium Iodide

5-Substituted-2,3,3-trimethyl-indolenine (5 mmol) and 2-methoxyethyl 4-toluene sulfonate (10 mmol) are dissolved in dry acetonitrile (10 mL). The reaction mixture is heated under reflux for 4 days, wherein the reaction mixture turns purple. It is cooled to r.t. and the crude product is purified by column chromatography (SiO₂; CH₂Cl₂:CH₃OH=10:1) yielding a purple liquid. A solution of 1-PEG-2,3,3-trimethyl-5-substituted-3H-indolium iodide (1 mmol) and triethylorthoformiate (2 mmol) in 1 ml dry pyridine is heated under reflux for 16 hours in an argon atmosphere. After cooling to room temperature the reaction mixture is purified by column chromatography (SiO₂; CH₂Cl₂:CH₃OH=10:1) yielding the final product as waxy solid.

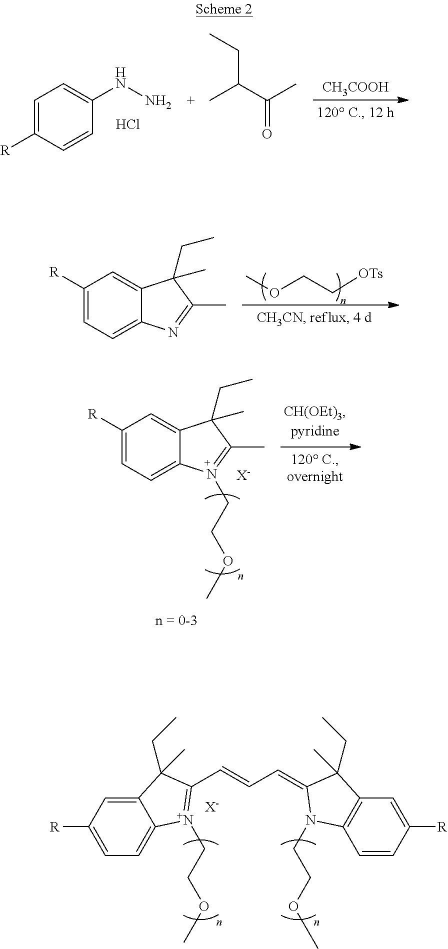

### 5-Substituted-3-ethyl-2,3-dimethylindolenine

A mixture of phenylhydrazine hydrochlorid (1 mmol) and 3-methyl-pentan-2-one (1.2 mmol) is dissolved in glacial acetic acid (5 mL) and heated under reflux for 12 hours in an argon atmosphere. After cooling to room temperature the solvent is removed in vacuo and the residue is dissolved in DCM (30 mL), washed with 10% aqueous Na₂CO₃ solution (2×30 mL) and dried over sodium sulfate. After removal of the solvent in vacuo the crude product is purified by column chromatography (SiO₂; ethyl acetate:cyclohexane=1:2).

### 1-PEG-3-ethyl-2,3-dimethyl-5-substituted-3H-indolium Iodide

5-Substituted-3-ethyl-2,3-dimethylindolenine (5 mmol) and 2-methoxyethyl 4-toluene sulfonate (10 mmol) are dissolved in dry acetonitrile (10 mL). The reaction mixture is heated under reflux for 4 days wherein the reaction mixture turns purple. It is cooled to r.t. and the crude product is purified by column chromatography (SiO₂; CH₂Cl₂:CH₃OH=10:1) yielding 1-PEG-3-ethyl-2,3-dimethyl-5-substituted-3H-indolium iodide. A solution of 1-PEG-3-ethyl-2,3-dimethyl-5-substituted-3H-indolium iodide (1 mmol) and triethylorthoformiate (2 mmol) in 1 ml dry pyridine is heated under reflux for 16 hours in an argon atmosphere. After cooling to room temperature the reaction mixture is purified by column chromatography (SiO₂; CH₂Cl₂:CH₃OH=10:1) yielding the final product.

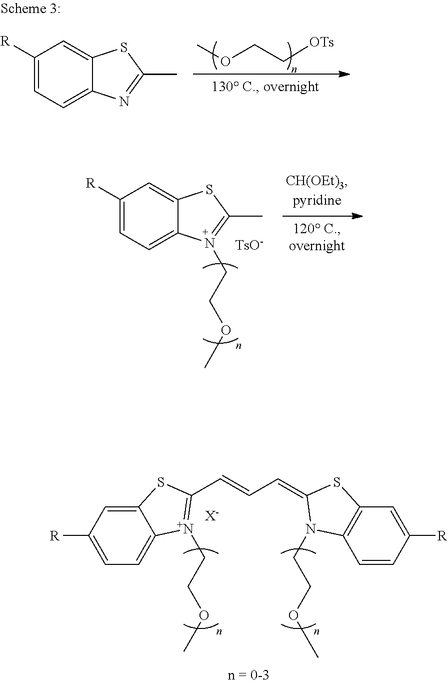

### 6-Substituted 1-methyl-2-PEG-benzo[d]thiazol-3-ium

In a 10 ml flask 2-methoxyethyl-4-toluenen sulfonate (6 mmol) is added to 6-substituted 2-methylbenzo[d]thiazol (5 mmol). The reaction mixture is heated to 120° C. in an argon atmosphere and stirred for 12 hours, wherein the reaction turns purple. It is cooled to r.t. The precipitating solid is re-crystallized from ethyl acetate and 6-substituted 1-methyl-2-PEG-benzo[d]thiazol-3-ium is yielded as colorless solid.

A solution of 6-substituted 1-methyl-2-PEG-benzo[d]thiazol-3-ium (1 mmol) and triethylorthoformiate (2 mmol) in 1 ml dry pyridine is heated under reflux for 16 hours in an argon atmosphere. After cooling to room temperature the reaction mixture is purified by column chromatography (SiO₂; CH₂Cl₂:CH₃OH=10:1) yielding the final product.

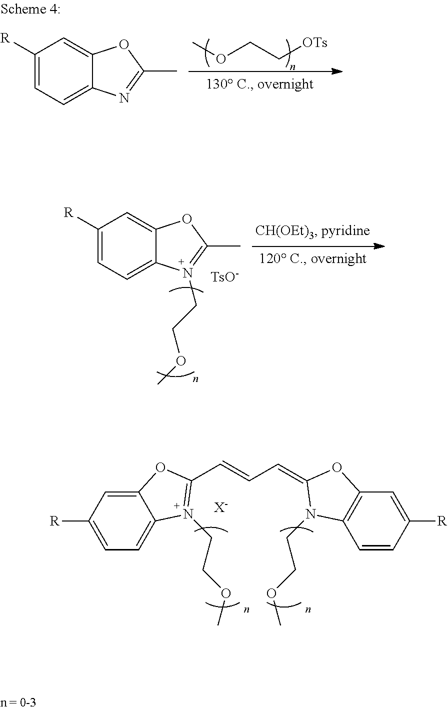

### 6-Substituted 1-methyl-2-PEG-benzo[d]thiazol-3-ium

In a 10 ml flask 2-methoxyethyl-4-toluenen sulfonate (6 mmol) is added to 6-substituted 2-methylbenzo[d]oxazol (5 mmol). The reaction mixture is heated to 120° C. in an argon atmosphere and stirred for 12 hours, wherein the reaction turns purple. It is cooled to r.t. The precipitating solid is re-crystallized from ethyl acetate and 6-substituted 1-methyl-2-PEG-benzo[d]oxazol-3-ium is yielded as colorless solid.

A solution of 6-substituted 1-methyl-2-PEG-benzo[d]oxazol-3-ium (1 mmol) and triethylorthoformiate (2 mmol) in 1 ml dry pyridine is heated under reflux for 16 hours in an argon atmosphere. After cooling to room temperature the reaction mixture is purified by column chromatography (SiO₂; CH₂Cl₂:CH₃OH=10:1) yielding the final product.

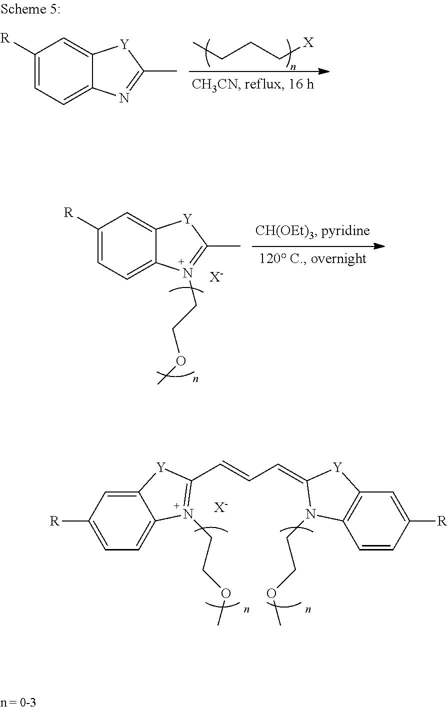

The corresponding starting material (5 mmol) and the halogenalkane (10 mmol) are dissolved in dry acetonitrile and the reaction mixture is heated under reflux for 12 hours. After cooling to r.t. the product can be filtered off and washed with acetonitrile. It can be used for the next reaction step without further purification. A solution of the corresponding salt (1 mmol) and triethylorthoformiate (2 mmol) in 1 ml dry pyridine is heated under reflux for 16 hours in an argon atmosphere. After cooling to room temperature the reaction mixture is purified by column chromatography (SiO₂; CH₂Cl₂:CH₃OH=10:1) yielding the final product as a colorless solid.

### Example 13

### 2-[3-(1,3-Dihydro-1-{2-[2-(2-methoxyethoxy)ethoxy]ethyl}-3,3-dimethyl-2H-indol-2-yliden)-1-propen-1-yl]-1-{2-[2-(2-methoxyethoxy)ethoxy]ethyl}3H-indolium Chloride (BSc 4738)

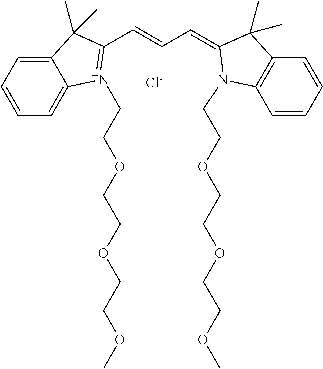 [SMILES: COCCOCCOCCN1/C(=C\C=C\C2=[N+](CCOCCOCCOC)c3ccccc3C2(C)C)C(C)(C)c2ccccc21 ; [Cl-]]

¹H-NMR (500 MHz, DMSO): δ=8.41-8.33 (t, J=13.0 Hz, 1H), 7.65-7.61 (d, J=7.0 Hz, 2H), 7.48-7.44 (d, J=8.0 Hz, 2H), 7.44-7.39 (td, J=8.0 Hz, J=1.0 Hz, 2H), 7.31-7.26 (td, J=8.0 Hz, J=1.0 Hz, 2H), 6.58-6.52 (d, J=13.5 Hz, 2H), 4.38-4.30 (t, J=5.0 Hz, 4H), 3.85-3.78 (t, J=5.0 Hz, 4H), 3.55-3.50 (m, 4H), 3.44-3.40 (m, 4H), 3.39-3.36 (m, 4H), 3.31-3.27 (m, 4H), 3.17-3.14 (s, 6H), 1.72-1.66 (s, 12H) ppm.

¹³C-NMR (500 MHz, DMSO): δ=174.47, 149.74, 142.11, 140.28, 128.34, 125.01, 122.23, 111.91, 103.06, 71.10, 70.23, 69.67, 69.51, 67.11, 57.90, 48.82, 44.12, 27.36 ppm

MS (EI): m/z=622

### Example 14

### 2-[3-[1,3-Dihydro-1-(2-methoxyethyl)-3,3-dimethyl-2H-indol-2-yliden]-1-propen-1-yl3H-indolium Chloride (BSc 4741)

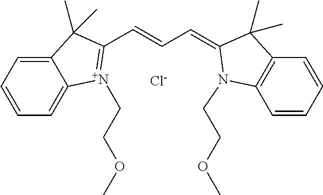 [SMILES: COCCN1/C(=C\C=C\C2=[N+](CCOC)c3ccccc3C2(C)C)C(C)(C)c2ccccc21 ; [Cl-]]

¹H-NMR (500 MHz, DMSO): δ=8.43-8.35 (t, J=13.0 Hz, 1H), 7.65-7.61 (d, J=7.0 Hz, 2H), 7.48-7.40 (m, 4H), 7.32-7.27 (td, J=8.0 Hz, J=1.5 Hz, 2H), 6.55-6.50 (d, J=13.5 Hz, 2H), 4.37-4.31 (t, J=5.5 Hz, 4H), 3.28-3.24 (s, 6H), 1.72-1.65 (s, 12H) ppm.

¹³C-NMR (500 MHz, DMSO): δ=174.51, 149.88, 142.16, 140.29, 128.39, 125.05, 122.26, 111.84, 102.96, 68.77, 58.59, 48.84, 44.09, 27.40 ppm

MS (ESI): m/z=445.2

### Example 15

### 2-[3-(1,3-Dihydro-1,3,3-trimethyl-2H-indol-2-yliden)-1-propen-1-yl]-1,3,3-trimethyl-3H-indolium Iodide (BSc 4704)

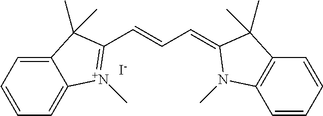 [SMILES: CN1/C(=C\C=C\C2=[N+](C)c3ccccc3C2(C)C)C(C)(C)c2ccccc21 ; [I-]]

¹H-NMR (500 MHz, DMSO): δ=8.37-8.30 (t, J=13.5 Hz, 1H), 7.64-7.60 (d, J=7.0 Hz, 2H), 7.46-7.42 (m, 4H), 7.32-7.27 (m, 2H), 6.46-6.41 (d, J=13.5 Hz, 2H), 3.66-3.62 (s, 6H), 1.72-1.65 (s, 12H) ppm.

¹³C-NMR (500 MHz, DMSO): δ=174.44, 149.64, 142.72, 140.59, 128.67, 125.28, 122.48, 111.52, 102.72, 48.92, 31.44, 27.36 ppm

MS (ESI): m/z=357.1

### Example 16

### 5-Nitro-2-[3-(5-nitro-1,3-dihydro-1,3,3-trimethyl-2H-indol-2-yliden)-1-propen-1-yl]-1,3,3-trimethyl-3H-indolium Iodide (BSc 4705 (JG 268))

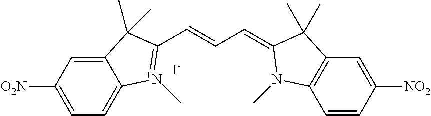 [SMILES: CN1/C(=C\C=C\C2=[N+](C)c3ccc([N+](=O)[O-])cc3C2(C)C)C(C)(C)c2cc([N+](=O)[O-])ccc21 ; [I-]]

¹H-NMR (500 MHz, DMSO): δ=8.61-8.59 (d, J=2.5 Hz, 2H), 8.47-8.40 (t, J=13.5 Hz, 1H), 8.40-8.36 (dd, J=9.0 Hz, J=2.5 Hz, 2H), 7.72-7.69 (d, J=9.0 Hz, 2H), 6.76-6.71 (d, J=8.5 Hz, 2H), 3.75-3.72 (s, 6H), 1.78-1.74 (s, 12H) ppm.

¹³C-NMR (500 MHz, DMSO): δ=178.47, 151.52, 147.80, 144.54, 141.86, 125.41, 118.25, 112.08, 105.73, 54.83, 48.96, 32.17, 26.82 ppm

MS (EI): m/z=446

### Example 17

### 2-[3-(1,3-Dihydro-3,3-dimethyl-1-octyl-2H-indol-2-yliden)-1-propen-1-yl]-3,3-dimethyl-1-octyl-indolium Bromide (BSc4737 (JG 279))

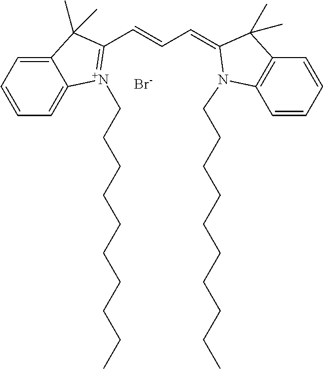 [SMILES: CCCCCCCCCCN1/C(=C\C=C\C2=[N+](CCCCCCCCCC)c3ccccc3C2(C)C)C(C)(C)c2ccccc21 ; [Br-]]

¹H-NMR (500 MHz, DMSO): δ=8.39-8.31 (t, J=13.5 Hz, 1H), 7.67-7.63 (d, J=7.5 Hz, 2H), 7.49-7.45 (d, J=7.5 Hz, 2H), 7.45-7.40 (td, J=8.0 Hz, J=1.0 Hz, 2H), 7.31-7.26 (td, J=8.0 Hz, J=1.0 Hz, 2H), 6.68-6.62 (d, J=13.5 Hz, 2H), 4.19-4.11 (t, J=7.5 Hz, 4H), 1.76-1.68 (m, 4H), 1.71-1.64 (s, 12H), 1.44-1.35 (m, 4H), 1.35-1.26 (m, 4H), 1.26-1.14 (m, 20H), 0.83-0.78 (t, J=7.0 Hz, 6H), ppm.

¹³C-NMR (500 MHz, DMSO): δ=173.62, 149.71, 141.74, 140.49, 128.52, 125.06, 122.41, 111.46, 102.66, 48.74, 43.69, 31.14, 28.76, 28.74, 28.66, 28.54, 27.34, 26.94, 25.91, 21.94, 13.78 ppm

MS (ESI): m/z=609.4

### Example 18

### 2-[3-(1,3-Dihydro-3,3-dimethyl-1-butyl-2H-indol-2-yliden)-1-propen-1-yl]-3,3-dimethyl-1-butyl-indolium Iodide (BSc4739)

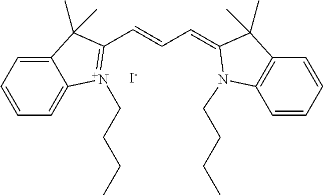 [SMILES: CCCCN1/C(=C\C=C\C2=[N+](CCCC)c3ccccc3C2(C)C)C(C)(C)c2ccccc21 ; [I-]]

¹H-NMR (500 MHz, DMSO): δ=8.40-8.32 (t, J=13.5 Hz, 1H), 7.66-7.62 (d, J=7.0 Hz, 2H), 7.49-7.41 (m, 4H), 7.32-7.27 (td, J=7.5 Hz, J=1.5 Hz, 2H), 6.60-6.55 (d, J=13.5 Hz, 2H), 4.17-4.11 (t, J=7.5 Hz, 4H), 1.77-1.71 (m, 4H), 1.71-1.66 (s, 12H), 1.48-1.40 (m, 4H), 0.97-0.92 (t, J=7.5 Hz, 6H), ppm.

¹³C-NMR (500 MHz, DMSO): δ=173.74, 149.80, 141.78, 140.54, 128.54, 125.09, 122.41, 111.47, 102.47, 48.79, 43.61, 29.12, 17.36, 19.45, 13.70 ppm

MS (ESI): m/z=441.3

### Example 19

### 5-Bromo-2-[3-(5-bromo-1,3-dihydro-1,3,3-trimethyl-2H-indol-2-yliden)-1-propen-1-yl]-1,3,3-trimethyl-3H-indolium Iodide (BSc 4742)

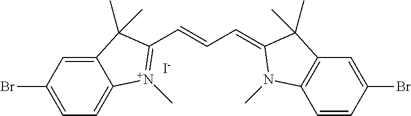 [SMILES: CN1/C(=C\C=C\C2=[N+](C)c3ccc(Br)cc3C2(C)C)C(C)(C)c2cc(Br)ccc21 ; [I-]]

¹H-NMR (500 MHz, DMSO): δ=8.34-8.26 (t, J=13.5 Hz, 1H), 7.94-7.91 (d, J=2.0 Hz, 2H), 7.66-7.62 (dd, J=8.5 Hz, J=2.0 Hz, 2H), 7.45-7.40 (d, J=8.5 Hz, 2H), 6.48-6.42 (d, J=13.5 Hz, 2H), 3.65-3.60 (s, 6H), 1.71-1.65 (s, 12H) ppm.

¹³C-NMR (500 MHz, DMSO): δ=174.08, 149.58, 142.71, 141.93, 131.17, 125.52, 117.43, 113.25, 103.09, 48.93, 31.49, 26.94, 26.22 ppm

MS (ESI): m/z=515.0

### Example 20

### 3-Methyl-2-[3-(3-methyl-2(3H)-benzothiazolyliden)-1-propenyl]-benzothiazolium Iodide BSc4706 ( )

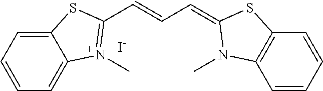

¹H-NMR (500 MHz, DMSO): δ=7.99-7.93 (d, J=8.0 Hz, 2H), 7.74-7.66 (m, 3H), 7.56-7.50 (t, J=8.0 Hz, 2H), 7.40-7.34 (t, J=8.0 Hz, 2H), 6.56-6.49 (d, J=13.0 Hz, 2H), 3.84-3.76 (s, 6H) ppm.

¹³C-NMR (500 MHz, DMSO): δ=164.77, 145.93, 141.66, 127.85, 124.98, 124.71, 122.81, 113.43, 98.81, 33.41 ppm

MS (ESI): m/z=337

### Example 21

### Staining Protocol for the Compounds of this Invention

Tissue samples were fixed in 10% buffered formalin solution and embedded in paraffin. Slices of 4 μm thickness were prepared using a microtome and mounted on a slide in a water bath. Deparaffinization was carried out according to the following steps:

Xylene 15 min, 100% ethanol 10 min, 96% ethanol 10 min, 70% ethanol 10 min and storage in water until staining.

Staining with dyes of this invention was subsequently performed according to the following procedure steps:

| Procedure step | Reagent | Time | | Comment |
|---|---|---|---|---|
| 1. | Acidic hematoxylin | 10 | min | Cell nucleus |
| | | | | staining |
| 2. | Blueing-up in tap | 5 | min | — |
| | water | | | |
| 3. | Staining solution | 5 | min | See remark |
| 4. | EtOH/MeOH rinse | — | | with 3-6 ml |
| 5. | Rinse with tap water | 3-5 | min | — |
| 6. | 1% acetic acid | 10-20 | min | Differentiation |
| 7. | Rinse with tap water | 5 | min | — |

| | | | |
|---|---|---|---|
| 8. | Cover | — | with glycerin- |
| | | | buffer-mixture |

Remark concerning step 3:

Dyes of the present invention were applied in 0.01-1% ethanolic or methanolic solution dropwise onto the tissue section (50-200 μL) and incubated in a moist, EtOH/MeOH saturated and light-protected chamber for 10 min.

In the case of poorly soluble substances, up to 10% DMSO were added and, if required, filtered through a syringe filter (0.45 μm pore size).

### Example 22

### Microscopy of Compounds of the Present Invention

Samples were stained with dyes of the present invention as described in example 21 and subsequently investigated using a Zeiss Axioskop, ABO 100 Hg fluorescent lamp, camera: Leica DFC300FX. Depending on the respective dye used, either a FITC filter or a DAPI filter was utilized. Results are presented in FIGS. 1-21 which demonstrate the binding and visualization of the dyes of the present invention.

### Example 23

### Radioligand Competition Assay

The affinity of inventive compounds was investigated using a radioligand competition assay. For this purpose, Aβ-(1-42) peptide in a concentration of 10 mg/ml in PBS with 0.1% BSA was incubated together with [125I]IMPY 0.1 nM and different ligand concentrations for 3 h at 20° C., followed by filtration through a Whatman GF/B filter.

### FIGURE DESCRIPTIONS

FIG. 1: Staining with BSc4258, human olfactory epithelium, patient AD+; Tau-aggregate, FITC-Filter, Zeiss Axioskop, ABO 100 Hg fluorescent lamp, camera: Leica DFC300FX

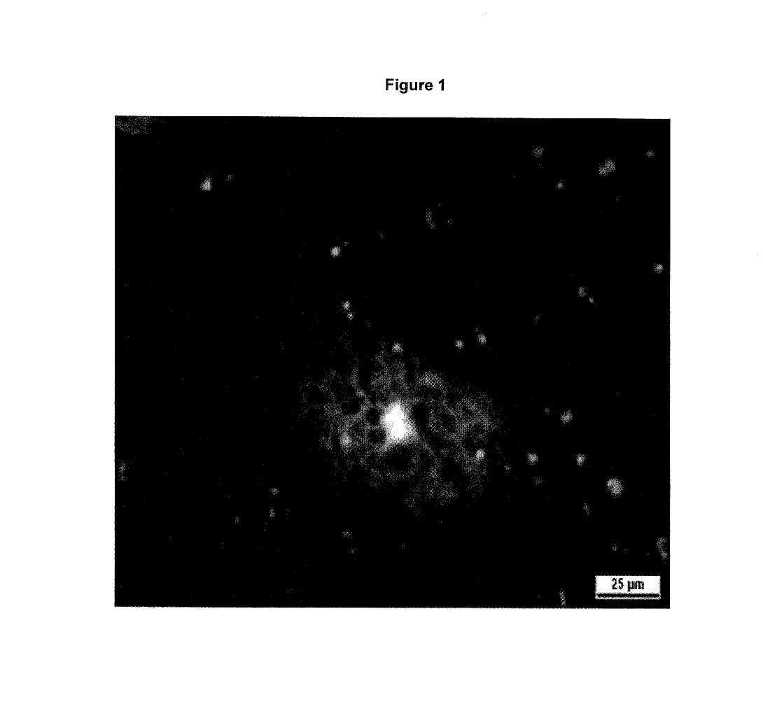

FIG. 2: Staining with BSc4258, human brain tissue, patient AD+; amyloid plaque), FITC-Filter, Zeiss Axioskop, ABO 100 Hg fluorescent lamp, camera: Leica DFC300FX

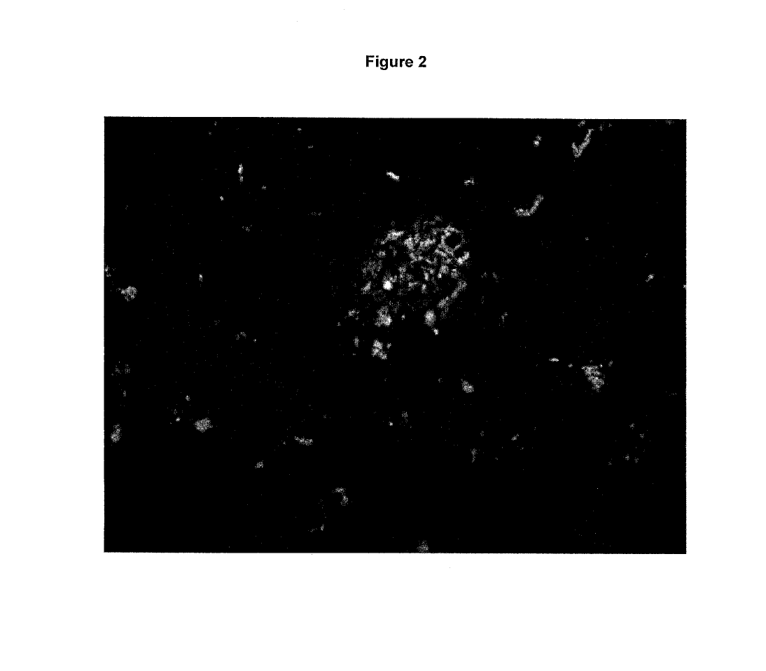

FIG. 3: Staining with BSc4090, hippocampus, male 89 yo, AD+, Aβ plaque, FITC filter, Zeiss Axioskop, ABO 100 Hg fluorescent lamp, camera: Leica DFC300FX

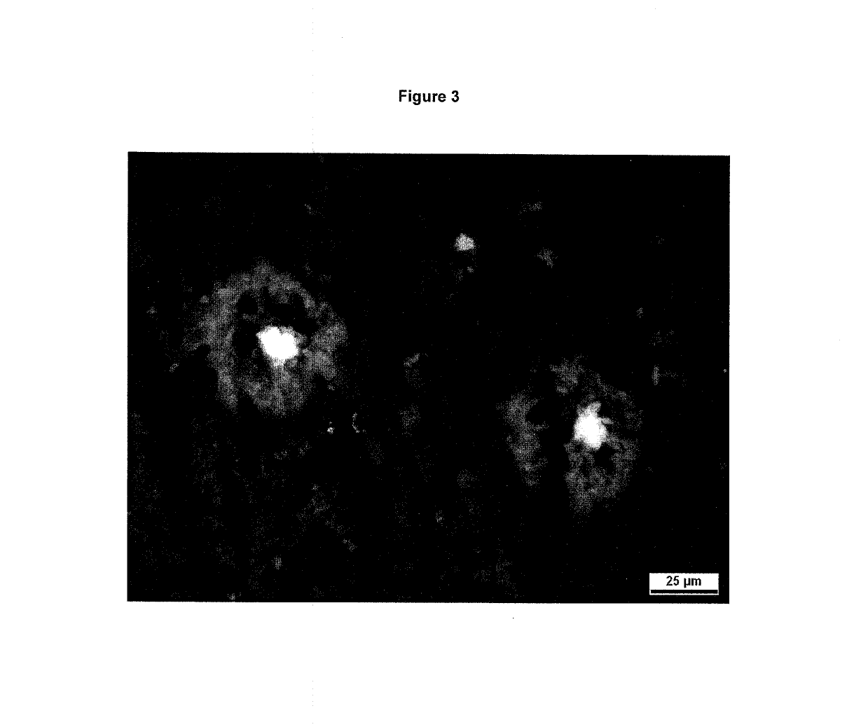

FIG. 4: Staining with BSc4090, hippocampus, male 89 yo, AD+, Aβ plaque, DAPI filter, Zeiss Axioskop, ABO 100 Hg fluorescent lamp, camera: Leica DFC300FX

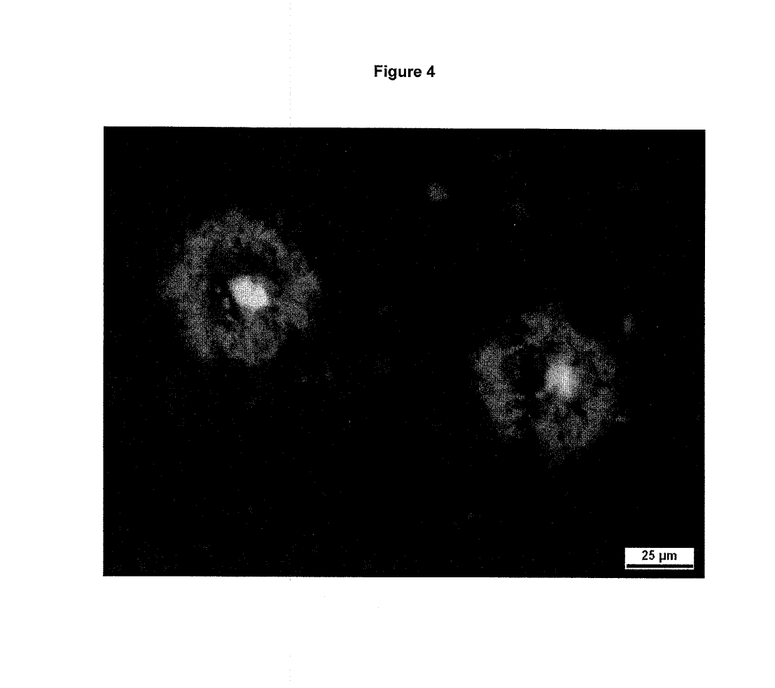

FIG. 5: Staining with BSc4097, hippocampus, male 89 yo, AD+, Aβ plaque, DAPI filter, Zeiss Axioskop, ABO 100 Hg-fluorescent lamp, camera: Leica DFC300FX

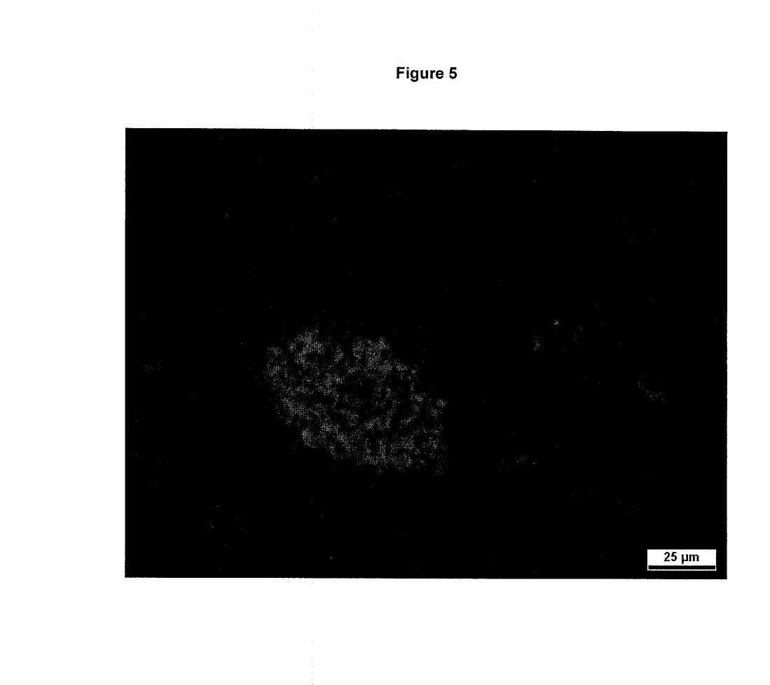

FIG. 6: Staining with BSc4097, hippocampus, male 89 yo, AD+, Aβ plaque (overview), DAPI filter, Zeiss Axioskop, ABO 100 Hg-fluorescent lamp, camera: Leica DFC300FX

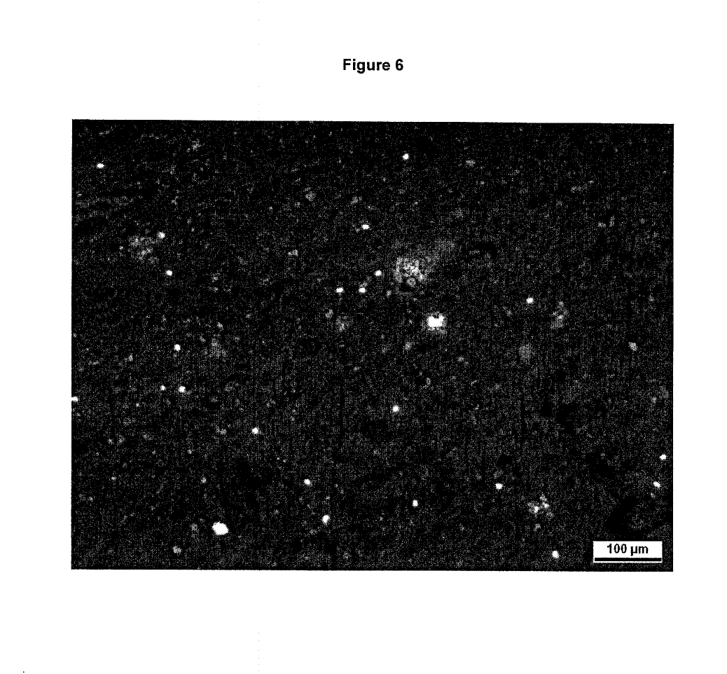

FIG. 7: Staining with BSc4327, hippocampus, male 82 yo, AD+, Aβ in angiopathy, FITC filter, Zeiss Axioskop, ABO 100 Hg-fluorescent lamp, camera: Leica DFC300FX

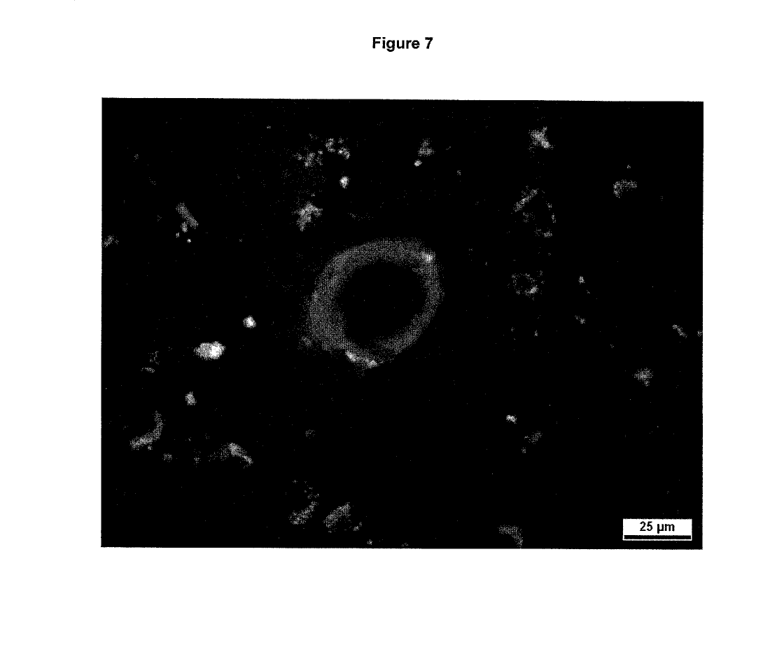

FIG. 8: Staining with BSc4328, hippocampus, male 82 yo, AD+, Aβ plaque, DAPI filter, Zeiss Axioskop, ABO 100 Hg fluorescent lamp, camera: Leica DFC300FX

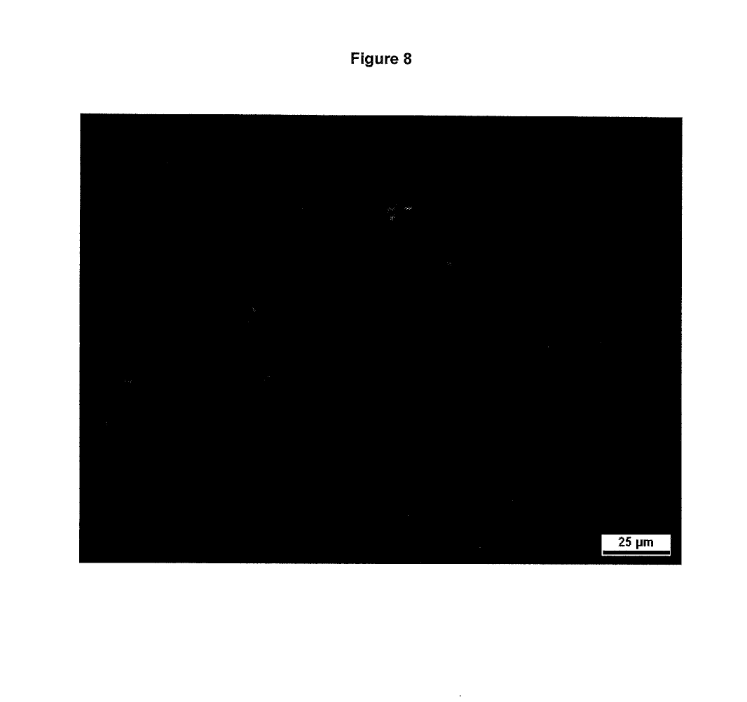

FIG. 9: Staining with BSc4352, hippocampus, male 82 yo, A+, Aβ plaque, DAPI filter, Zeiss Axioskop, ABO 100 Hg fluorescent lamp, camera: Leica DFC300FX

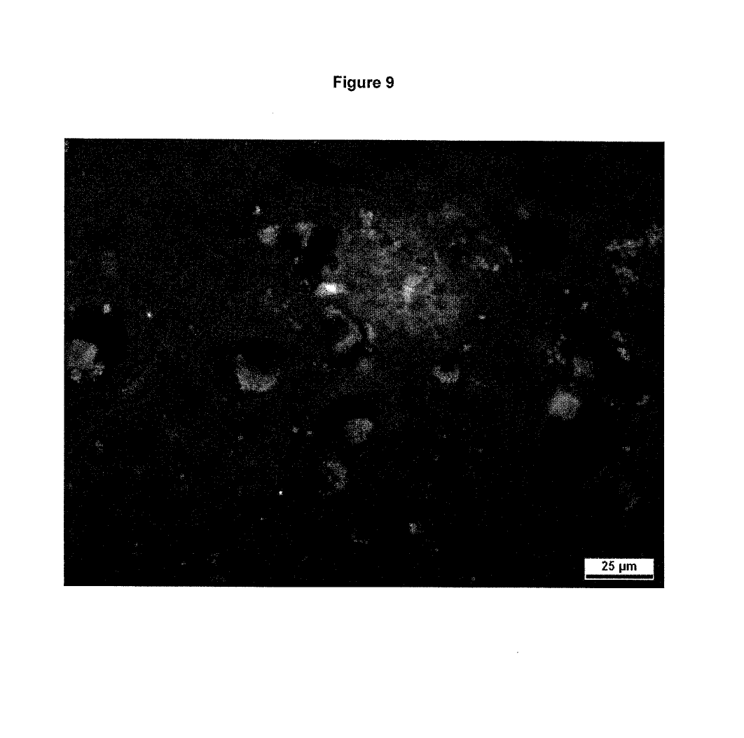

FIG. 10: Staining with BSc4352, hippocampus, male 82 yo, AD+, Aβ plaque, DAPI filter, Zeiss Axioskop, ABO 100 Hg fluorescent lamp, camera: Leica DFC300FX

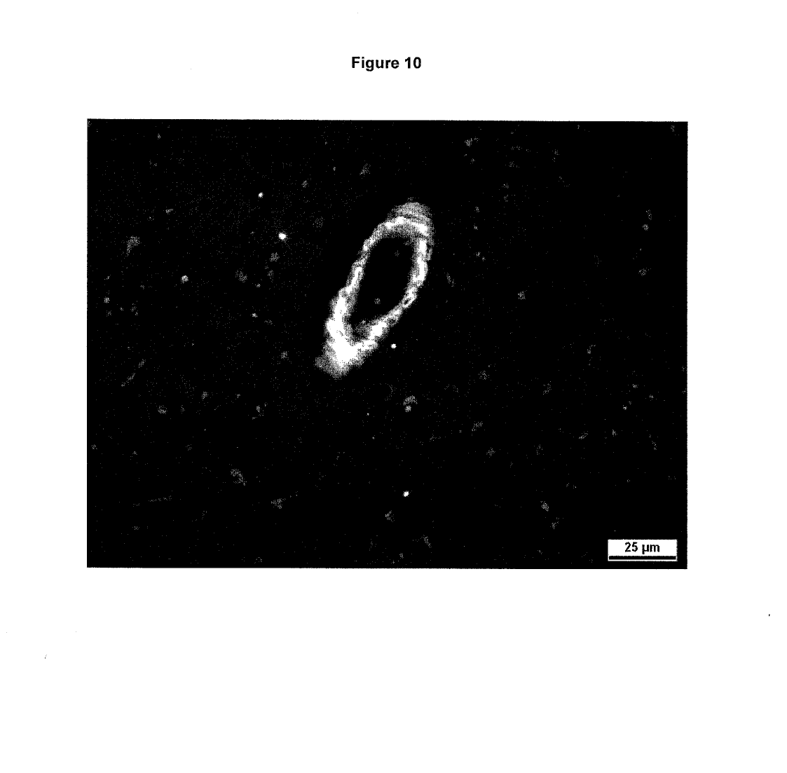

FIG. 11: Staining with BSc4353, hippocampus, male 82 yo, AD+, Aβ in angiopathy, FITC filter, Zeiss Axioskop, ABO 100 Hg fluorescent lamp, camera: Leica DFC300FX

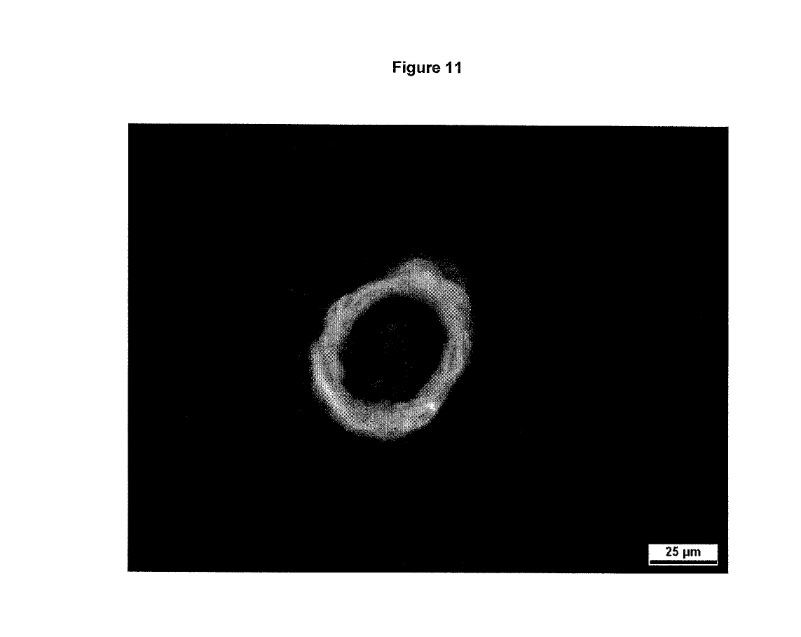

FIG. 12: Staining with BSc4353, hippocampus, male 89 yo, AD+, Aβ plaque, FITC filter, Zeiss Axioskop, ABO 100 Hg fluorescent lamp, camera: Leica DFC300FX

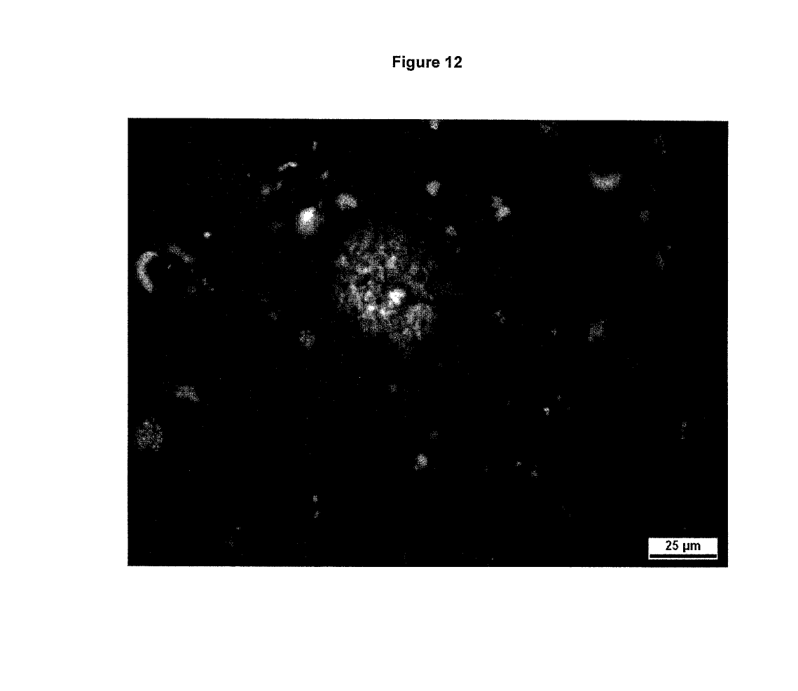

FIG. 13: Staining with BSc4354, hippocampus, male 82 yo, AD+, Aβ in angiopathy, FITC filter, Zeiss Axioskop, ABO 100 Hg fluorescent lamp, camera: Leica DFC300FX

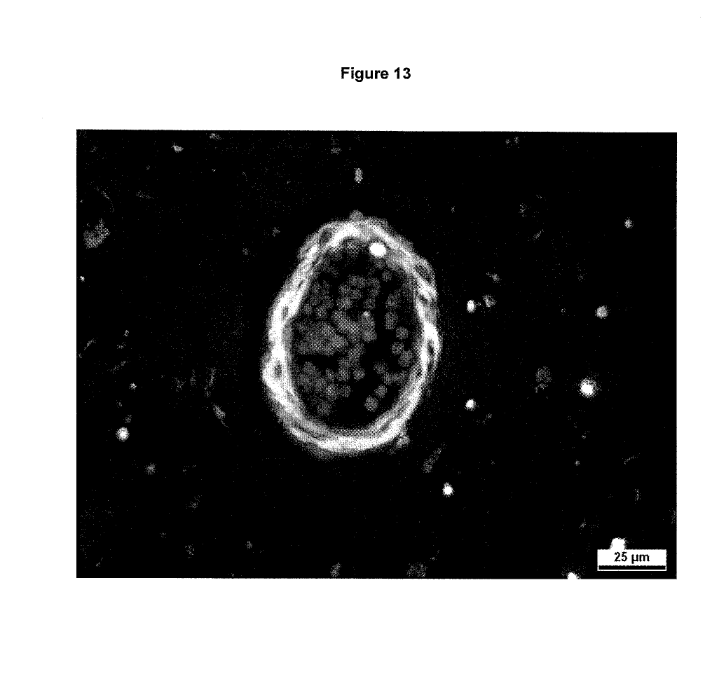

FIG. 14: Staining with BSc4354, hippocampus, male 89 yo, AD+, Aβ plaque, FITC filter, Zeiss Axioskop, ABO 100 Hg fluorescent lamp, camera: Leica DFC300FX

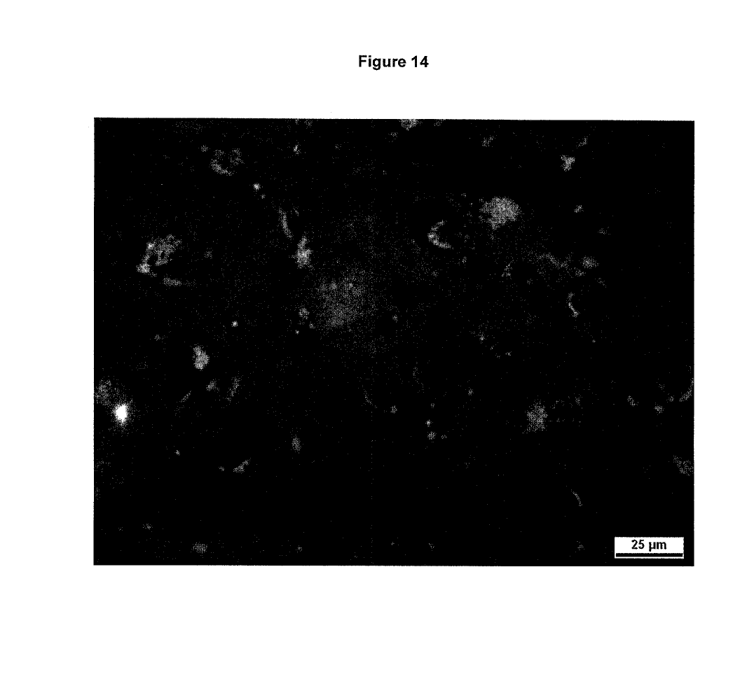

FIG. 15: Staining with BSc4342 hippocampus, male 89 yo, AD+, Aβ Plaque, DAPI-Filter, Zeiss Axioskop, ABO 100 Hg-fluorescent lamp, Camera: Leica DFC300FX

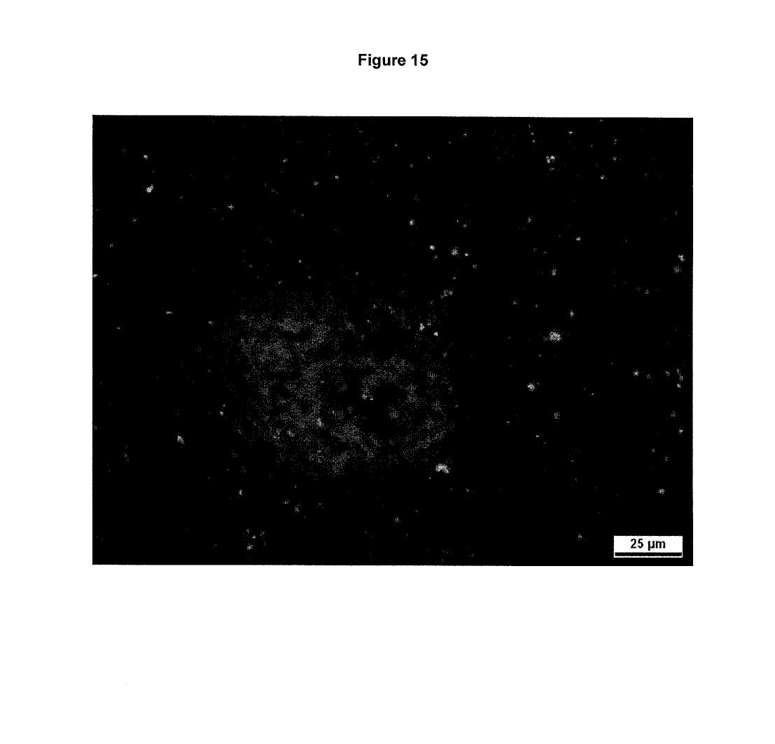

FIG. 16: Staining with BSc4342, hippocampus, male 89 yo, AD+, Aβ plaque, DAPI filter, Zeiss Axioskop, ABO 100 Hg-fluorescent lamp, Camera: Leica DFC300FX

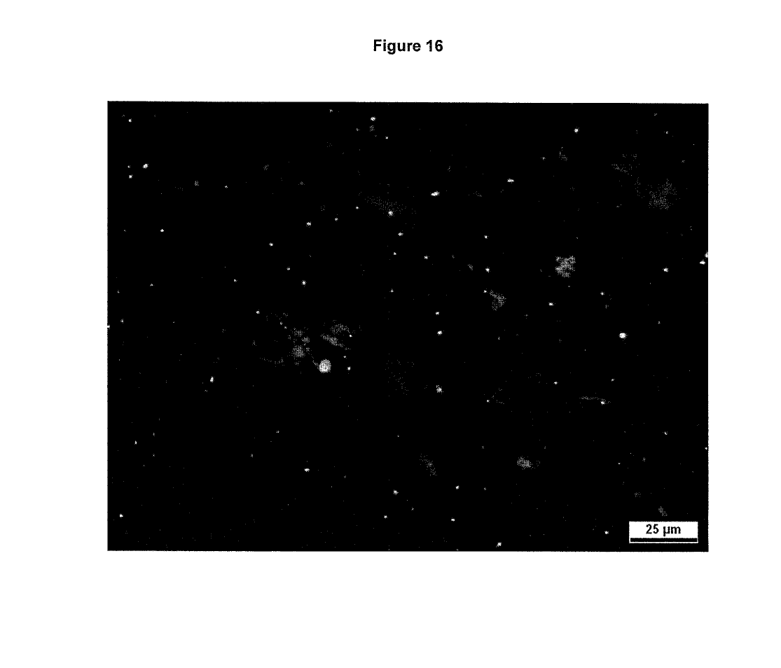

FIG. 17: Staining with BSc4337, hippocampus, male 89 yo, AD+, Aβ plaque, DAPI filter, Zeiss Axioskop, ABO 100 Hg-fluorescent lamp, Camera: Leica DFC300FX

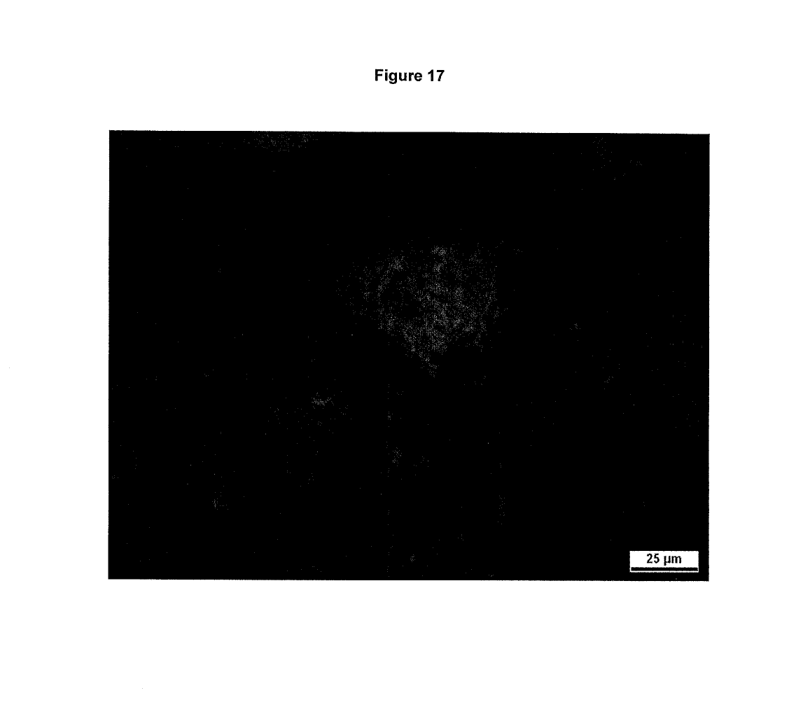

FIG. 18: Staining with BSc4337, hippocampus, male 89 yo, AD+, Aβ plaque, DAPI filter, Zeiss Axioskop, ABO 100 Hg-fluorescent lamp, Camera: Leica DFC300FX

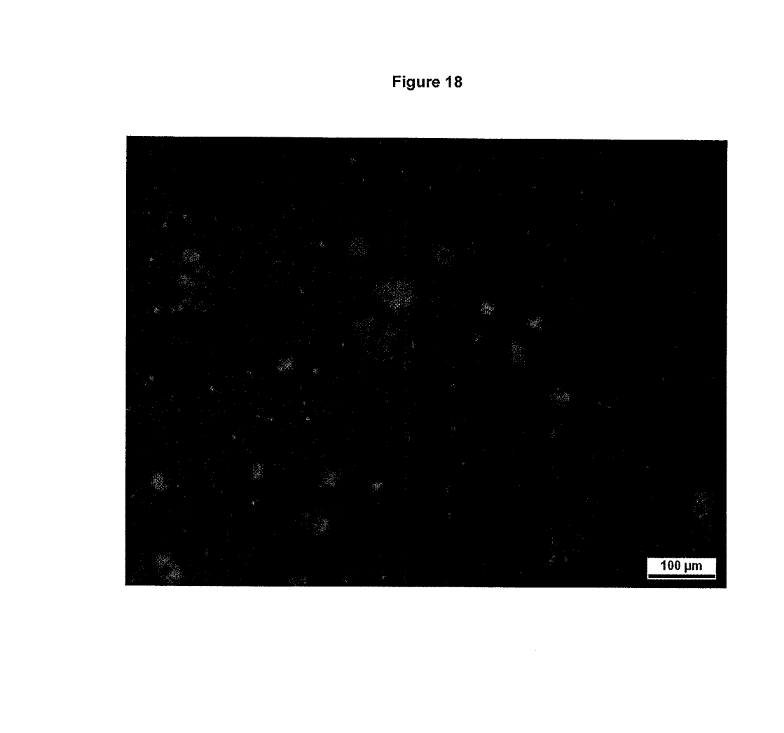

FIG. 19: Staining with BSc4007, hippocampus, male 89 yo, AD, Tau fibril, DAPI filter; Zeiss Axioskop, ABO 100 Hg fluorescent lamp, camera: Leica DFC300FX)

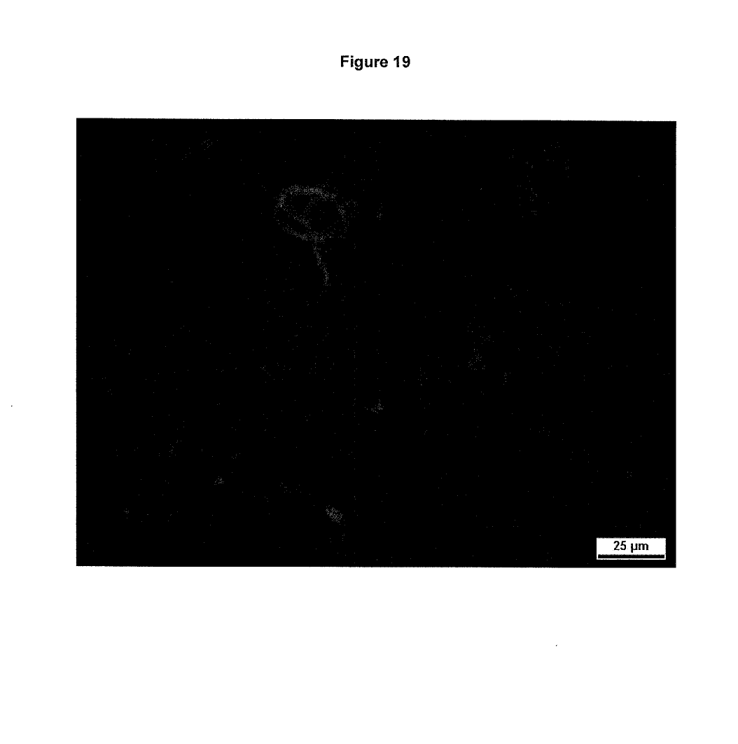

FIG. 20: Staining with BSc4138, hippocampus, male 89 yo, AD, Tau fibril, DAPI filter; Zeiss Axioskop, ABO 100 Hg fluorescent lamp, camera: Leica DFC300FX)

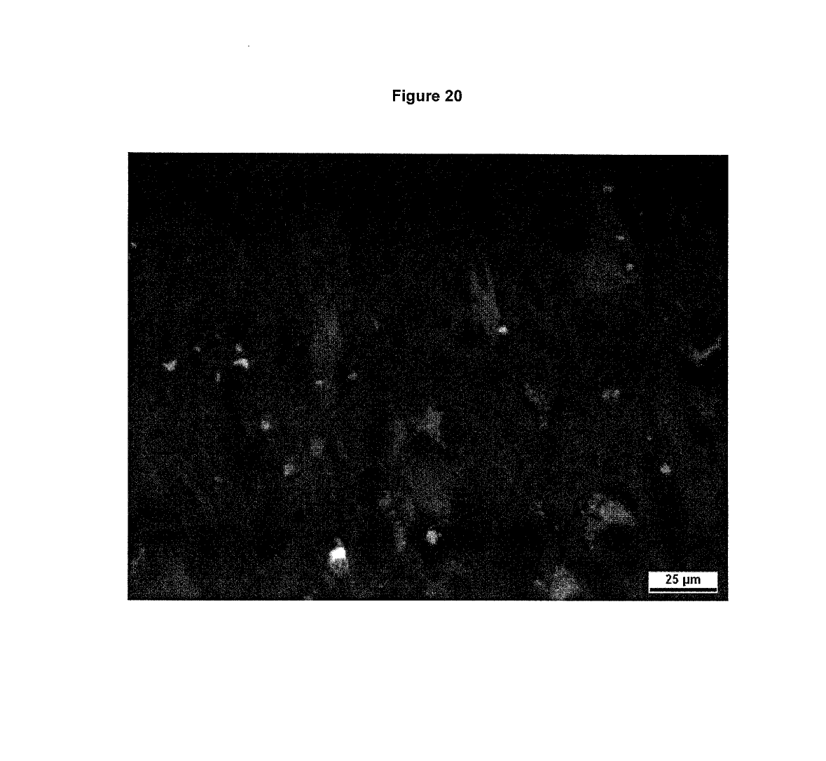

FIG. 21: Staining with BSc4138, hippocampus, male 89 yo, AD, Aβ plaque, DAP filter; Zeiss Axioskop, ABO 100 Hg fluorescent lamp, camera: Leica DFC300FX)

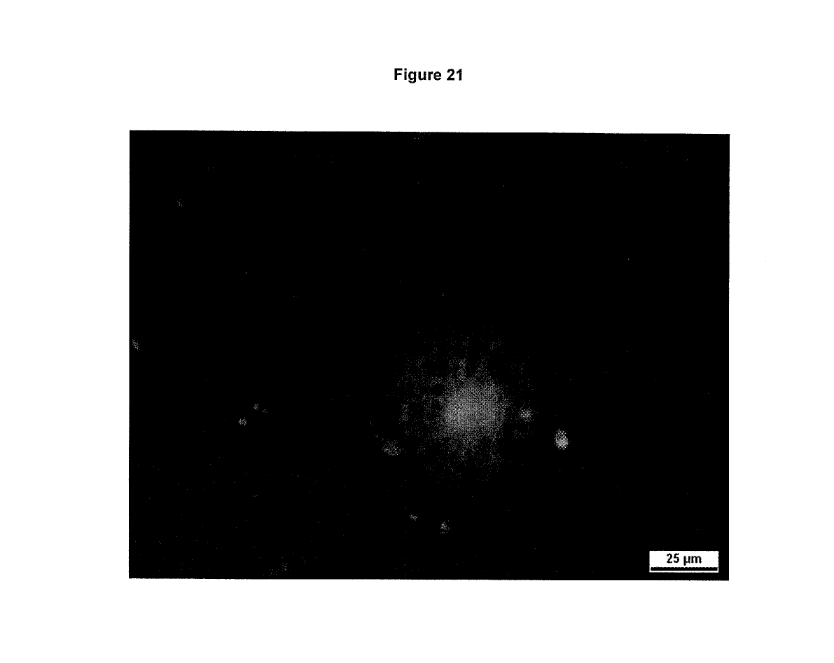

1. A method of using a compound for the diagnosis of neurodegenerative disorders comprising administering to a patient in need thereof the compound, wherein said compound has at least three of the following properties a)-f):
a) a >10fold extinction increase upon binding to the Aβ protein, α-synuclein or to Tau-PHF aggregates as compared to the free compound,
b) a Stokes shift of >20 nm,
c) an extinction coefficient of ε>10 000 L·mol⁻¹·cm⁻¹,
d) EC50<300 nM
e) a log P value between 1 and 2.8,
f) a topological polar surface area (TPSA)<70 Å².

2. The method according to claim 1, wherein a compound of the arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones and/or diaryl ureas is used for the diagnosis of neurodegenerative disorders.

3. The method according to claim 1, wherein the neurodegenerative disease is selected from the group of tauopathies.

4. The method according to claim 1, wherein the neurodegenerative disease is selected from the group comprising or consisting of Alzheimer's disease, corticobasal degeneration, argyrophilic grain disease, Pick's disease, FTDP-17 or progressive supranuclear palsy.

5. The method according to claim 1, wherein the compound specifically binds to Aβ protein, α-synuclein and/or to Tau-PHF aggregates.

6. The method according to claim 1, wherein the compound and a binding thereof to Aβ protein, α-synuclein or to Tau-PHF aggregates can be detected using fiber optics or fluorescence spectroscopy.

7. The method according to claim 1, wherein the arylaminothiazoles have the following general structure: 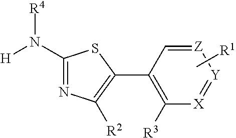
wherein
X, Y, Z are, independently of one another, carbon or nitrogen and
R¹, R², R³, R⁴ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph;
as well as salts, enantiomers, enantiomeric mixtures, diastereomers, diastereomeric mixtures, tautomers, hydrates, solvates and racemates of the aforementioned compounds.

8. The method according to claim 1, whereby said 4,6-divinylpyrimidines have the following general structure: 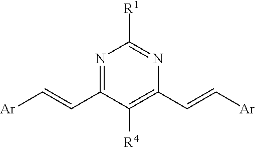
wherein
Ar represents one of the following aromatic groups: 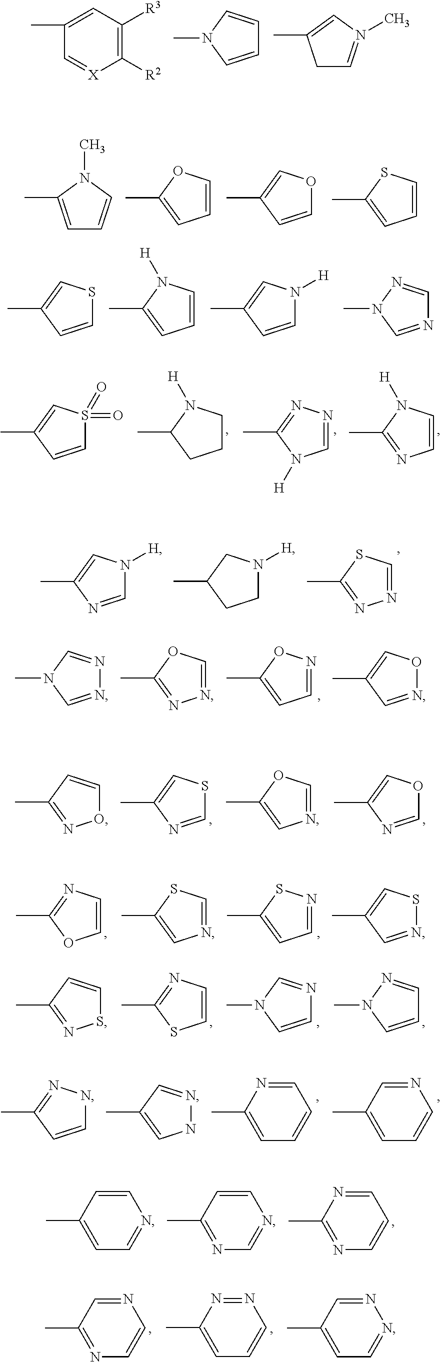 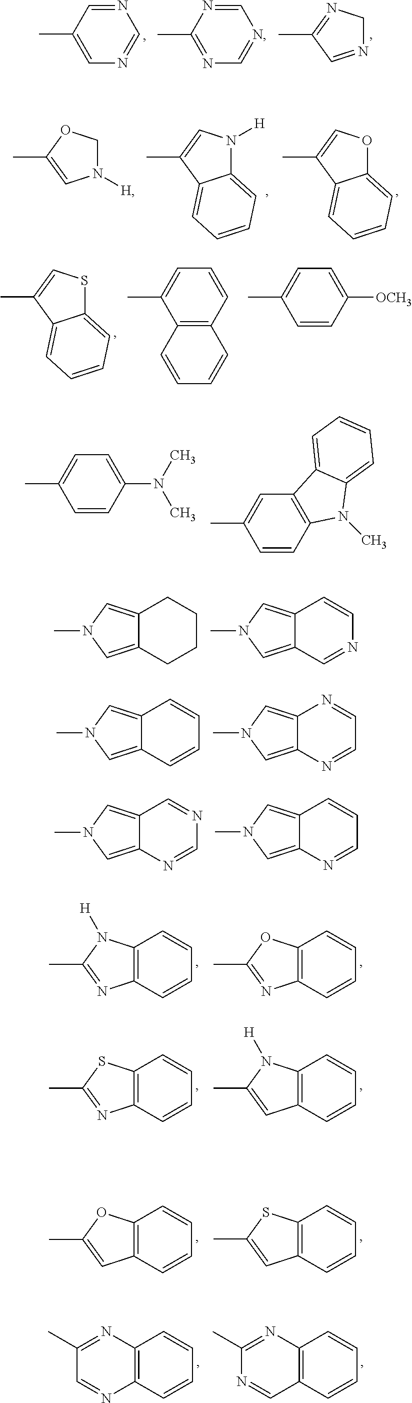 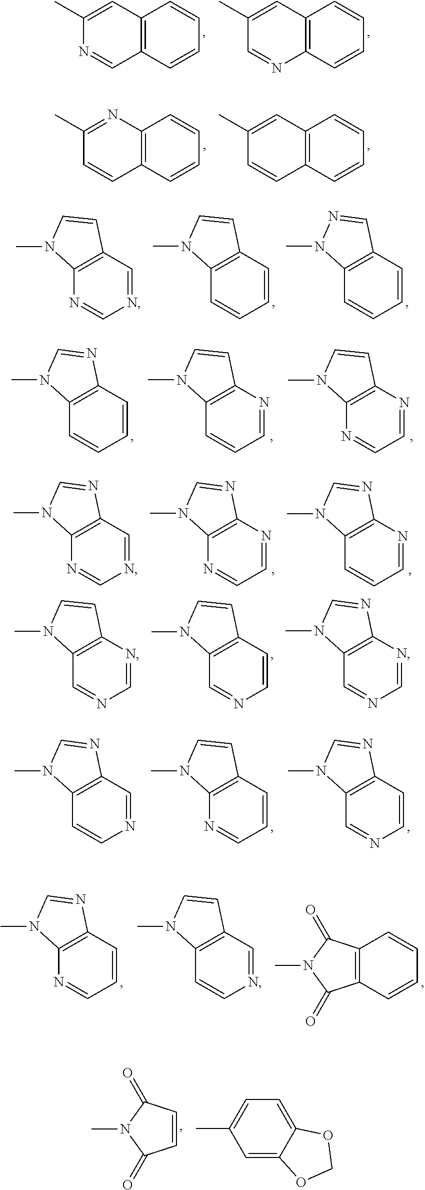
wherein
X is carbon or nitrogen and
R¹, R², R³ and R⁴ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), NH(C₂-C₆-alkynyl), N(C₁-C₆-alkyl)₂, N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph;
as well as salts, enantiomers, enantiomeric mixtures, diastereomers, diastereomeric mixtures, tautomers, hydrates, solvates and racemates of the aforementioned compounds.

9. The method according to claim 1, wherein the 2,5-divinylpyrazines have the following general structure: 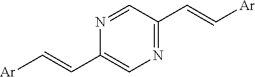
wherein
Ar represents one of the following cyclic, heterocyclic, aromatic or heteroaromatic groups: 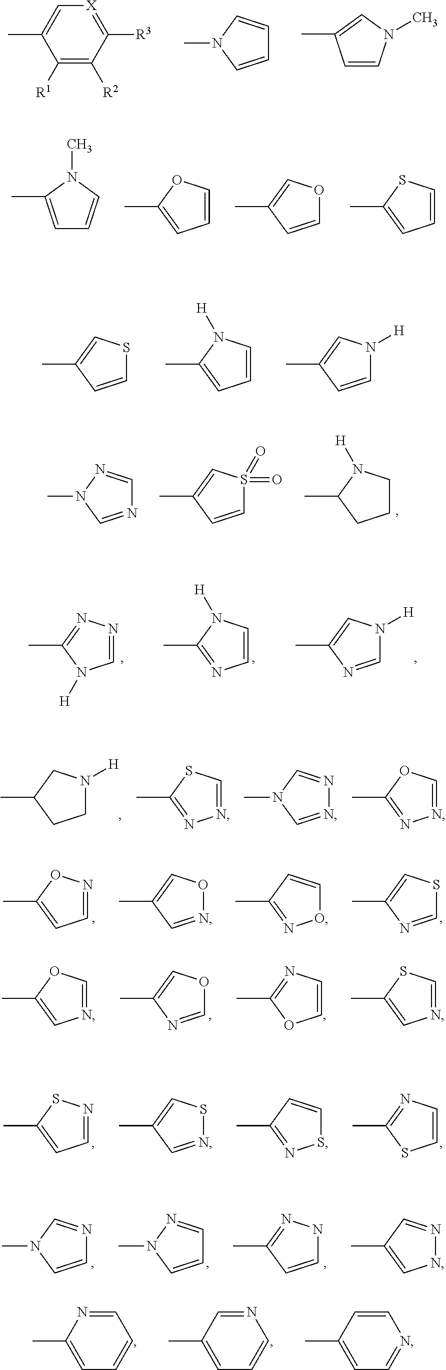 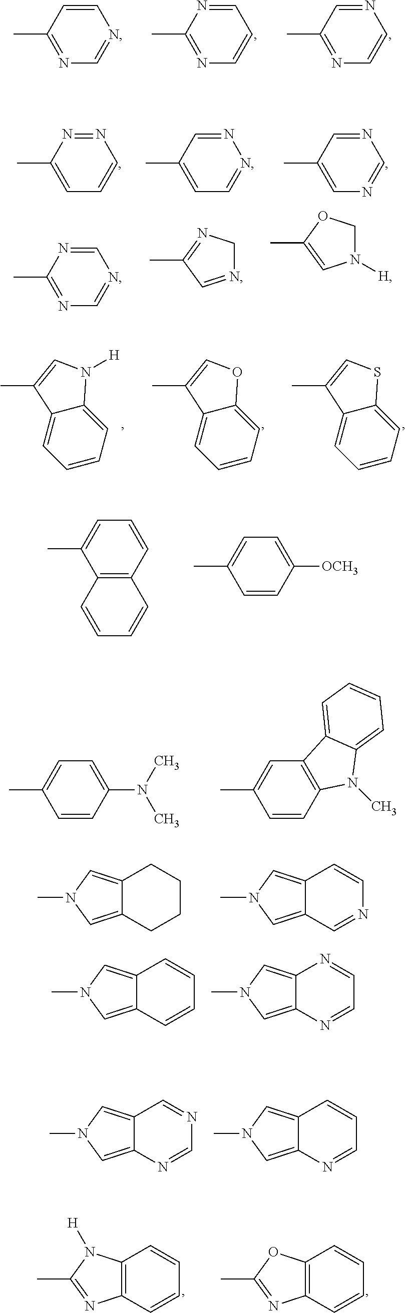 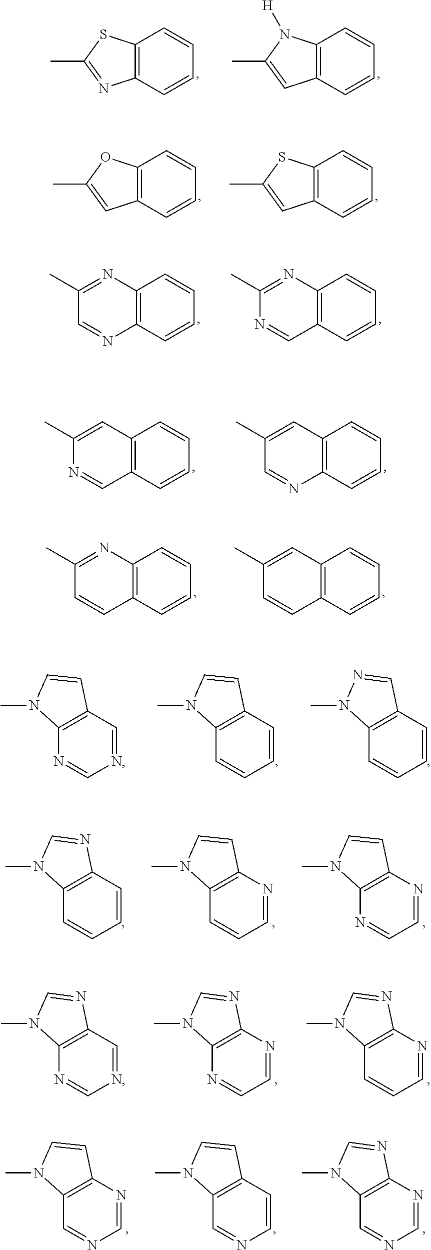 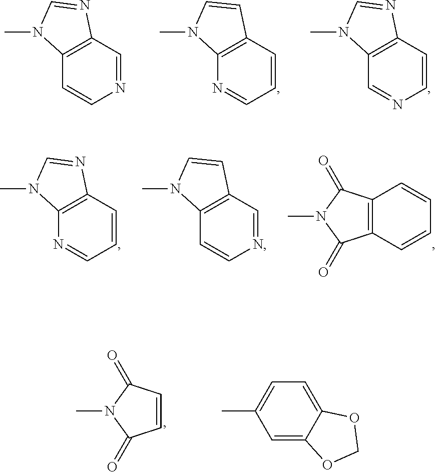
wherein
X is carbon or nitrogen and
R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C—C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph;
as well as salts, enantiomers, enantiomeric mixtures, diastereomers, diastereomeric mixtures, tautomers, hydrates, solvates and racemates of the aforementioned compounds.

10. The method according to claim 1, wherein the [4-(1,3-benzothiazol-2-yl)phenyl]hydrazones have the following general structure: 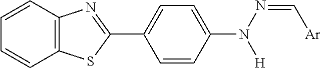
wherein
Ar represents one of the following cyclic, heterocyclic, aromatic or heteroaromatic groups: 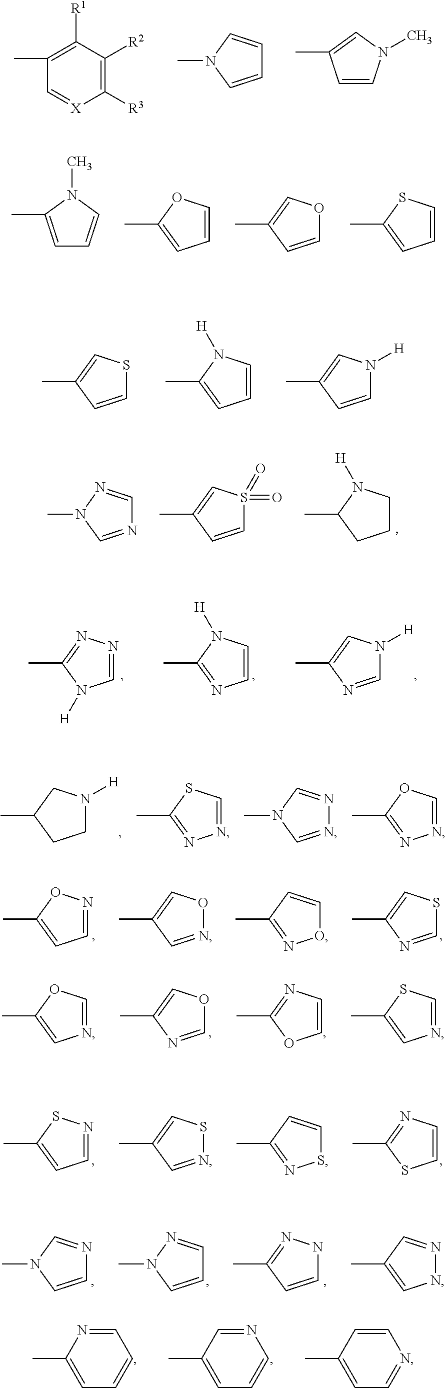 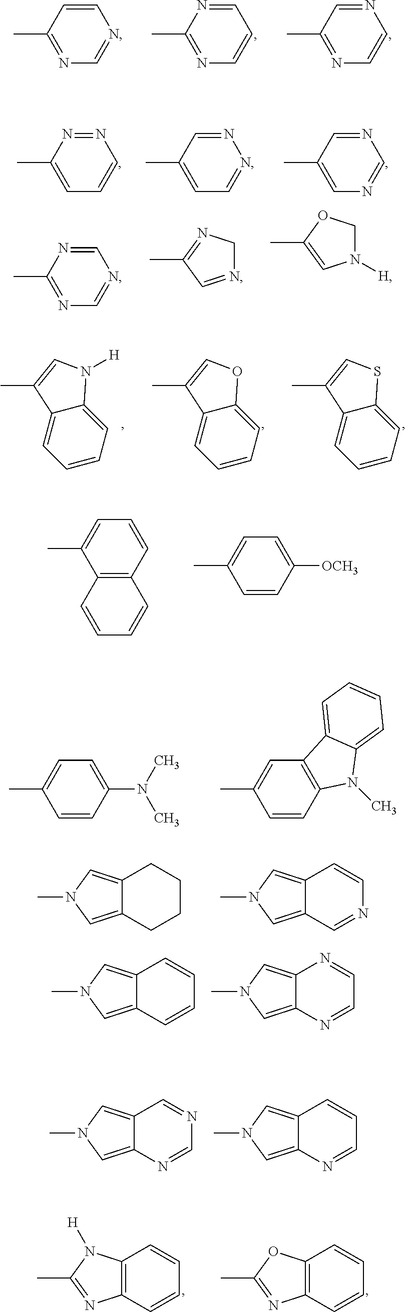 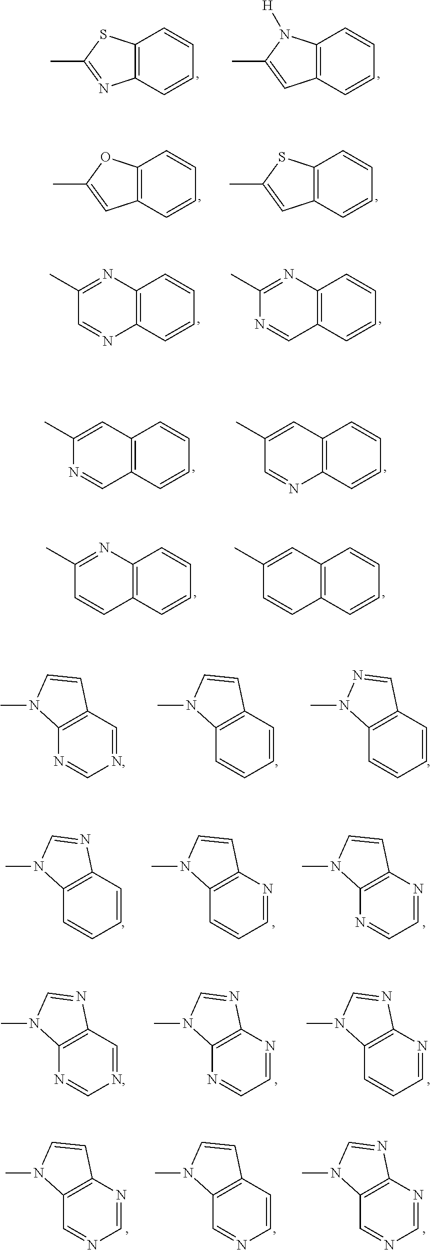 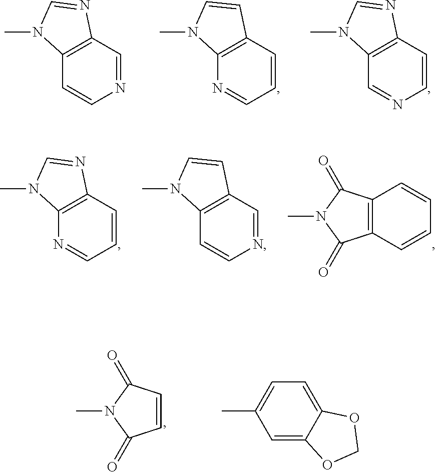
wherein
X is carbon or nitrogen and
R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph;
as well as salts, enantiomers, enantiomeric mixtures, diastereomers, diastereomeric mixtures, tautomers, hydrates, solvates and racemates of the aforementioned compounds.

11. The method according to claim 1, wherein the 3,6-divinylpyridazines have the following general structure: 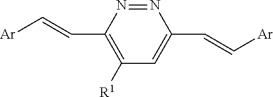
wherein
Ar represents one of the following cyclic, heterocyclic, aromatic or heteroaromatic groups: 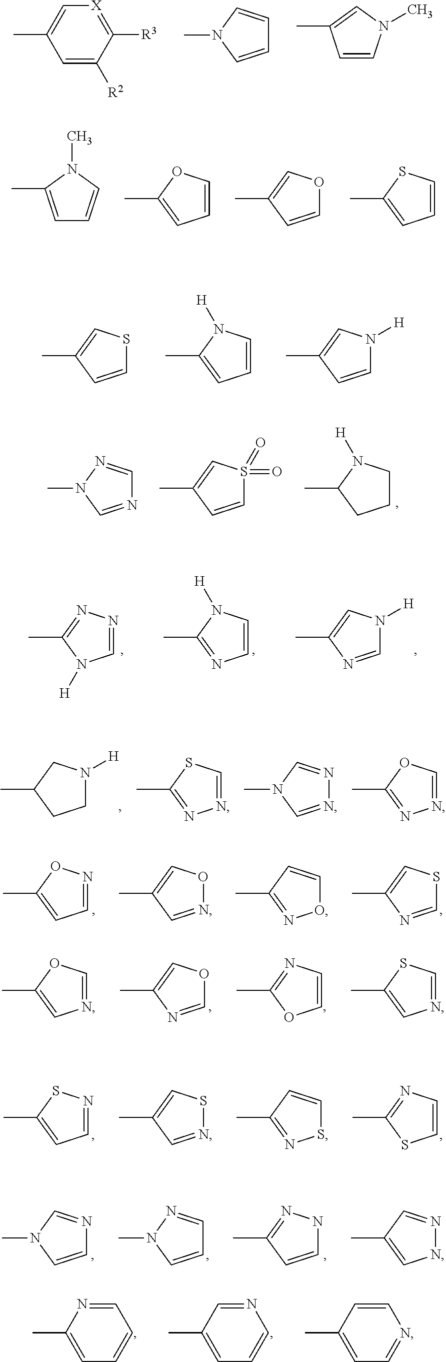 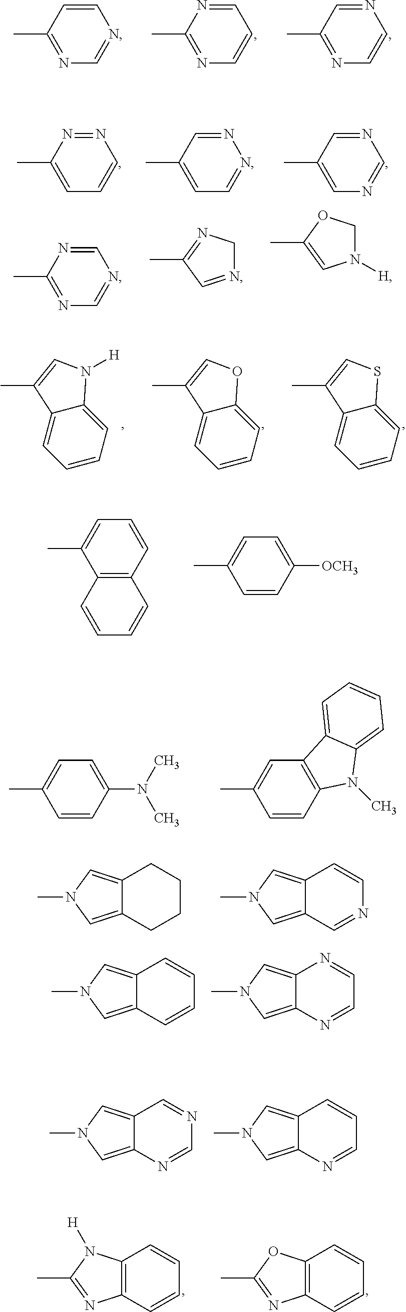 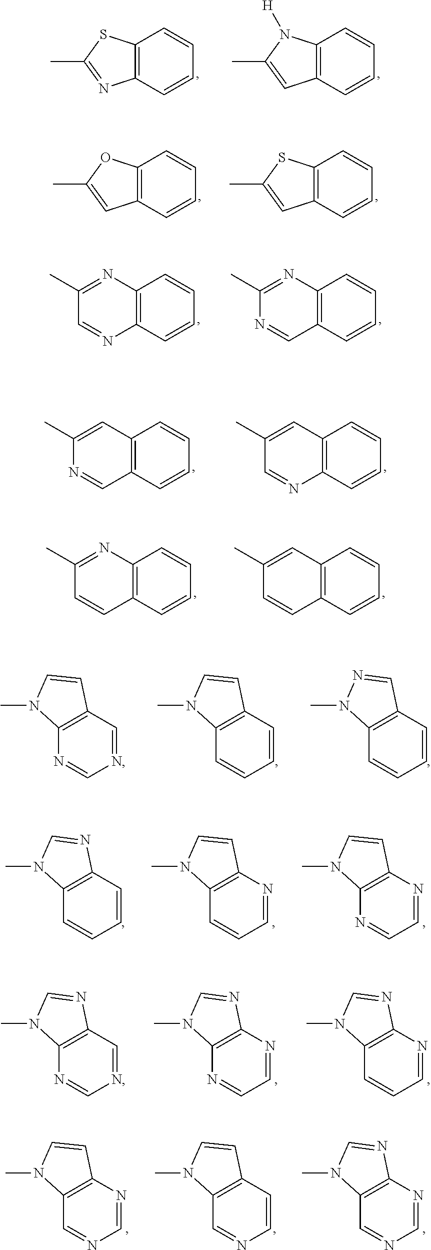 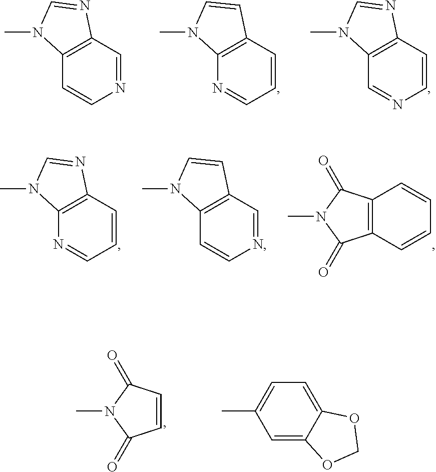
wherein
X is carbon or nitrogen and
R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph;
as well as salts, enantiomers, enantiomeric mixtures, diastereomers, diastereomeric mixtures, tautomers, hydrates, solvates and racemates of the aforementioned compounds.

12. The method according to claim 1, wherein the diaryl ureas have the following general structure: 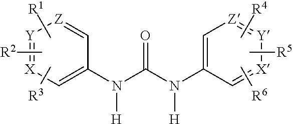
wherein
X, X′, Y, Y′, Z, Z′ are, independently of one another, carbon or nitrogen and R¹, R², R³, R⁴, R⁵, R⁶ are, independently of one another, C₁-C₆-alkyl, C₂-C₆-alkenyl, C₂-C₆-alkynyl, C₄-C₆-alkenynyl, C₃-C₁₀-Cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph as well as salts, enantiomers, enantiomeric mixtures, diastereomers, diastereomeric mixtures, tautomers, hydrates, solvates and racemates of the aforementioned compounds.

13. The method according to claim 1, wherein the 2H-indol-2-yliden-1-propene-1-ylindolium cations, benzothiazolyliden-1-propenyl-benzothiazolium cations and benzoxazolyiden-1-propenyl-benzoxazolium cations have the following general structure: 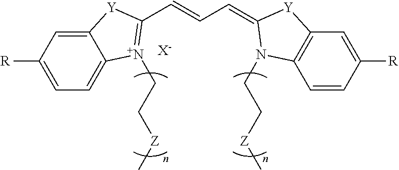
wherein
R represents hydrogen, —F, —Cl, —Br, —I, —NO₂, alkoxy;
X represents —Cl, —Br, —I, —OTs, —OMs;
Y represents O, S, CR¹R²;
wherein R¹ and R² independently of one another represent —CH₃ or —C₂H₅;
Z represents O or CH₂; and
n represents 0, 1, 2 or 3, as well as salts, enantiomers, enantiomeric mixtures, diastereomers, diastereomeric mixtures, tautomers, hydrates, solvates and racemates of the aforementioned compounds.

14. Arylaminothiazoles having the following general structure: 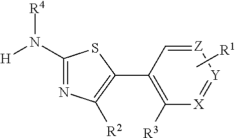
wherein
X, Y, Z are, independently of one another, carbon or nitrogen and
R¹, R², R³, R⁴ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-Alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph as well as salts, enantiomers, enantiomeric mixtures, diastereomers, diastereomeric mixtures, tautomers, hydrates, solvates and racemates of the aforementioned compounds.

15. 4,6-Divinylpyrimidines having the following general structure: 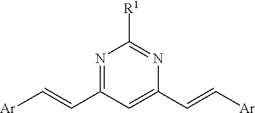
wherein
Ar represents one of the following cyclic, heterocyclic, aromatic or heteroaromatic groups: 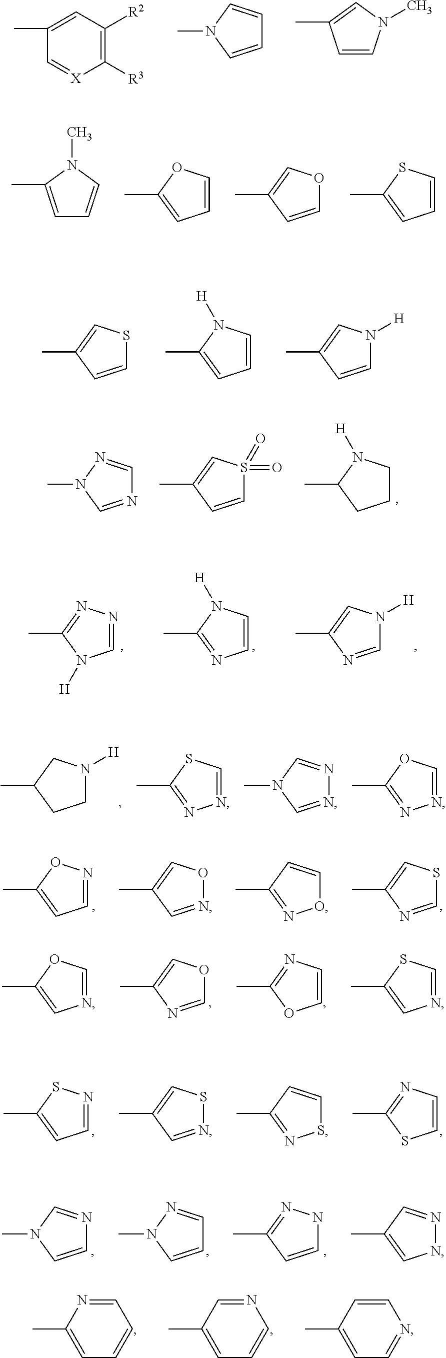 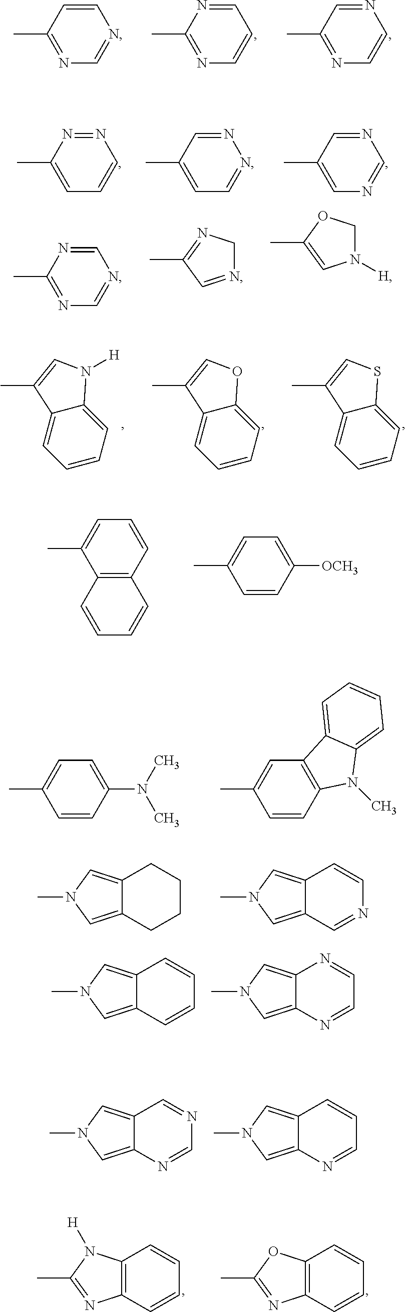 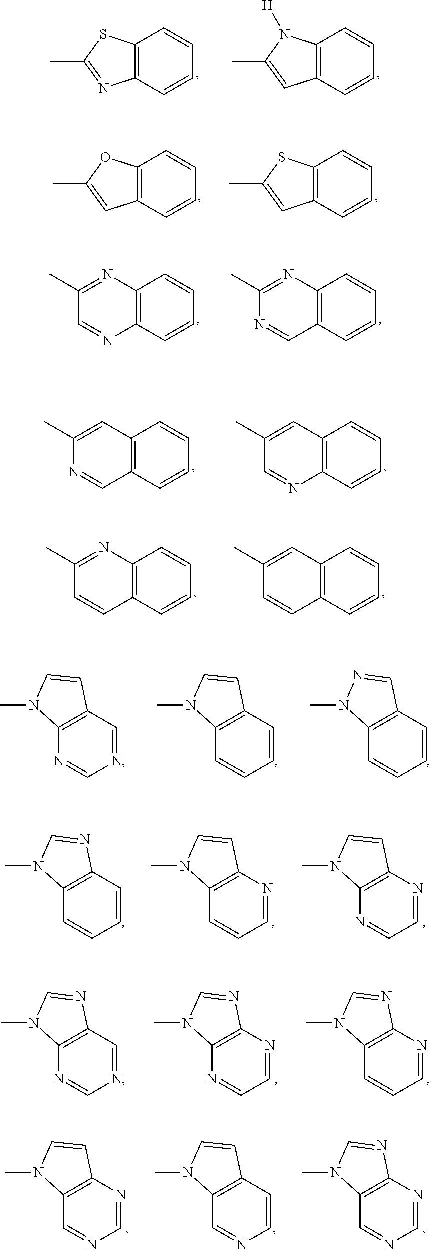 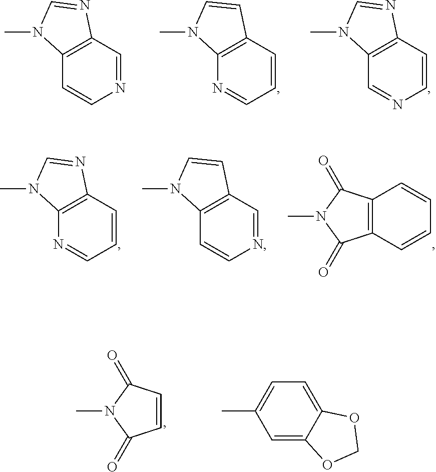
wherein
X is carbon or nitrogen and
R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph;
as well as salts, enantiomers, enantiomeric mixtures, diastereomers, diastereomeric mixtures, tautomers, hydrates, solvates and racemates of the aforementioned compounds.

16. 2,5-Divinylpyrazines having the following general structure: 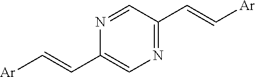
wherein
Ar represents one of the following cyclic, heterocyclic, aromatic or heteroaromatic groups: 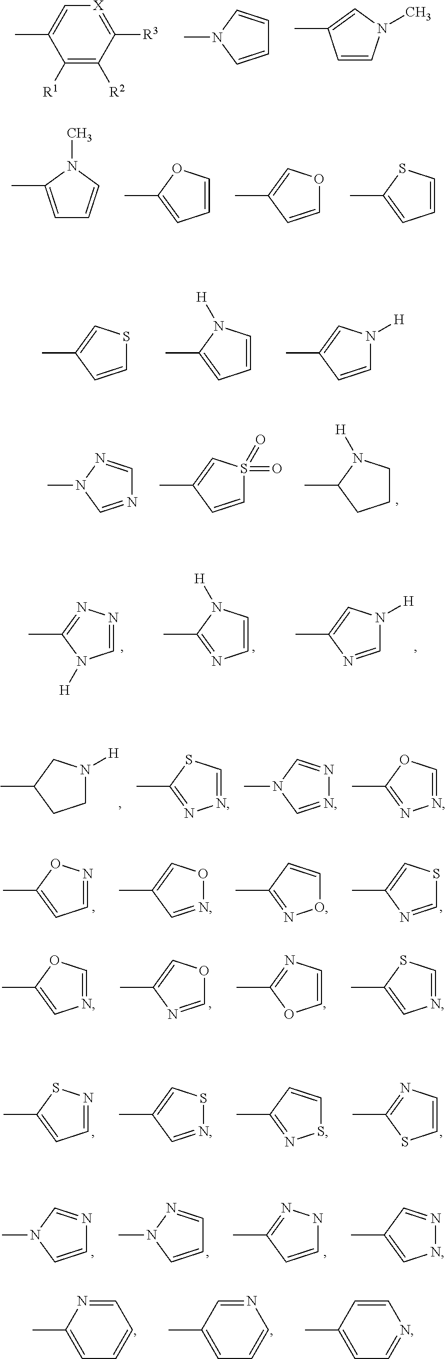 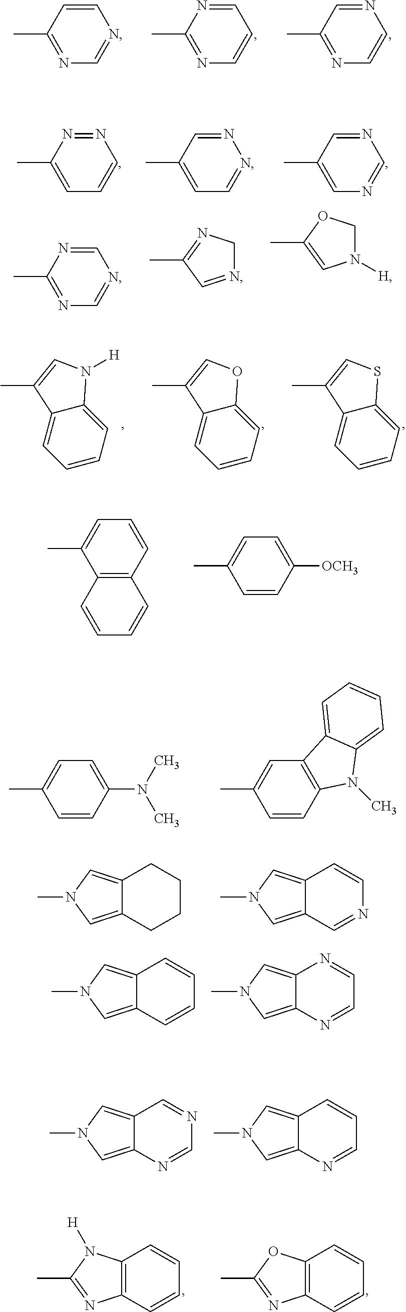 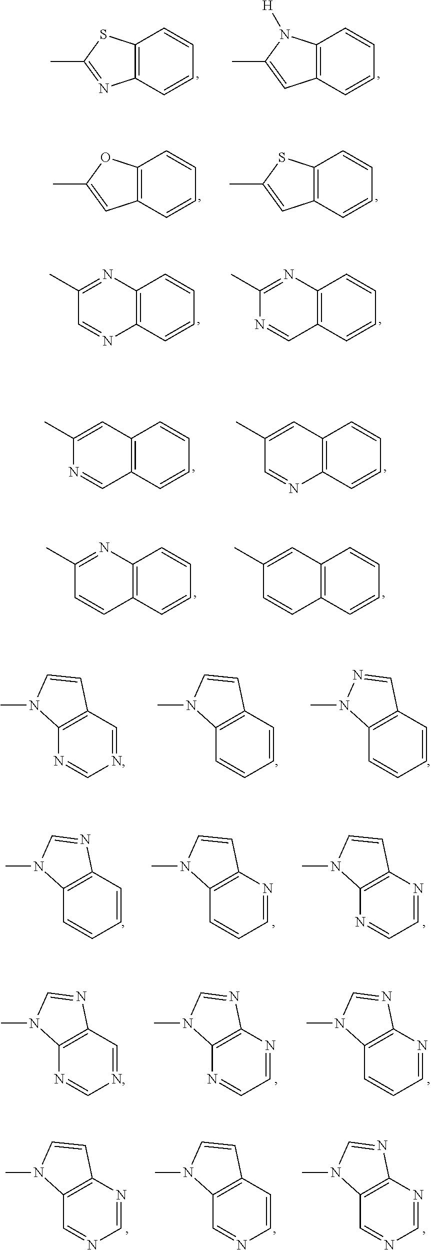 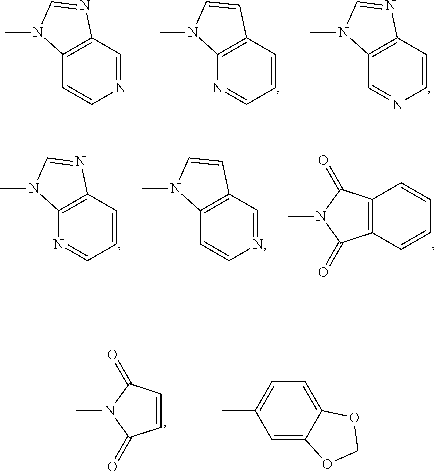
wherein
X is carbon or nitrogen and
R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph;
as well as salts, enantiomers, enantiomeric mixtures, diastereomers, diastereomeric mixtures, tautomers, hydrates, solvates and racemates of the aforementioned compounds.

17. [4-(1,3-Benzothiazol-2-yl)phenyl]hydrazones having the following general structure: 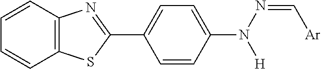
wherein
Ar represents one of the following cyclic, heterocyclic, aromatic or heteroaromatic groups: 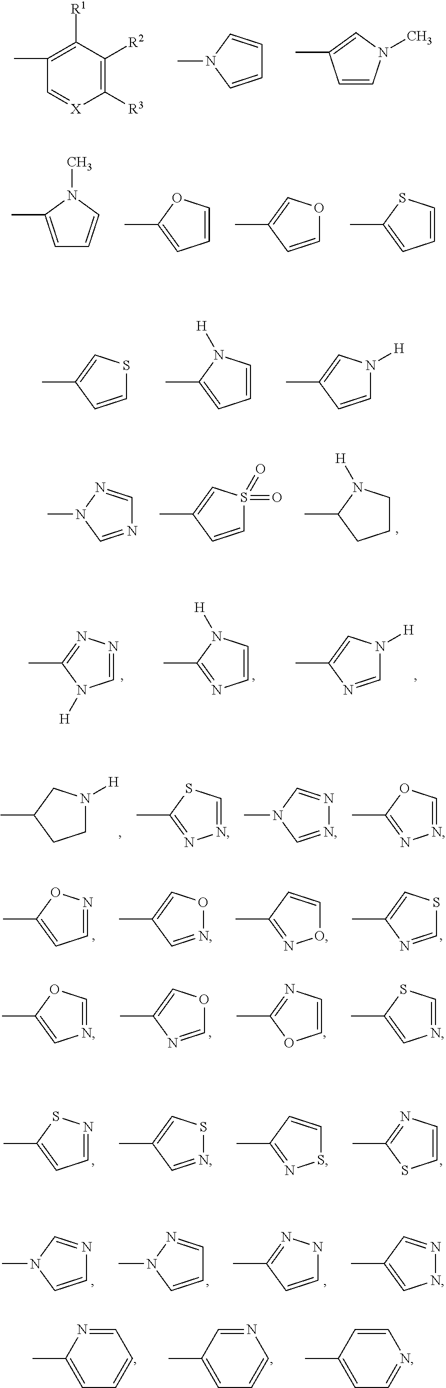 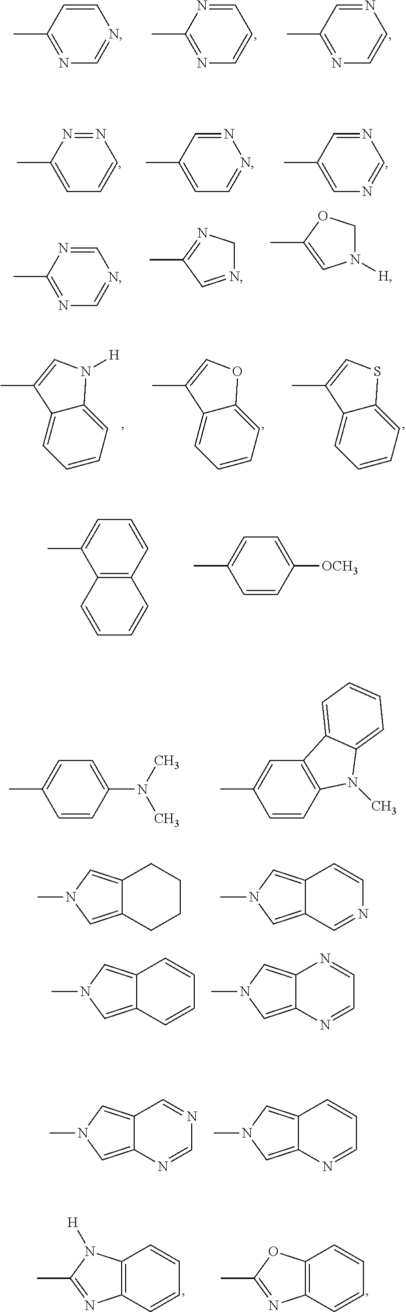 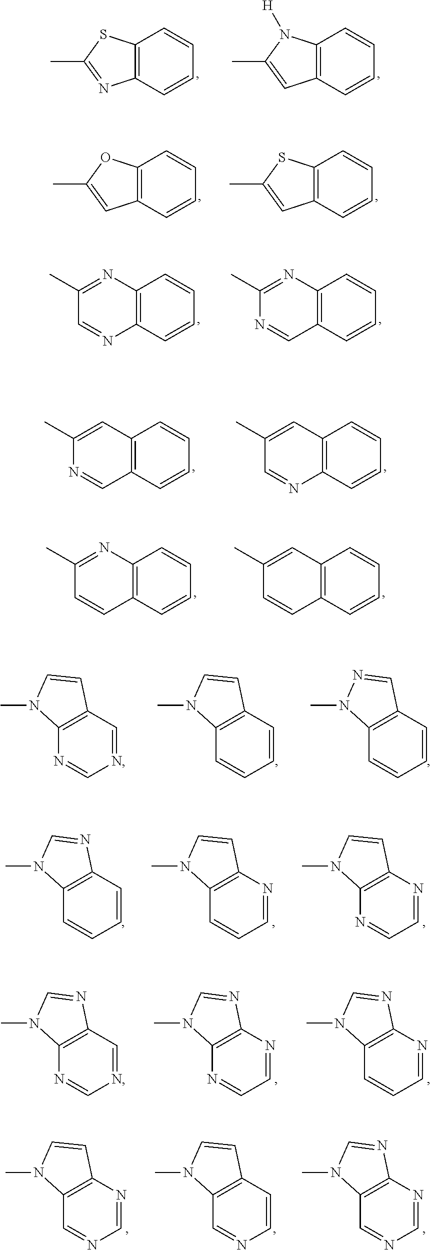 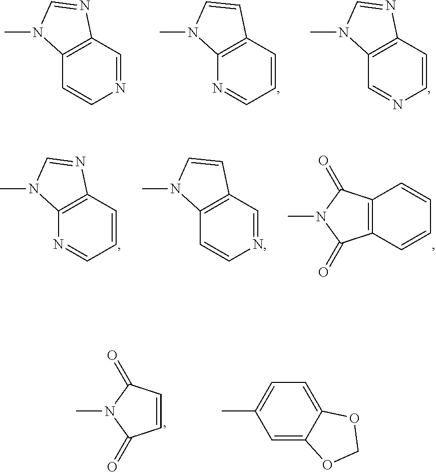
wherein
X is carbon or nitrogen and
R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-Alkyl)₂, -Ph, —CH₂-Ph, —CH═CH-Ph;
as well as salts, enantiomers, enantiomeric mixtures, diastereomers, diastereomeric mixtures, tautomers, hydrates, solvates and racemates of the aforementioned compounds.

18. 3,6-Divinylpyridazines having the following general structure: 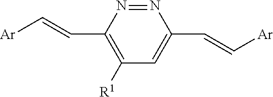
wherein
Ar represents one of the following cyclic, heterocyclic, aromatic or heteroaromatic groups: 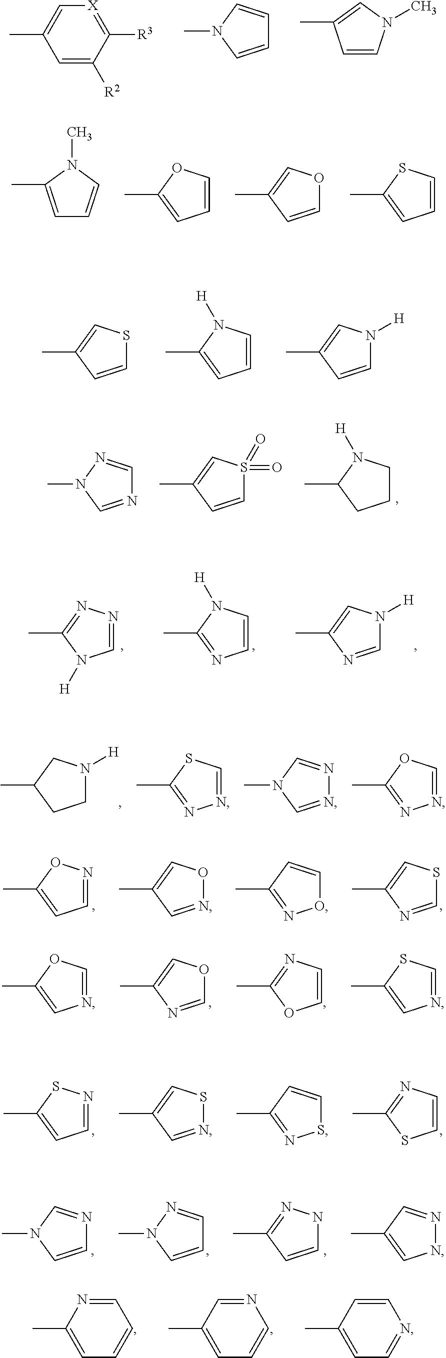 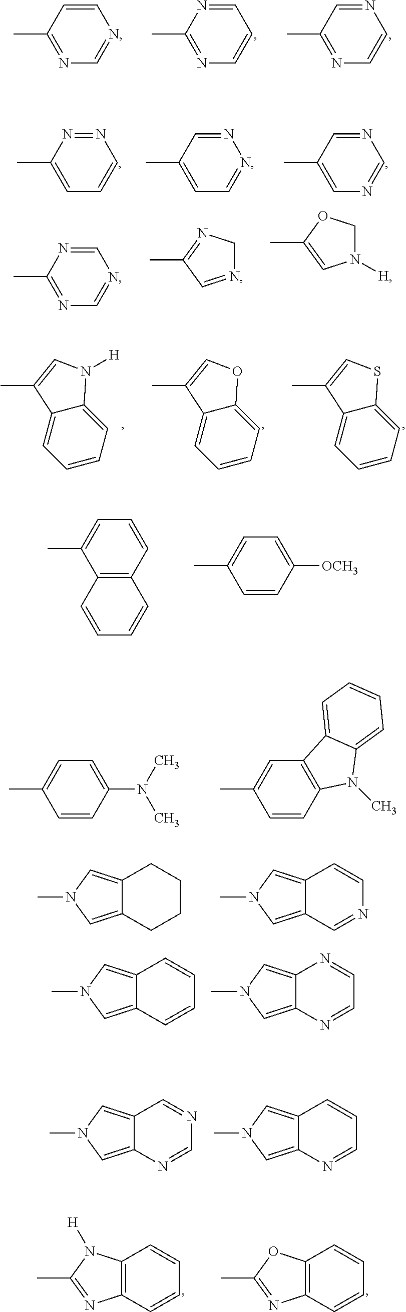 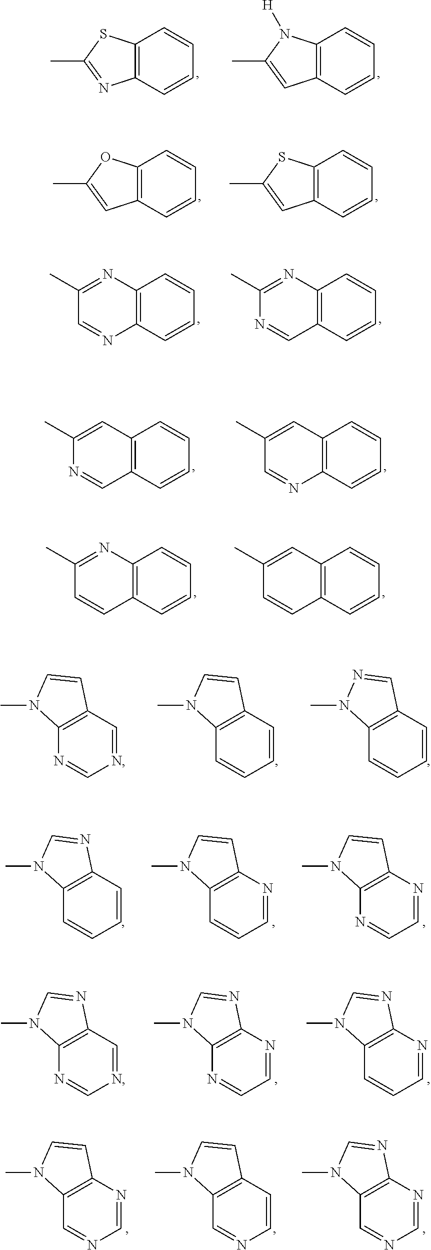 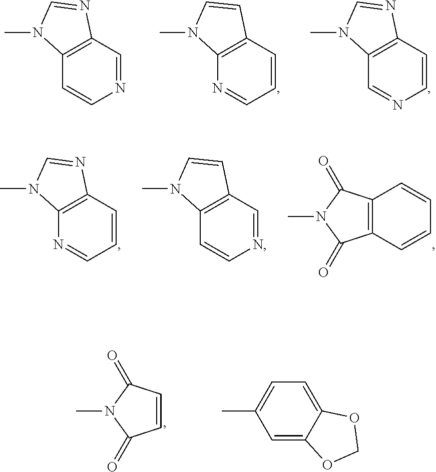
wherein
X is carbon or nitrogen and
R¹, R² and R³ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-Aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-Alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph;
as well as salts, enantiomers, enantiomeric mixtures, diastereomers, diastereomeric mixtures, tautomers, hydrates, solvates and racemates of the aforementioned compounds.

19. Diaryl ureas having the following general structure: 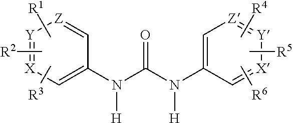
wherein
X, X′, Y, Y′, Z, Z′ are, independently of one another, carbon or nitrogen and R¹, R², R³, R⁴, R⁵, R⁶ are, independently of one another, C₁-C₆-alkyl, C₂-C₆-alkenyl, C₂-C₆-alkynyl, C₄-C₆-alkenynyl, C₃-C₁₀-cycloalkyl, thioalkyl, alkoxy, C₁-C₆-alkanoyl, C₆-C₁₆-aryl, C₆-C₁₆-heteroaryl, C₁-C₆-haloalkyl, C₂-C₆-haloalkenyl, C₂-C₆-haloalkynyl, C₄-C₆-haloalkenynyl, C₃-C₁₀-halocycloalkyl, —H, —OH, —OCH₃, —OC₂H₅, —OCF₃, —OC₂F₅, —NH₂, —N(CH₃)₂, —N(C₂H₅)₂, —SH, —SCH₃, —SC₂H₅, —COCH₃, —NO₂, —F, —Cl, —Br, —I, —P(O)(OH)₂, —P(O)(OCH₃)₂, —P(O)(OC₂H₅)₂, —COOH, —COO—C₁-C₆-alkyl, —COO—C₂-C₆-alkenyl, —COO—C₂-C₆-alkynyl, —O—CO—C₁-C₆-alkyl, —O—CO—C₂-C₆-alkenyl, —O—CO—C₂-C₆-alkynyl, —CONH₂, —NH—CO—C₁-C₆-alkyl, —NH—CO—C₂-C₆-alkenyl, —NH—CO—C₂-C₆-alkynyl, —CO—NH(C₁-C₆-alkyl), —CO—NH(C₂-C₆-alkenyl), —CO—NH(C₂-C₆-alkynyl), —CO—N(C₁-C₆-alkyl)₂, —CO—N(C₂-C₆-alkenyl)₂, —CO—N(C₂-C₆-alkynyl)₂, —NH(C₁-C₆-alkyl), —NH(C₂-C₆-alkenyl), —NH(C₂-C₆-alkynyl), —N(C₁-C₆-alkyl)₂, —N(C₂-C₆-alkenyl)₂, —N(C₂-C₆-alkynyl)₂, —SO—C₁-C₆-alkyl, —SO—C₂-C₆-alkenyl, —SO—C₂-C₆-alkynyl, —SO₂—C₁-C₆-alkyl, —SO₂—C₂-C₆-alkenyl, —SO₂—C₂-C₆-alkynyl, —SO₃H, —SO₃—C₁-C₆-alkyl, —SO₃—C₂-C₆-alkenyl, —SO₃—C₂-C₆-alkynyl, —SO₂NH₂, —O—COO—C₁-C₆-alkyl, —NH—CO—NH₂, —NH—CO—NH—C₁-C₆-alkyl, —NH—CO—N(C₁-C₆-alkyl)₂, -Ph, —CH₂-Ph, —CH═CH-Ph as well as salts, enantiomers, enantiomeric mixtures, diastereomers, diastereomeric mixtures, tautomers, hydrates, solvates and racemates of the aforementioned compounds.

20. 2H-Indol-2-yliden-1-propen-1-ylindolium cations, benzothiazolyliden-1-propenyl-benzothiazolium cations and benzoxazolyiden-1-propenyl-benzoxazolium cations having the following general structure: 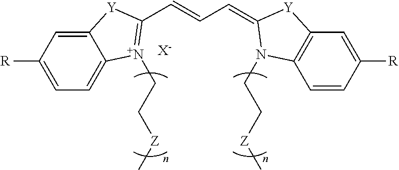
wherein
R represents hydrogen, —F, —Cl, —Br, —I, —NO₂, alkoxy;
X represents —Cl, —Br, —I, —OTs, —OMs;
Y represents O, S, CR¹R²;
wherein R¹ and R² independently of one another represent —CH₃ or —C₂H₅;
Z represents O or CH₂; and
n represents 0, 1, 2 or 3, as well as salts, enantiomers, enantiomeric mixtures, diastereomers, diastereomeric mixtures, tautomers, hydrates, solvates and racemates of the aforementioned compounds.

21. Ex-vivo method for the diagnosis of neurodegenerative disorders, comprising the following steps:
a) addition of a compound chosen from the group of arylaminothiazoles, 2H-indol-2-yliden-1-propen-1-ylindolium cations, benzothiazolyliden-1-propenyl-benzothiazolium cations, benzoxazolyiden-1-propenyl-benzoxazolium cations, 4,6-divinylpyrimidines, 3,6-divinylpyridazines, 2,5-divinylpyrazines, [4-(1,3-benzothiazol-2-yl)phenyl]hydrazones or diaryl ureas to a sample or biopsy of a patient with neurodegenerative disease, and
c) diagnosis of the neurodegenerative disease using fiber optics or fluorescence microscopy.
